(19) United States
(12) Patent Application Publication
(10) Pub. No.: US 2024/0383894 A1
(43) Pub. Date: Nov. 21, 2024

(54) HETEROBIDENTATE IMIDAZO[1,5-A]PYRIDINE AND IMIDAZO[1,5-A]QUINOLINE N-HETEROCYCLIC CARBENE (NHC) LIGANDS, CATALYST COMPLEXES THEREOF, AND METHODS USING SAME

(71) Applicant: Rutgers, The State University of New Jersey, New Brunswick, NJ (US)

(72) Inventors: Michal Szostak, Newark, NJ (US); Pengcheng Gao, Newark, NJ (US)

(21) Appl. No.: 18/722,028

(22) PCT Filed: Dec. 20, 2022
(86) PCT No.: PCT/US2022/053490

§ 371 Date: Jun. 20, 2024

Related U.S. Application Data
(60) Provisional application No. 63/292,235, filed on Dec. 21, 2021.

(51) Int. Cl. C07D 471/04; B01J 31/18; B01J 31/20; B01J 31/22; C07C 67/30; C07C 209/10; C07C 241/02; C07D 209/08; C07D 211/06; C07F 1/00; C07F 1/08; C07F 15/00

(52) U.S. Cl. CPC C07D 471/04; B01J 31/1805; B01J 31/20; B01J 31/2273; B01J 31/2291; B01J 31/2295; C07C 67/30; C07C 209/10; C07C 241/02; C07D 209/08; C07D 211/06; C07F 1/00; C07F 1/08; C07F 15/006; C07F 15/0073; B01J 2231/321; B01J 2231/4227; B01J 2231/4233; B01J 2231/4266; B01J 2231/4283; B01J 2531/004; B01J 2531/18; B01J 2531/60; B01J 2531/822; B01J 2531/824

(57) ABSTRACT

The present disclosure provides N-heterocyclic carbene ligands, catalyst complexes thereof, and methods using same. The present disclosure further provides synthetic methods of preparing the N-heterocyclic carbene ligands and catalyst complexes disclosed herein.

### CROSS-REFERENCE TO RELATED APPLICATIONS

The present application claims priority under 35 U.S.C. § 119(e) to U.S. Provisional Patent Application No. 63/292,235, filed Dec. 21, 2021, which is incorporated herein by reference in its entirety.

### STATEMENT REGARDING FEDERALLY SPONSORED RESEARCH

This invention was made with government support under grant number CHE-1650766 awarded by the National Science Foundation and grant number 1R35GM133326 awarded by the National Institutes of Health. The government has certain rights in this invention.

### BACKGROUND

Dalphos ligands, comprising a bulky di(1-adamantyl)phosphino [P(1-Adamantyl)₂] fragment, are among the most successful and widely used arylphosphine ligands in organic catalysis. These ligands, complexed with Au(I), Cu(I), Pd(0) or Ni(0) metals, have applications in Buchwald-Hartwig amination, ammonia arylation, hydrazine arylation, ketone arylation, C—O coupling, aminocarbonylation, carbonylative arylation, hydroamination, oxidative annulation, C—C coupling, C—S coupling, arylation of alkenes, Ni-catalyzed amide N-arylation, Ni-catalyzed sulfonamide N-arylation, Ni-catalyzed C—O coupling, Ni-catalyzed ammonia arylation, Ni-catalyzed amine arylation, Ni-catalyzed indole N-arylation, heteroarylamine arylations, Ni-catalyzed fluoroalkylamine arylations, Cu-catalyzed Suzuki coupling, Cu-catalyzed Hiyama coupling, and indium reagent couplings.

The unique properties of Dalphos ligands arise from a combination of soft and hard donor atoms, namely P and N, positioned in a 1,4-relationship (ortho-P,N-substitution), which leads to the formation of five-membered heterobidentate chelates with transition metals not easily available in other families of ligands. Presently, there are >20 DalPhos type ligands commercially available.

Despite the success of Dalphos ligands, the development of N-heterocyclic carbene (NHC) analogues has remained elusive due to the lack of suitable NHC scaffolds.

Thus, there is a need in the art for ligands that can support the heterobidentate chelation in a rigid, structurally-fixed NHC template. The present disclosure addresses this need.

### BRIEF SUMMARY OF THE INVENTION

In one aspect, the present disclosure provides a compound of formula (I), which is selected from the group consisting of:

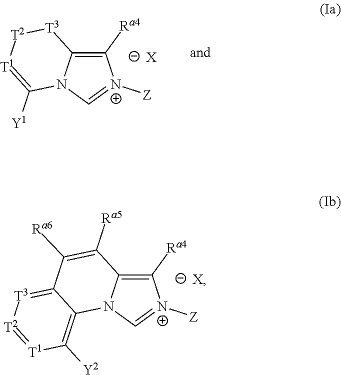

wherein:
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, R^{a3}, R^{a5}, and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
   wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen.

In another aspect, the present disclosure provides a method of preparing the compound of formula (Ia), the method comprising:
contacting a compound of formula (A):

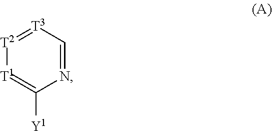

a compound of formula (B):

H₂N—Z (B), and
paraformaldehyde:

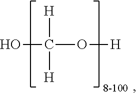

in the presence of an acid to form the compound of formula (Ia).

In another aspect, the present disclosure provides a compound of formula (II), which is selected from the group consisting of:

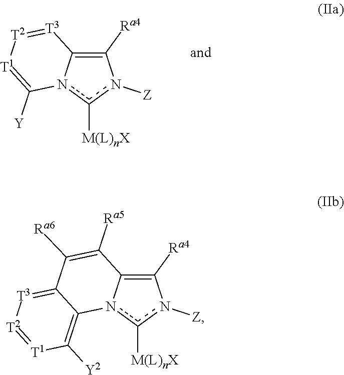

wherein:
M is a transition metal;
L is a ligand of M, wherein each occurrence of L can be the same or different;
each occurrence of [symbol] is a single or double bond,
   wherein no more than one [symbol] bonding a C atom and a N atom is a double bond;
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, R^{a3}, R^{a5} and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen; and
n is an integer which is selected from the group consisting of 0, 1, 2, and 3.

In another aspect, the present disclosure provides a method of promoting a reaction (coupling) between a first reagent and an aryl iodide (i.e., promoting arylation of a first reagent), the method comprising contacting the first reagent and the aryl iodide in the presence of the compound of formula (IIa) or (IIb), and optionally in the presence of a Lewis acid.

In another aspect, the present disclosure provides a method of promoting a reaction (coupling) between an aniline and an aryl iodide (i.e., promoting arylation of an aniline), the method comprising contacting the aniline and the aryl iodide in the presence of the compound of formula (IIa) or (IIb), and optionally in the presence of a Lewis acid.

In another aspect, the present disclosure provides a method of promoting a hydroamination reaction between an alkyne and an amine, the method comprising contacting the alkyne and the amine in the presence of the compound of formula (IIa) or (IIb), and optionally in the presence of a Lewis acid.

In another aspect, the present disclosure provides a method of promoting hydration of an alkyne, the method comprising contacting the alkyne and water in the presence of the compound of formula (IIa) or (IIb), and optionally in the presence of a Lewis acid.

In another aspect, the present disclosure provides a method of promoting a reaction (coupling) between an aryl chloride and a lithium amide (i.e., promoting arylation of an amide), the method comprising contacting the aryl chloride and the lithium amide in the presence of the compound of formula (IIa) or (IIb).

In another aspect, the present disclosure provides a method of promoting a reaction (coupling) between an aryl chloride and an arylmagnesium halide, the method comprising contacting the aryl chloride and the arylmagnesium halide in the presence of the compound of formula (IIa) or (IIb).

In another aspect, the present disclosure provides a method of promoting a reaction (coupling) between an aroyl chloride and an aryl boronic acid (i.e., promoting aroylation of an aryl reagent), the method comprising contacting the aroyl chloride and the aryl boronic acid in the presence of the compound of formula (IIa) or (IIb) and a base.

In another aspect, the present disclosure provides a method of promoting a reaction between an aryl iodide, an aniline, and carbon monoxide (CO), the method comprising contacting the aryl iodide, the aniline, and the CO in the presence of the compound of formula (IIa) or (IIb) and a base.

In another aspect, the present disclosure provides a method of preparing 2-(2,6-diisopropylphenyl)-5-(dimethylamino)imidazo[1,5-a]pyridin-2-ium chloride) (1):

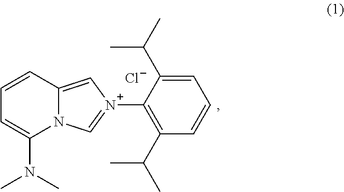 [SMILES: CC(C)c1cccc(C(C)C)c1-[n+]1cc2cccc(N(C)C)n2c1 ; [Cl-]]

the method comprising reacting (E)-6-(((2,6-diisopropylphenyl)imino)methyl)-N,N-dimethylpyridin-2-amine (A):

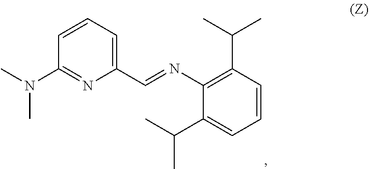 [SMILES: CC(C)c1cccc(C(C)C)c1/N=C/c1cccc(N(C)C)n1]

and paraformaldehyde so as to generate a first reaction system comprising (1).

### BRIEF DESCRIPTION OF THE FIGURES

The drawings illustrate generally, by way of example, but not by way of limitation, various embodiments of the present application.

FIG. 1: X-ray structure of 16 (front and side view).

FIG. 2: X-ray structure of 22 (front and side view).

FIG. 3: X-ray structure of 18 (front and side view).

FIG. 4: Oxidative Au(I)/Au(III) C—C coupling by C—H arylation: substrate scope; ^{a}Conditions: ArH (1.0 equiv), ArI (1.0 equiv), AgNTf₂ (1.1 equiv), [Au] 5 mol %, MeOH (0.125 M), 80° C., 16 h; ^{b}Conditions: ArH (2.0 equiv), ArI (1.0 equiv), AgNTf₂ (1.1 equiv), [Au] 5 mol %, MeOH (0.125 M), 80° C., 16 h.

FIG. 5: Oxidative Au(I)/Au(III) C—C coupling by C—H heteroarylation: substrate scope; ^{a}Conditions HetArH (2.0 equiv), ArI (1.0 equiv), AgNTf₂ (1.1 equiv), [Au] 5 mol %, MeOH (0.125 M), 80° C., 16 h.

FIG. 6: Oxidative Au(I)/Au(III) C—N coupling by C—X amination: substrate scope; ^{a}Conditions: ArNH₂ (1.0 equiv), ArI (1.0 equiv), AgNTf₂ (1.1 equiv), [Au] 2.5 mol %, MeOH (0.125 M), 80° C., 16 h; ^{b}Conditions: ArNH₂ (1.0 equiv) or R—NH₂ (2.0 or 3.0 equiv), ArI (1.0 equiv), AgNTf₂ (1.1 equiv), [Au] 5 mol %; MeOH (0.125 M), 80° C., 16 h.

FIG. 7: Electrophilic Au(I) hydroamination of internal alkynes with aliphatic amines: substrate scope; ^{a}Conditions: (1) Alkyne (1.0 equiv), amine (1.2 equiv), [Au] 2.5 mol %, NaBAr^{F}₄ 5 mol %, toluene (0.5 M), 110° C., 16 h; (2) (CH₃COO)₃BHNa (2.0 equiv), AcOH (2.0 equiv), CH₂Cl₂ (0.2 M), RT, 24 h.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### INTRODUCTION

In one aspect, the ligands disclosed herein exploit the unique coordination of soft and hard donor atoms in a 1,4-relationship to form five-membered heterobidentate chelates. In further embodiments, the ligand disclosed herein exploit the unique coordination of soft and hard donor atoms in a 1,5-relationship to form six-membered heterobidentate chelates. In certain embodiments, catalysts of the present disclosure demonstrate utility in oxidative gold(I)/(III) catalysis, an important area of contemporary transition-metal catalysis. Despite the significance of such catalysis, N-heterocyclic carbene (NHC) ligands capable of promoting such catalysis have not been developed to date, and all examples are limited to phosphines.

Specifically, imidazo[1,5-a]pyridine architecture results in the following features relevant to catalysis: (1) amino-decorated L-shaped ligands have similar geometry of the active site of DalPhos ligands, wherein the carbene center and the nitrogen atom are connected through 3 covalent bonds in a fixed conformation (C—N, C—N and C—N bonds) cf. (P—C, C—C, C—N bonds) in DalPhos; (2) the connecting atoms are coplanar; (3) NHC ligands are significantly more σ-donating and more π-accepting than phosphine ligands, thus facilitating difficult oxidative additions and providing additional stabilization for transition metals in high oxidation states; (4) the steric bulk of amino-decorated L-shaped ligands is unique due to L-shape wall-restricted umbrella-type steric distribution, resulting in a tighter coordination angle between the soft and hard donor atoms and transition metals. Overall, amino-decorated L-shaped ligands have unique properties compared with DalPhos ligands and all other NHC ligands developed to date.

Furthermore, Au(I)/(Au(III) oxidative gold catalysis is unprecedented using NHC ligands. Other NHC ligands do not catalyze Au(I)/Au(III) reactions. The present class of ligands is the first class of NHCs that can be utilized in oxidative gold catalysis.

Without wishing to be bound by theory, it has been proposed that the high reactivity in Au(I)/(III) catalysis is due to heterobidentate stabilization of Au(I) and Au(III) intermediates through coordination of soft carbene and hard nitrogen donor atoms in a 1,4-relationship.

Furthermore, the present ligands show higher reactivity in electrophilic Au(I) amination than the standard NHC ligands developed to date.

It has been proposed that the high reactivity in electrophilic Au(I) catalysis results from lower lying LUMO of amino-decorated L-shaped ligands in combination with a directing role of amine through hydrogen bonding. Summaries of catalytic studies, along with the effect of ligands, are described herein.

### Definitions

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aniline” as used herein refers to an amine having at least one aryl or heteroaryl substituent.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aroyl” as used herein refers to an aryl group, as defined elsewhere herein, substituted at any one position with a carbonyl moiety (i.e., C(═O)). Use of the term “aroyl” in combination with another term (e.g., chloride), indicates that the carbonyl linked to the aryl group is substituted with the substituent defined by the term used in the combination. For example, PhC(═O)Cl (i.e., benzoyl chloride) is an aroyl chloride.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof. Use of the term “aryl” in combination with another term (e.g., iodide, chloride, boronic acid, and magnesium halide, inter alia), indicates that the aryl group is substituted at one or more positions with the substituent defined by the term used in the combination. For example, an aryl chloride indicates that the aryl is substituted with at least one chloride.

The term “atm” as used herein refers to a pressure in atmospheres under standard conditions. Thus, 1 atm is a pressure of 101 kPa, 2 atm is a pressure of 202 kPa, and so on.

The term “counter anion” as used herein refers to a negatively charged ion that accompanies a cationic species (i.e. positively charged ion) in order to maintain electric neutrality. For example, the chloride ion (Cl⁻) is the counter anion to sodium (Na⁺) in NaCl. Non-limiting examples of counter anions include F⁻, Cl⁻, Br⁻, I⁻, H⁻, N₃⁻, PhCH═CHCH₂⁻, PhCH⁻ CH═CH₂, F₃CS(═O)₂O⁻ (OTf), (F₃CS(═O)₂)₂N⁻ (NTf₂), F₃CC(═O)O⁻ (TFA), BF₄⁻, and PF₆⁻.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “electrophile” as used herein refers to a chemical species that forms a bond with a nucleophile by accepting an electron pair in a chemical reaction (e.g. S_{N}1, S_{N}2, and carbonyl [1,2]-addition). Non-limiting examples of electrophiles include alkyl halides (e.g. MeI), benzyl halides (e.g. BnBr), dihalides (e.g. Br₂), aldehydes (e.g. Ph-CHO), acyl halides (e.g. Ph(C═O)Cl), N-electrophiles (e.g. RC(═O)ONR₂), and O-electrophiles (e.g. RC(═O)O₂R).

The terms “epoxy-functional” or “epoxy-substituted” as used herein refers to a functional group in which an oxygen atom, the epoxy substituent, is directly attached to two adjacent carbon atoms of a carbon chain or ring system. Examples of epoxy-substituted functional groups include, but are not limited to, 2,3-epoxypropyl, 3,4-epoxybutyl, 4,5-epoxypentyl, 2,3-epoxypropoxy, epoxypropoxypropyl, 2-glycidoxyethyl, 3-glycidoxypropyl, 4-glycidoxybutyl, 2-(glycidoxycarbonyl)propyl, 3-(3,4-epoxycylohexyl)propyl, 2-(3,4-epoxycyclohexyl)ethyl, 2-(2,3-epoxycylopentyl)ethyl, 2-(4-methyl-3,4-epoxycyclohexyl)propyl, 2-(3,4-epoxy-3-methylcylohexyl)-2-methylethyl, and 5,6-epoxyhexyl.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaralkynyl” as used herein refers to alkynyl groups as defined herein in which a hydrogen or carbon bond of an alkynyl group is replaced with a bond to a heteroaryl group as defined herein. Representative aralkynyl groups include, but are not limited to, 2-ethynylpyridine and 2-ethynylthiophene.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “IMes” as used herein refers to 1,3-bis(2,4,6-trimethylphenyl)-1,3-dihydro-2H-imidazol-2-ylidene.

The term “IPr” as used herein refers to 1,3-bis(2,6-diisopropylphenyl)-1,3-dihydro-2H-imidazol-2-ylidene.

The term “IPr*” as used herein refers to 1,3-bis((2,6-(dibenzhydryl))-4-methylphenyl)-1,3-dihydro-2H-imidazol-2-ylidene.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “Lewis acid” as used herein refers to a chemical species that possesses an empty orbital which is capable of accepting a pair of non-bonding electrons from a species having a filled orbital containing an electron pair (i.e., a Lewis base).

The term “lithium amide” as used herein refers to a lithium salt of an anionic amine of the formula N(R)(R), wherein R is selected from the group consisting of H, C₁-C₁₂ alkyl, and Si(C₁-C₁₂ alkyl)₃. Non-limiting examples of lithium amides include lithium diisopropylamide (LDA) and lithium hexadimethylsilazide (LiHMDS).

The terms “[M(#)X]” or “[M(NHC)X]” as used herein indicate a N-heterocyclic carbene (NHC) complex comprising a metal (M) (e.g., Au, Pd, Ag, Cu, and Rh), an anionic counter ion (X) (e.g., Cl), and an NHC derived from an imidazolium salt (i.e., NHC or compound #). Additional terms may be included to indicate further ligands and/or counter ions. In such compounds, the compound number or name provided for the NHC may correspond to an imidazolium salt, however, the catalyst complex comprises the carbene derived from the indicated imidazolium salt complexed to the indicated metal.

The term “monovalent” as used herein refers to a substituent connecting via a single bond to a substituted molecule. When a substituent is monovalent, such as, for example, F or Cl, it is bonded to the atom it is substituting by a single bond.

The term “neutral ligand” or “ligand” as used herein, refers to a ligand having no net charge prior to association with, or after dissociation from, a metal center. Non-limiting examples of neutral ligands include alkene (e.g., cyclooctadiene), CO, amine (e.g., NMe₃), phosphine (e.g., PPh₃), and pyridyl ligands, wherein coordination occurs via the nitrogen lone pair of the pyridyl group.

The term “nitrile” as used herein refers to an organic compound comprising a cyano group (C≡N).

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)², N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “protic solvent” as used herein refers to a solvent that has a hydrogen atom bound to a heteroatom such as O, N, or S, such that the H⁺ is labile. Non-limiting examples of protic solvents include methanol, ethanol, isopropanol, acetic acid, water, n-butanol, and formic acid. Conversely, an “aprotic solvent” as used herein refers to a solvent lacking dissociable hydrogen ions (i.e. non-acidic) to an appreciable extent. Non-limiting examples of aprotic solvents include ethyl acetate (EtOAc), diethyl ether (Et₂O), tetrahydrofuran (THF), dimethylformamide (DMF), and 1,4-dioxane.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O(oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

### Preparation of Compounds

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

The compounds of the present disclosure can be prepared by the general schemes described herein, using the synthetic method known by those skilled in the art. The following examples illustrate non-limiting embodiments of the compound(s) described herein and their preparation.

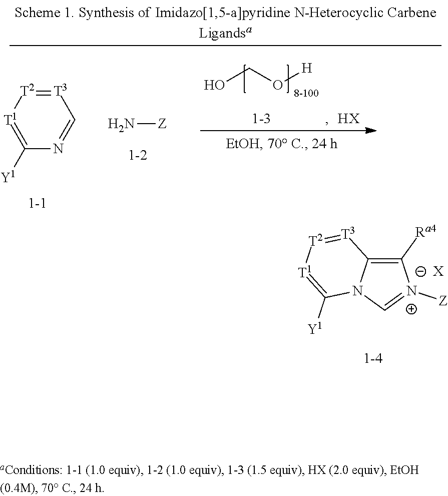

The N-heterocyclic carbene ligands of the present disclosure were prepared according to Scheme 1, wherein T¹, T², T³, R^{a1}, R^{a2}, R^{a3}, X, Y, and Z are defined within the scope of the present disclosure.

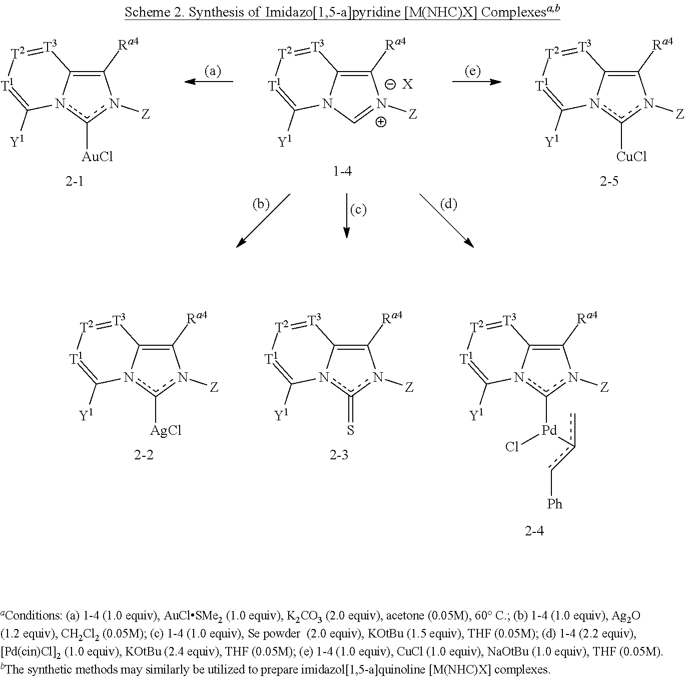

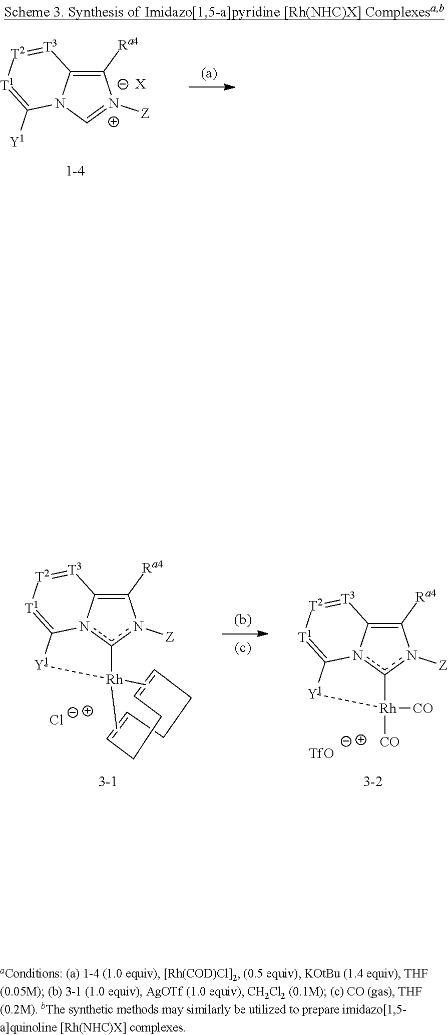

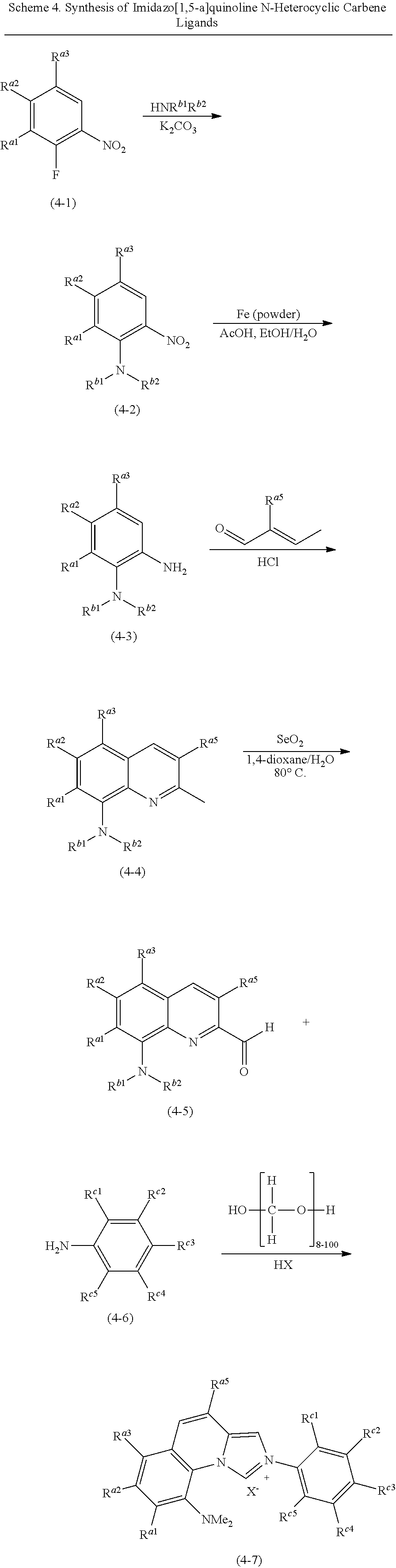

The N-heterocyclic carbene (NHC) complexes of the present disclosure were prepared as described herein, non-limiting embodiments of which are provided in Schemes 1-4, wherein T¹, T², T³, R^{a1}, R^{a2}, R^{a3}, R^{a4}, R^{a5}, R^{b1}, R^{b2}, R^{c1}, R^{c2}, R^{c3}, R^{c4}, R^{c5}, X, Y¹, Y², and Z are defined within the scope of the present disclosure.

### Compounds

The present disclosure relates in part to a compound of formula (I), which is selected from the group consisting of:

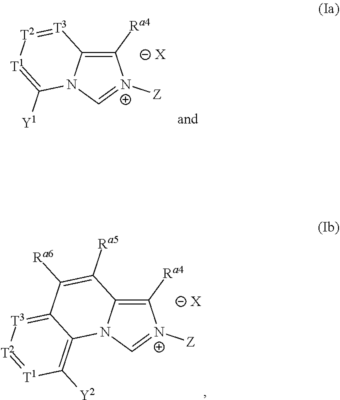

wherein:
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B} N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, and R^{a3}, is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl; and
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
   wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen.

In certain embodiments, none of T¹, T², and T³ is N. In certain embodiments, one of T¹, T², and T³ is N. In certain embodiments, two of T¹, T², and T³ are N. In certain embodiments, three of T¹, T², and T³ are N.

In certain embodiments, T¹ is CR^{a1}, T² is CR^{a2}, and T³ is CR^{a3}. In certain embodiments, T¹ is N, T² is CR^{a2}, and T³ is CR^{a3}. In certain embodiments, T¹ is CR^{a1}, T² is N, and T³ is CR^{a3} In certain embodiments, T¹ is CR^{a1}, T² is CR^{a2}, and T³ is N. In certain embodiments, T¹ is N, T² is N, and T³ is CR^{a3}. In certain embodiments, T¹ is N, T² is CR^{a2}, and T³ is N. In certain embodiments, T¹ is CR^{a1}, T² is N, and T³ is N. In certain embodiments, T¹ is N, T² is N, and T³ is N. In certain embodiments, T¹ is CH, T² is CH, and T³ is CH.

In certain embodiments, Z is:

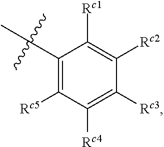

wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{c1}, R^{c2}, R^{c3}, R^{c4}, and R^{c5} may combine with the carbon atoms to which they are bound to form an optionally substituted C₅-C₁₂ cycloalkyl, optionally substituted C₂-C₁₂ heterocyclyl, or optionally substituted C₆-C₁₀ aryl.

In certain embodiments, one of R^{a1}, R^{a2}, and R^{a3} is H. In certain embodiments, two of R^{a1}, R^{a2}, and R^{a3} are H. In certain embodiments, each of R^{a1}, R^{a2}, and R^{a3} are H.

In certain embodiments, X is selected from the group consisting of OS(═O)₂R^{A}, OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl

In certain embodiments, X is Cl.

In certain embodiments, R^{b1} and R^{b2} can combine with the nitrogen atom to which they are bound to form a morpholinyl. In certain embodiments, R^{b1} and R^{b2} can combine with the nitrogen atom to which they are bound to form a piperidinyl. In certain embodiments, R^{b1} and R^{b2} can combine with the nitrogen atom to which they are bound to form a fluorenyl. In certain embodiments, R^{b1} and R^{b2} can combine with the nitrogen atom to which they are bound to form an optionally substituted pyrazolyl.

In certain embodiments, R^{b1} is H. In certain embodiments R^{b1} is methyl. In certain embodiments, R^{b1} is ethyl. In certain embodiments, R^{b1} is n-propyl. In certain embodiments, R^{b1} is i-propyl. In certain embodiments, R^{b1} is n-butyl. In certain embodiments, R^{b1} is sec-butyl. In certain embodiments, R^{b1} is i-butyl.

In certain embodiments, R^{b2} is H. In certain embodiments R^{b2} is methyl. In certain embodiments, R^{b2} is ethyl. In certain embodiments, R^{b2} is n-propyl. In certain embodiments, R^{b2} is i-propyl. In certain embodiments, R^{b2} is n-butyl. In certain embodiments, R^{b2} is sec-butyl. In certain embodiments, R^{b2} is i-butyl.

In certain embodiments, Y¹ is OMe. In certain embodiments, Y is NMe₂. In certain embodiments, Y¹ is NEt₂. In certain embodiments, Y¹ is

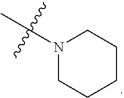 [SMILES: *N1CCCCC1]

In certain embodiments, Y¹ is

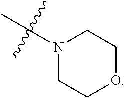 [SMILES: *N1CCOCC1]

In certain embodiments, Y¹ is

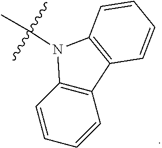 [SMILES: *n1c2ccccc2c2ccccc21]

In certain embodiments, Y¹ is

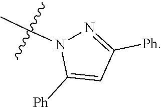 [SMILES: *n1nc(-c2ccccc2)cc1-c1ccccc1]

In certain embodiments, Y² is NMe₂. In certain embodiments, Y² is NEt₂. In certain embodiments, Y² is

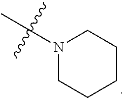 [SMILES: *N1CCCCC1]

In certain embodiments, Y² is

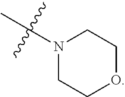 [SMILES: *N1CCOCC1]

In certain embodiments, Y² is

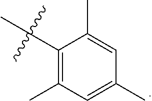

In certain embodiments, R^{c1} is methyl. In certain embodiments, R^{c1} is i-propyl. In certain embodiments, R^{c1} is diphenylmethyl.

In certain embodiments, R^{c5} is methyl. In certain embodiments, R^{c5} is i-propyl. In certain embodiments, R^{c5} is diphenylmethyl.

In certain embodiments, R^{c1} and R^{c5} are identical. In certain embodiments, R^{c1} is methyl and R^{c5} is methyl. In certain embodiments, R^{c1} is i-propyl and R^{c5} is i-propyl. In certain embodiments, R^{c1} is diphenylmethyl and R^{c5} is diphenylmethyl.

In certain embodiments, R^{c3} is H. In certain embodiments, R^{c3} is methyl.

In certain embodiments, R^{c2} is H. In certain embodiments, R^{c4} is H. In certain embodiments, R^{c2} and R^{c4} are H.

In certain embodiments, Z is

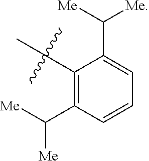 [SMILES: *c1c(C(C)C)cccc1C(C)C]

In certain embodiments, Z is

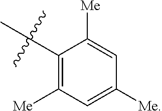 [SMILES: *c1c(C)cc(C)cc1C]

In certain embodiments, Z is

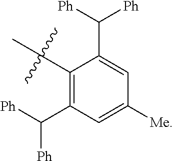 [SMILES: *c1c(C(c2ccccc2)c2ccccc2)cc(C)cc1C(c1ccccc1)c1ccccc1]

In certain embodiments, the compound of formula (Ia) or (Ib) is selected from the group consisting of:
2-(2,6-diisopropylphenyl)-5-(dimethylamino)imidazo[1,5-a]pyridin-2-ium chloride;
5-(dimethylamino)-2-mesitylimidazo[1,5-a]pyridin-2-ium chloride;
2-(2,6-dibenzhydryl-4-methylphenyl)-5-(dimethylamino)imidazo[1,5-a]pyridin-2-ium chloride;
5-(diethylamino)-2-(2,6-diisopropylphenyl)imidazo[1,5-a]pyridin-2-ium chloride;
5-(diethylamino)-2-mesitylimidazo[1,5-a]pyridin-2-ium chloride;
2-(2,6-dibenzhydryl-4-methylphenyl)-5-(diethylamino)imidazo[1,5-a]pyridin-2-ium chloride;
2-(2,6-diisopropylphenyl)-5-(piperidin-1-yl)imidazo[1,5-a]pyridin-2-ium chloride;
2-mesityl-5-(piperidin-1-yl)imidazo[1,5-a]pyridin-2-ium chloride;
2-(2,6-dibenzhydryl-4-methylphenyl)-5-(piperidin-1-yl)imidazo[1,5-a]pyridin-2-ium chloride;
2-(2,6-diisopropylphenyl)-5-morpholinoimidazo[1,5-a]pyridin-2-ium chloride;
2-mesityl-5-morpholinoimidazo[1,5-a]pyridin-2-ium chloride;
2-(2,6-dibenzhydryl-4-methylphenyl)-5-morpholinoimidazo[1,5-a]pyridin-2-ium chloride;
2-(2,6-diisopropylphenyl)-5-(3,5-diphenyl-1H-pyrazol-1-yl)imidazo[1,5-a]pyridin-2-ium chloride;
5-(9H-carbazol-9-yl)-2-(2,6-diisopropylphenyl)imidazo[1,5-a]pyridin-2-ium chloride;
2-(2,6-diisopropylphenyl)-5-methoxyimidazo[1,5-a]pyridin-2-ium chloride;
2-(2,6-Diisopropylphenyl)-9-(dimethylamino)imidazo[1,5-a]quinolin-2-ium chloride;
2-(2,6-Diisopropylphenyl)-9-(diethylamino)imidazo[1,5-a]quinolin-2-ium chloride;
2-(2,6-Diisopropylphenyl)-9-(piperidin-1-yl)imidazo[1,5-a]quinolin-2-ium chloride;
2-(2,6-Diisopropylphenyl)-9-morpholinoimidazo[1,5-a]quinolin-2-ium chloride; 9-(Dimethylamino)-2-mesitylimidazo[1,5-a]quinolin-2-ium chloride; and
2-(2,6-Diisopropylphenyl)-9-(2,4,6-trimethylphenyl)imidazo[1,5-a]quinolin-2-ium chloride.

In certain embodiments, the compound of formula (I) is a compound of formula (II), which is selected from the group consisting of:

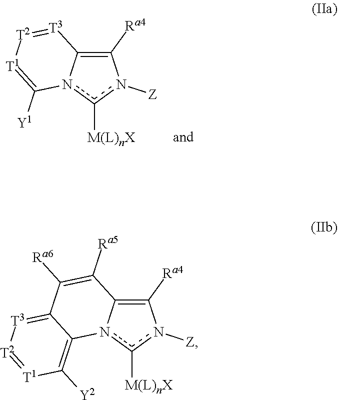

wherein:
M is a transition metal;
L is a ligand of M, wherein each occurrence of L can be the same or different;
each occurrence of [symbol] is a single or double bond,
   wherein no more than one [symbol] bonding a C atom and a N atom is a double bond;
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₅ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, and R^{a3}, is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl; and
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
   wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen; and
n is an integer which is selected from the group consisting of 0, 1, 2, and 3.

In certain embodiments, none of T¹, T², and T³ is N. In certain embodiments, one of T¹, T², and T³ is N. In certain embodiments, two of T¹, T², and T³ are N. In certain embodiments, three of T¹, T², and T³ are N.

In certain embodiments, T¹ is CR^{a1}, T² is CR^{a2}, and T³ is CR^{a3}. In certain embodiments, T¹ is N, T² is CR^{a2}, and T³ is CR^{a3}. In certain embodiments, T¹ is CR^{a1}, T² is N, and T³ is CR^{a3} In certain embodiments, T¹ is CR^{a1}, T² is CR^{a2}, and T³ is N. In certain embodiments, T¹ is N, T² is N, and T³ is CR^{a3}. In certain embodiments, T¹ is N, T² is CR^{a2}, and T³ is N. In certain embodiments, T¹ is CR^{a1}, T² is N, and T³ is N. In certain embodiments, T¹ is N, T² is N, and T³ is N. In certain embodiments, T¹ is CH, T² is CH, and T³ is CH.

In certain embodiments, Z is:

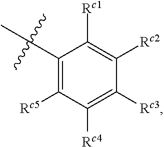

wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{c1}, R^{c2}, R^{c3}, R^{c4}, and R^{c5} may combine with the carbon atoms to which they are bound to
form an optionally substituted C₅-C₁₂ cycloalkyl, optionally substituted C₂-C₁₂
heterocyclyl, or optionally substituted C₆-C₁₀ aryl.

In certain embodiments, M is Au. In certain embodiments, M is Cu. In certain embodiments, M is Ag. In certain embodiments, M is Pd. In certain embodiments, M is Ni. In certain embodiments, M is Pt. In certain embodiments, M is Co. In certain embodiments, M is Rh. In certain embodiments, M is Ir. In certain embodiments, M is Fe. In certain embodiments, M is Ru. In certain embodiments, M is Os.

In certain embodiments, X is selected from the group consisting of H, OS(═O)₂R^{A} OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

In certain embodiments, X is Cl. In certain embodiments, X is trifluoromethanesulfonate (OTf). In certain embodiments, X is bis(trifluoromethansulfonyl)amide (NTf₂). In certain embodiments, X is allylbenzene anion (i.e., 3-phenylpropen-3-ide and/or 1-phenylpropen-3-ide).

In certain embodiments, wherein L is selected from the group consisting of Y, carbon monoxide (CO), optionally substituted C₂-C₁₂ alkene, and optionally substituted C₅-C₁₂ cycloalkene, wherein each optional substituent in the C₂-C₁₂ alkene and C₅-C₁₂ cycloalkene is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

In certain embodiments, wherein L is cyclooctadiene (COD). In certain embodiments, L is carbon monoxide (CO).

In certain embodiments, one of R^{a1}, R^{a2}, and R^{a3} is H. In certain embodiments, two of R^{a1}, R^{a2}, and R^{a3} are H. In certain embodiments, each of R^{a1}, R^{a2}, and R^{a3} are H.

In certain embodiments, X is selected from the group consisting of OS(═O)₂R^{A}, OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl

In certain embodiments, X is Cl.

In certain embodiments, R^{b1} and R^{b2} can combine with the nitrogen atom to which they are bound to form a morpholinyl. In certain embodiments, R^{b1} and R^{b2} can combine with the nitrogen atom to which they are bound to form a piperidinyl. In certain embodiments, R^{b1} and R^{b2} can combine with the nitrogen atom to which they are bound to form a fluorenyl. In certain embodiments, R^{b1} and R^{b2} can combine with the nitrogen atom to which they are bound to form an optionally substituted pyrazolyl.

In certain embodiments, R^{b1} is H. In certain embodiments R^{b1} is methyl. In certain embodiments, R^{b1} is ethyl. In certain embodiments, R^{b1} is n-propyl. In certain embodiments, R^{b1} is i-propyl. In certain embodiments, R^{b1} is n-butyl. In certain embodiments, R^{b1} is sec-butyl. In certain embodiments, R^{b1} is i-butyl.

In certain embodiments, R^{b2} is H. In certain embodiments R^{b2} is methyl. In certain embodiments, R^{b2} is ethyl. In certain embodiments, R^{b2} is n-propyl. In certain embodiments, R^{b2} is i-propyl. In certain embodiments, R^{b2} is n-butyl. In certain embodiments, R^{b2} is sec-butyl. In certain embodiments, R^{b2} is i-butyl.

In certain embodiments, Y¹ is OMe. In certain embodiments, Y¹ is NMe₂. In certain embodiments, Y¹ is NEt₂. In certain embodiments, Y is

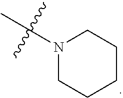 [SMILES: *N1CCCCC1]

In certain embodiments, Y¹ is

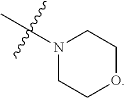 [SMILES: *N1CCOCC1]

In certain embodiments, Y¹ is

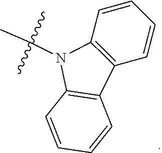 [SMILES: *n1c2ccccc2c2ccccc21]

In certain embodiments, Y¹ is

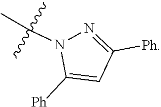 [SMILES: *n1nc(-c2ccccc2)cc1-c1ccccc1]

In certain embodiments, Y² is NMe₂. In certain embodiments, Y² is NEt₂. In certain embodiments, Y² is

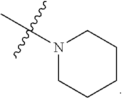 [SMILES: *N1CCCCC1]

In certain embodiments, Y² is

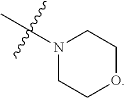 [SMILES: *N1CCOCC1]

In certain embodiments, Y² is

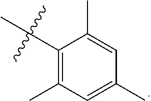

In certain embodiments, R^{c1} is methyl. In certain embodiments, R^{c1} is i-propyl. In certain embodiments, R^{c1} is diphenylmethyl.

In certain embodiments, R^{c5} is methyl. In certain embodiments, R^{c5} is i-propyl. In certain embodiments, R^{c5} is diphenylmethyl.

In certain embodiments, R^{c1} and R^{c5} are identical. In certain embodiments, R^{c1} is methyl and R^{c5} is methyl. In certain embodiments, R^{c1} is i-propyl and R^{c5} is i-propyl. In certain embodiments, R^{c1} is diphenylmethyl and R^{c5} is diphenylmethyl.

In certain embodiments, R^{c3} is H. In certain embodiments, R^{c3} is methyl.

In certain embodiments, R^{c2} is H. In certain embodiments, R^{c4} is H. In certain embodiments, R^{c2} and R^{c4} are H.

In certain embodiments, Z is

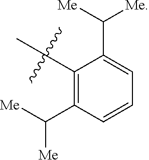 [SMILES: *c1c(C(C)C)cccc1C(C)C]

In certain embodiments, Z is

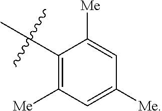 [SMILES: *c1c(C)cc(C)cc1C]

In certain embodiments, Z is

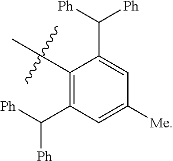 [SMILES: *c1c(C(c2ccccc2)c2ccccc2)cc(C)cc1C(c1ccccc1)c1ccccc1]

In certain embodiments, the compound of formula (IIa) or (IIb) is selected from the group consisting of:
2-(2,6-diisopropylphenyl)-5-(dimethylamino)imidazo[1,5-a]pyridin-3-ylidene gold(I) chloride;
5-(dimethylamino)-2-mesitylimidazo[1,5-a]pyridin-3-ylidene gold(I) chloride;
2-(2,6-dibenzhydryl-4-methylphenyl)-5-(dimethylamino)imidazo[1,5-a]pyridin-3-ylidene gold(I) chloride;
5-(diethylamino)-2-(2,6-diisopropylphenyl)imidazo[1,5-a]pyridin-3-ylidene gold(I) chloride;
5-(diethylamino)-2-mesitylimidazo[1,5-a]pyridin-3-ylidene gold(I) chloride;
2-(2,6-dibenzhydryl-4-methylphenyl)-5-(diethylamino)imidazo[1,5-a]pyridin-3-ylidene gold(I) chloride;
2-(2,6-diisopropylphenyl)-5-(piperidin-1-yl)imidazo[1,5-a]pyridin-3-ylidene gold(I) chloride;
2-mesityl-5-(piperidin-1-yl)imidazo[1,5-a]pyridin-3-ylidene gold(I) chloride;
2-(2,6-dibenzhydryl-4-methylphenyl)-5-(piperidin-1-yl)imidazo[1,5-a]pyridin-3-ylidene gold(I) chloride;
2-(2,6-diisopropylphenyl)-5-methoxyimidazo[1,5-a]pyridin-3-ylidene gold(I) chloride;
cinnamyl[2-(2,6-diisopropylphenyl)-5-(dimethylamino)imidazo[1,5-a]pyridine-3-ylidene]chloropalladium(II);
cinnamyl[5-(dimethylamino)-2-mesitylimidazo[1,5-a]pyridin-3-ylidene]chloropalladium(II);
cinnamyl[2-(2,6-dibenzhydryl-4-methylphenyl)-5-(dimethylamino)imidazo[1,5-a]pyridin-3-ylidene]chloropalladium(II);
cinnamyl[5-(diethylamino)-2-(2,6-diisopropylphenyl)imidazo[1,5-a]pyridin-3-ylidene]chloropalladium(II);
cinnamyl[5-(diethylamino)-2-mesitylimidazo[1,5-a]pyridin-3-ylidene]chloropalladium(II);
cinnamyl[2-(2,6-dibenzhydryl-4-methylphenyl)-5-(diethylamino)imidazo[1,5-a]pyridin-3-ylidene]chloropalladium(II);
cinnamyl[2-(2,6-diisopropylphenyl)-5-(piperidin-1-yl)imidazo[1,5-a]pyridin-3-ylidene]chloropalladium(II);
cinnamyl[2-mesityl-5-(piperidin-1-yl)imidazo[1,5-a]pyridin-3-ylidene]chloropalladium(II);
cinnamyl[2-(2,6-dibenzhydryl-4-methylphenyl)-5-(piperidin-1-yl)imidazo[1,5-a]pyridin-3-ylidene]chloropalladium(II);
cinnamyl[2-(2,6-diisopropylphenyl)-5-morpholinoimidazo[1,5-a]pyridin-3-ylidene]chloropalladium(II);
2-(2,6-diisopropylphenyl)-5-(dimethylamino)imidazo[1,5-a]pyridin-3-ylidene silver(I) chloride;
2-(2,6-diisopropylphenyl)-5-(dimethylamino)imidazo[1,5-a]pyridin-3-ylidene copper(I) chloride;
2-(2,6-diisopropylphenyl)-5-(dimethylamino)imidazo[1,5-a]pyridin-3-ylidene(cyclooctadiene)rhodium(I) chloride;
2-(2,6-diisopropylphenyl)-5-(dimethylamino)imidazo[1,5-a]pyridin-3-ylidene(cyclooctadiene)rhodium(I) triflate;
2-(2,6-diisopropylphenyl)-5-(dimethylamino)imidazo[1,5-a]pyridin-3-ylidene(bis(carbonyl))rhodium(I) triflate;
2-(2,6-diisopropylphenyl)-5-(dimethylamino)imidazo[1,5-a]pyridin-3-ylidene gold(I) bis(trifluoromethanesulfonyl)amide;
2-(2,6-diisopropylphenyl)-5-(morpholin-4-yl)imidazo[1,5-a]pyridin-3-ylidene gold(I) chloride;
2-(2,4,6-trimethylphenyl)-5-(morpholin-4-yl)imidazo[1,5-a]pyridin-3-ylidene gold(I) chloride;
2-(2,6-Diisopropylphenyl)-9-(dimethylamino)imidazo[1,5-a]quinolin-3-ylidene silver(I) chloride;
2-(2,6-Diisopropylphenyl)-9-(diethylamino)imidazo[1,5-a]quinolin-3-ylidene silver(I) chloride;
2-(2,6-Diisopropylphenyl)-9-(piperidin-1-yl)imidazo[1,5-a]quinolin-3-ylidene silver(I) chloride;
2-(2,6-Diisopropylphenyl)-9-(morpholin-4-yl)imidazo[1,5-a]quinolin-3-ylidene silver(I) chloride;
2-(2,4,6-trimethylphenyl)-9-(dimethylamino)imidazo[1,5-a]quinolin-3-ylidene silver(I) chloride;
2-(2,6-Diisopropylphenyl)-9-(dimethylamino)imidazo[1,5-a]quinolin-3-ylidene gold(I) chloride;
2-(2,6-Diisopropylphenyl)-9-(diethylamino)imidazo[1,5-a]quinolin-3-ylidene gold(I) chloride;
2-(2,6-Diisopropylphenyl)-9-(piperidin-1-yl)imidazo[1,5-a]quinolin-3-ylidene gold(I) chloride;
2-(2,6-Diisopropylphenyl)-9-(morpholin-4-yl)imidazo[1,5-a]quinolin-3-ylidene gold(I) chloride;
2-(2,4,6-trimethylphenyl)-9-(dimethylamino)imidazo[1,5-a]quinolin-3-ylidene gold(I) chloride; and
2-(2,6-Diisopropylphenyl)-9-(2,4,6-trimethylphenyl)imidazo[1,5-a]quinolin-3-ylidene gold(I) chloride.

TABLE 1
N-Heterocyclic Carbene (NHC) Ligands and Catalyst Complexes

| Cmpd | Structure | Name |
|---|---|---|
| 1 | 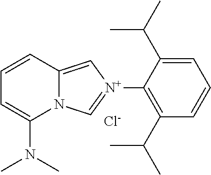 [SMILES: CC(C)c1cccc(C(C)C)c1-[n+]1cc2cccc(N(C)C)n2c1 ; [Cl-]] | 2-(2,6-diisopropylphenyl)-5- (dimethylamino)imidazo[1,5- a]pyridin-2-ium chloride |
| 2 | 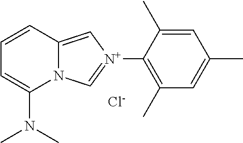 [SMILES: Cc1cc(C)c(-[n+]2cc3cccc(N(C)C)n3c2)c(C)c1 ; [Cl-]] | 5-(dimethylamino)-2- mesitylimidazo[1,5-a]pyridin-2-ium chloride |
| 3 | 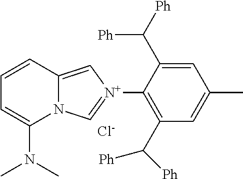 [SMILES: Cc1cc(C(c2ccccc2)c2ccccc2)c(-[n+]2cc3cccc(N(C)C)n3c2)c(C(c2ccccc2)c2ccccc2)c1 ; [Cl-]] | 2-(2,6-dibenzhydryl-4- methylphenyl)-5- (dimethylamino)imidazo[1,5- a]pyridin-2-ium chloride |
| 4 | 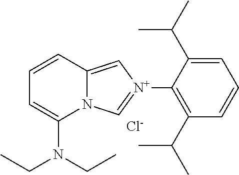 [SMILES: CCN(CC)c1cccc2c[n+](-c3c(C(C)C)cccc3C(C)C)cn12 ; [Cl-]] | 5-(diethylamino)-2-(2,6- diisopropylphenyl)imidazo[1,5- a]pyridin-2-ium chloride |
| 5 | 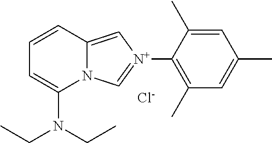 [SMILES: CCN(CC)c1cccc2c[n+](-c3c(C)cc(C)cc3C)cn12 ; [Cl-]] | 5-(diethylamino)-2- mesitylimidazo[1,5-a]pyridin-2-ium chloride |
| 6 | 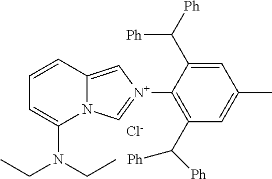 [SMILES: CCN(CC)c1cccc2c[n+](-c3c(C(c4ccccc4)c4ccccc4)cc(C)cc3C(c3ccccc3)c3ccccc3)cn12 ; [Cl-]] | 2-(2,6-dibenzhydryl-4- methylphenyl)-5- (diethylamino)imidazo[1,5-a]pyridin- 2-ium chloride |
| 7 | 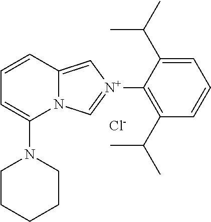 [SMILES: CC(C)c1cccc(C(C)C)c1-[n+]1cc2cccc(N3CCCCC3)n2c1 ; [Cl-]] | 2-(2,6-diisopropylphenyl)-5- (piperidin-1-yl)imidazo[1,5- a]pyridin-2-ium chloride |
| 8 | 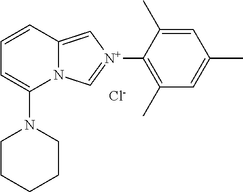 [SMILES: Cc1cc(C)c(-[n+]2cc3cccc(N4CCCCC4)n3c2)c(C)c1 ; [Cl-]] | 2-mesityl-5-(piperidin-1- yl)imidazo[1,5-a]pyridin-2-ium chloride |
| 9 | 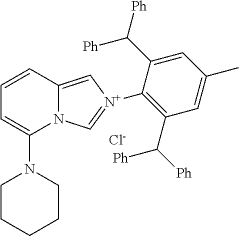 [SMILES: Cc1cc(C(c2ccccc2)c2ccccc2)c(-[n+]2cc3cccc(N4CCCCC4)n3c2)c(C(c2ccccc2)c2ccccc2)c1 ; [Cl-]] | 2-(2,6-dibenzhydryl-4- methylphenyl)-5-(piperidin-1- yl)imidazo[1,5-a]pyridin-2-ium chloride |
| 10 | 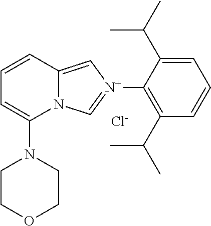 [SMILES: CC(C)c1cccc(C(C)C)c1-[n+]1cc2cccc(N3CCOCC3)n2c1 ; [Cl-]] | 2-(2,6-diisopropylphenyl)-5- morpholinoimidazo[1,5-a]pyridin-2- ium chloride |
| 11 | 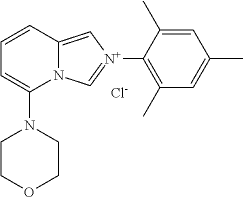 [SMILES: Cc1cc(C)c(-[n+]2cc3cccc(N4CCOCC4)n3c2)c(C)c1 ; [Cl-]] | 2-mesityl-5-morpholinoimidazo[1,5- a]pyridin-2-ium chloride |
| 12 | 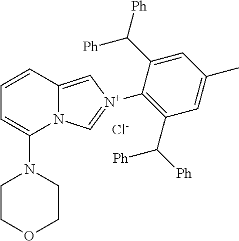 [SMILES: Cc1cc(C(c2ccccc2)c2ccccc2)c(-[n+]2cc3cccc(N4CCOCC4)n3c2)c(C(c2ccccc2)c2ccccc2)c1 ; [Cl-]] | 2-(2,6-dibenzhydryl-4- methylphenyl)-5- morpholinoimidazo[1,5-a]pyridin-2- ium chloride |
| 13 | 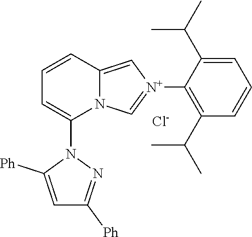 [SMILES: CC(C)c1cccc(C(C)C)c1-[n+]1cc2cccc(-n3nc(-c4ccccc4)cc3-c3ccccc3)n2c1 ; [Cl-]] | 2-(2,6-diisopropylphenyl)-5-(3,5- diphenyl-1H-pyrazol-1- yl)imidazo[1,5-a]pyridin-2-ium chloride |
| 14 | 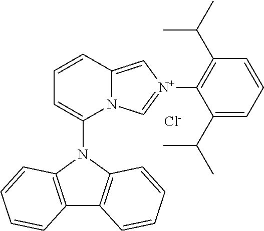 [SMILES: CC(C)c1cccc(C(C)C)c1-[n+]1cc2cccc(-n3c4ccccc4c4ccccc43)n2c1 ; [Cl-]] | 5-(9H-carbazol-9-yl)-2-(2,6- diisopropylphenyl)imidazo[1,5- a]pyridin-2-ium chloride |
| 15 | 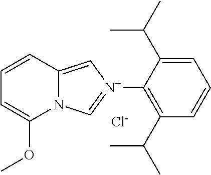 [SMILES: COc1cccc2c[n+](-c3c(C(C)C)cccc3C(C)C)cn12 ; [Cl-]] | 2-(2,6-diisopropylphenyl)-5- methoxyimidazo[1,5-a]pyridin-2-ium chloride |
| 16 | 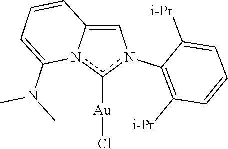 | 2-(2,6-diisopropylphenyl)-5- (dimethylamino)imidazo[1,5- a]pyridin-3-ylidene gold(I) chloride |
| 17 | 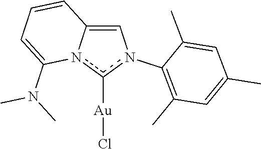 | 5-(dimethylamino)-2- mesitylimidazo[1,5-a]pyridin-3- ylidene gold(I) chloride |
| 18 | 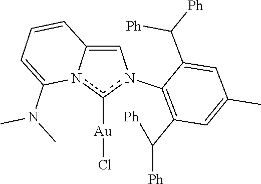 | 2-(2,6-dibenzhydryl-4- methylphenyl)-5- (dimethylamino)imidazo[1,5- a]pyridin-3-ylidene gold(I) chloride |
| 19 | 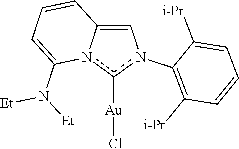 | 5-(diethylamino)-2-(2,6- diisopropylphenyl)imidazo[1,5- a]pyridin-3-ylidene gold(I) chloride |
| 20 | 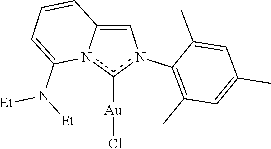 | 5-(diethylamino)-2- mesitylimidazo[1,5-a]pyridin-3- ylidene gold(I) chloride |
| 21 | 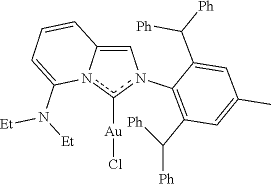 | 2-(2,6-dibenzhydryl-4- methylphenyl)-5- (diethylamino)imidazo[1,5-a]pyridin- 3-ylidene gold(I) chloride |
| 22 | 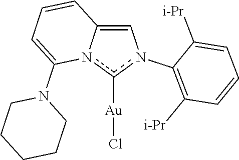 | 2-(2,6-diisopropylphenyl)-5- (piperidin-1-yl)imidazo[1,5- a]pyridin-3-ylidene gold(I) chloride |
| 23 | 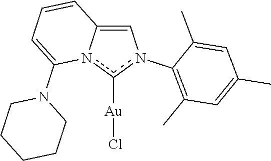 | 2-mesityl-5-(piperidin-1- yl)imidazo[1,5-a]pyridin-3-ylidene gold(I) chloride |
| 24 | 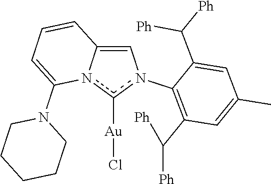 | 2-(2,6-dibenzhydryl-4- methylphenyl)-5-(piperidin-1- yl)imidazo[1,5-a]pyridin-3-ylidene gold(I) chloride |
| 25 | 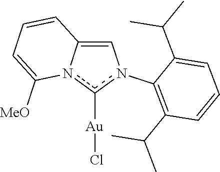 | 2-(2,6-diisopropylphenyl)-5- methoxyimidazo[1,5-a]pyridin-3- ylidene gold(I) chloride |
| 26 | 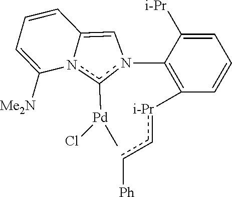 | cinnamyl[2-(2,6-diisopropylphenyl)- 5-(dimethylamino)imidazo[1,5- a]pyridine-3- ylidene]chloropalladium(II) |
| 27 | 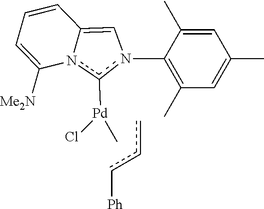 | cinnamyl[5-(dimethylamino)-2- mesitylimidazo[1,5-a]pyridin-3- ylidene]chloropalladium(II) |
| 28 | 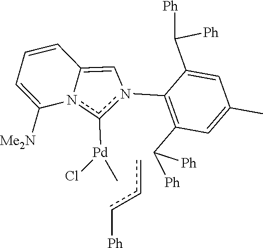 | cinnamyl[2-(2,6-dibenzhydryl-4- methylphenyl)-5- (dimethylamino)imidazo[1,5- a]pyridin-3- ylidene]chloropalladium(II) |
| 29 | 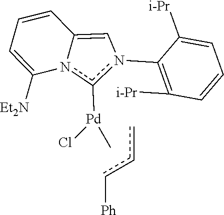 | cinnamyl[5-(diethylamino)-2-(2,6- diisopropylphenyl)imidazo[1,5- a]pyridin-3- ylidene]chloropalladium(II) |
| 30 | 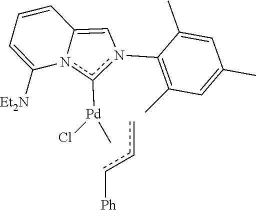 | cinnamyl[5-(diethylamino)-2- mesitylimidazo[1,5-a]pyridin-3- ylidene]chloropalladium(II) |
| 31 | 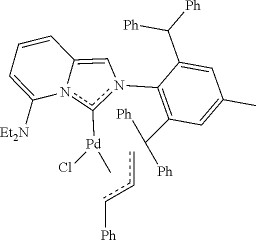 | cinnamyl[2-(2,6-dibenzhydryl-4- methylphenyl)-5- (diethylamino)imidazo[1,5-a]pyridin- 3-ylidene]chloropalladium(II) |
| 32 | 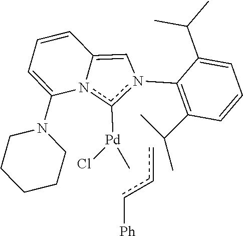 | cinnamyl[2-(2,6-diisopropylphenyl)- 5-(piperidin-1-yl)imidazo[1,5- a]pyridin-3- ylidene]chloropalladium(II) |
| 33 | 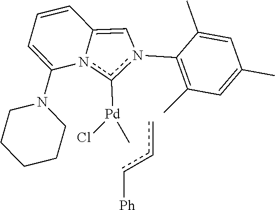 | cinnamyl[2-mesityl-5-(piperidin-1- yl)imidazo[1,5-a]pyridin-3- ylidene]chloropalladium(II) |
| 34 | 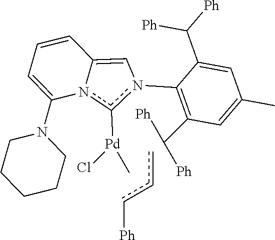 | cinnamyl[2-(2,6-dibenzhydryl-4- methylphenyl)-5-(piperidin-1- yl)imidazo[1,5-a]pyridin-3- ylidene]chloropalladium(II) |
| 35 | 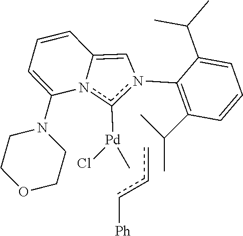 | cinnamyl[2-(2,6-diisopropylphenyl)- 5-morpholinoimidazo[1,5-a]pyridin- 3-ylidene]chloropalladium(II) |
| 36 | 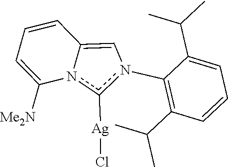 | 2-(2,6-diisopropylphenyl)-5- (dimethylamino)imidazo[1,5- a]pyridin-3-ylidene silver(I) chloride |
| 37 | 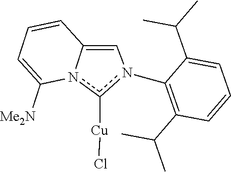 | 2-(2,6-diisopropylphenyl)-5- (dimethylamino)imidazo[1,5- a]pyridin-3-ylidene copper(I) chloride |
| 38 | 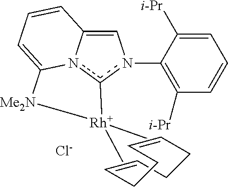 | 2-(2,6-diisopropylphenyl)-5- (dimethylamino)imidazo[1,5- a]pyridin-3- ylidene(cyclooctadiene)rhodium(I) chloride |
| 39 | 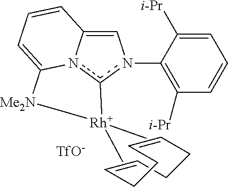 | 2-(2,6-diisopropylphenyl)-5- (dimethylamino)imidazo[1,5- a]pyridin-3- ylidene(cyclooctadiene)rhodium(I) triflate |
| 40 | 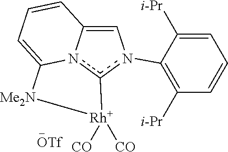 | 2-(2,6-diisopropylphenyl)-5- (dimethylamino)imidazo[1,5- a]pyridin-3- ylidene(bis(carbonyl))rhodium(I) triflate |
| 41 | 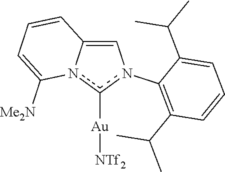 | 2-(2,6-diisopropylphenyl)-5- (dimethylamino)imidazo[1,5- a]pyridin-3-ylidene gold(I) bis(trifluoromethanesulfonyl)amide |
| 42 | 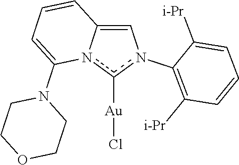 | 2-(2,6-diisopropylphenyl)-5- (morpholin-4-yl)imidazo[1,5- a]pyridin-3-ylidene gold(I) chloride |
| 43 | 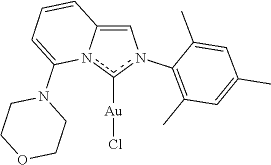 | 2-(2,4,6-trimethylphenyl)-5- (morpholin-4-yl)imidazo[1,5- a]pyridin-3-ylidene gold(I) chloride |
| 44 | 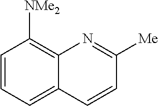 [SMILES: Cc1ccc2cccc(N(C)C)c2n1] | N,N,2-trimethylquinolin-8-amine |
| 45 | 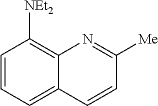 | N,N-diethyl-2-methylquinolin-8- amine |
| 46 | 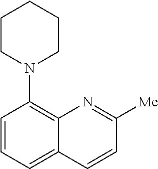 [SMILES: Cc1ccc2cccc(N3CCCCC3)c2n1] | 2-methyl-8-(piperidin-1-yl)quinoline |
| 47 | 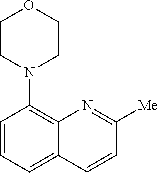 [SMILES: Cc1ccc2cccc(N3CCOCC3)c2n1] | 4-(2-methylquinolin-8-yl)morpholine |
| 48 | 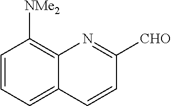 [SMILES: CN(C)c1cccc2ccc(C=O)nc12] | 8-(dimethylamino)quinoline-2- carbaldehyde |
| 49 | 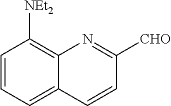 | 8-(diethylamino)quinoline-2- carbaldehyde |
| 50 | 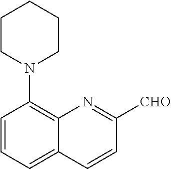 [SMILES: O=Cc1ccc2cccc(N3CCCCC3)c2n1] | 8-(piperidin-1-yl)quinoline-2- carbaldehyde |
| 51 | 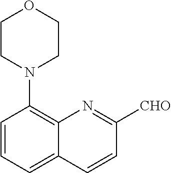 [SMILES: O=Cc1ccc2cccc(N3CCOCC3)c2n1] | 8-morpholinoquinoline-2- carbaldehyde |
| 52 | 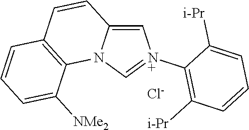 [SMILES: CC(C)c1cccc(C(C)C)c1-[n+]1cc2ccc3cccc(N(C)C)c3n2c1 ; [Cl-]] | 2-(2,6-Diisopropylphenyl)-9- (dimethylamino)imidazo[1,5- a]quinolin-2-ium chloride |
| 53 | 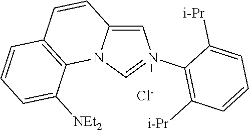 | 2-(2,6-Diisopropylphenyl)-9- (diethylamino)imidazo[1,5- a]quinolin-2-ium chloride |
| 54 | 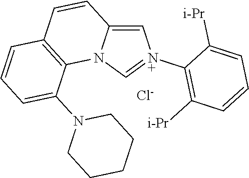 [SMILES: CC(C)c1cccc(C(C)C)c1-[n+]1cc2ccc3cccc(N4CCCCC4)c3n2c1 ; [Cl-]] | 2-(2,6-Diisopropylphenyl)-9- (piperidin-1-yl)imidazo[1,5- a]quinolin-2-ium chloride |
| 55 | 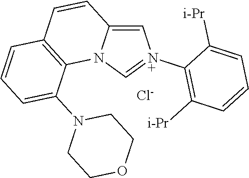 [SMILES: CC(C)c1cccc(C(C)C)c1-[n+]1cc2ccc3cccc(N4CCOCC4)c3n2c1 ; [Cl-]] | 2-(2,6-Diisopropylphenyl)-9- morpholinoimidazo[1,5-a]quinolin-2- ium chloride |
| 56 | 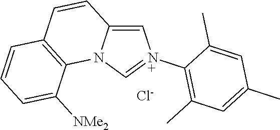 [SMILES: Cc1cc(C)c(-[n+]2cc3ccc4cccc(N(C)C)c4n3c2)c(C)c1 ; [Cl-]] | 9-(Dimethylamino)-2- mesitylimidazo[1,5-a]quinolin-2-ium chloride |
| 57 | 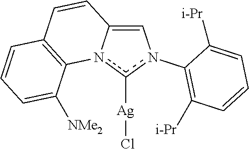 | 2-(2,6-Diisopropylphenyl)-9- (dimethylamino)imidazo[1,5- a]quinolin-3-ylidene silver(I) chloride |
| 58 | 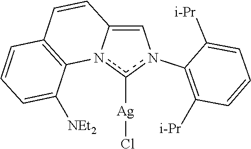 | 2-(2,6-Diisopropylphenyl)-9- (diethylamino)imidazo[1,5- a]quinolin-3-ylidene silver(I) chloride |
| 59 | 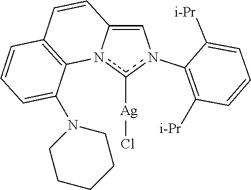 | 2-(2,6-Diisopropylphenyl)-9- (piperidin-1-yl)imidazo[1,5- a]quinolin-3-ylidene silver(I) chloride |
| 60 | 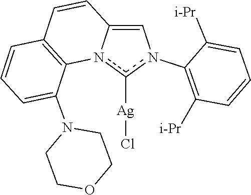 | 2-(2,6-Diisopropylphenyl)-9- (morpholin-4-yl)imidazo[1,5- a]quinolin-3-ylidene silver(I) chloride |
| 61 | 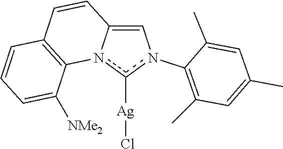 | 2-(2,4,6-trimethylphenyl)-9- (dimethylamino)imidazo[1,5- a]quinolin-3-ylidene silver(I) chloride |
| 62 | 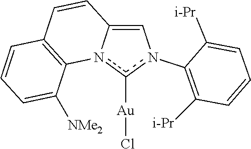 | 2-(2,6-Diisopropylphenyl)-9- (dimethylamino)imidazo[1,5- a]quinolin-3-ylidene gold(I) chloride |
| 63 | 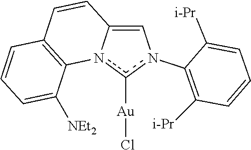 | 2-(2,6-Diisopropylphenyl)-9- (diethylamino)imidazo[1,5- a]quinolin-3-ylidene gold(I) chloride |
| 64 | 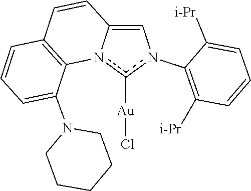 | 2-(2,6-Diisopropylphenyl)-9- (piperidin-1-yl)imidazo[1,5- a]quinolin-3-ylidene gold(I) chloride |
| 65 | 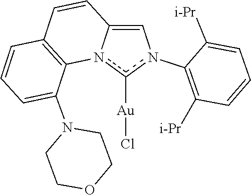 | 2-(2,6-Diisopropylphenyl)-9- (morpholin-4-yl)imidazo[1,5- a]quinolin-3-ylidene gold(I) chloride |
| 66 | 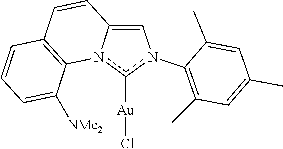 | 2-(2,4,6-trimethylphenyl)-9- (dimethylamino)imidazo[1,5- a]quinolin-3-ylidene gold(I) chloride |
| 67 | 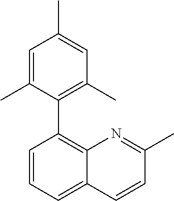 | 8-mesityl-2-methylquinoline |
| 68 | 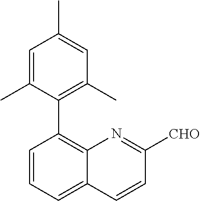 [SMILES: Cc1cc(C)c(-c2cccc3ccc(C=O)nc23)c(C)c1] | 8-mesitylquinoline-2-carbaldehyde |
| 69 | 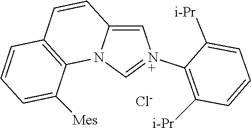 | 2-(2,6-Diisopropylphenyl)-9-(2,4,6- trimethylphenyl)imidazo[1,5- a]quinolin-2-ium chloride |
| 70 | 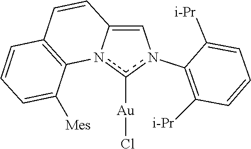 | 2-(2,6-Diisopropylphenyl)-9-(2,4,6- trimethylphenyl)imidazo[1,5- a]quinolin-3-ylidene gold(I) chloride |
| A1 | 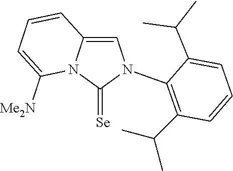 [SMILES: CC(C)c1cccc(C(C)C)c1-n1cc2cccc(N(C)C)n2c1=[Se]] | 2-(2,6-diisopropylphenyl)-5- (dimethylamino)imidazo[1,5- a]pyridine-3(2H)-selenone |
| A2 | 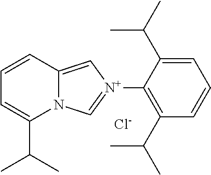 [SMILES: CC(C)c1cccc(C(C)C)c1-[n+]1cc2cccc(C(C)C)n2c1 ; [Cl-]] | 2-(2,6-Diisopropylphenyl)-5- isopropylimidazo[1,5-a]pyridin-2- ium chloride |
| A3 | 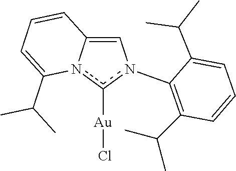 | 2-(2,6-Diisopropylphenyl)-5- isopropylimidazo[1,5-a]pyridin-3- ylidene gold(I) chloride |
| A4 | 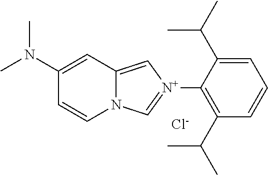 [SMILES: CC(C)c1cccc(C(C)C)c1-[n+]1cc2cc(N(C)C)ccn2c1 ; [Cl-]] | 2-(2,6-Diisopropylphenyl)-7- (dimethylamino)imidazo[1,5- a]pyridin-2-ium chloride |
| A5 | 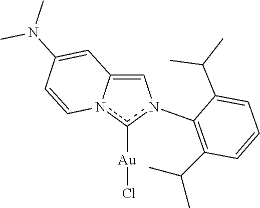 | 2-(2,6-Diisopropylphenyl)-7- (dimethylamino)imidazo[1,5- a]pyridin-3-ylidene gold(I) chloride |
| A6 | 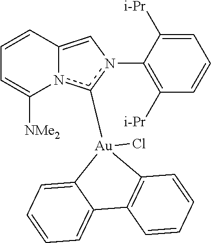 | (2-(2,6-Diisopropylphenyl)-5- (dimethylamino)imidazo[1,5- a]pyridin-3-ylidene)(2,2′-biphenyl) gold(III) chloride |

### Methods

### Ligand Synthesis

The present disclosure further provides a method of preparing the compound formula (Ia), the method comprising:
contacting a compound of formula (A):

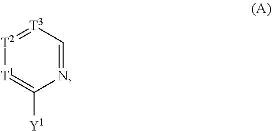

a compound of formula (B):

H₂N—Z (B), and
formaldehyde in the presence of an acid to form the compound of formula (Ia).

In certain embodiments, the formaldehyde is paraformaldehyde:

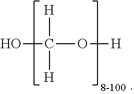

In certain embodiments, the contacting occurs in the presence of a solvent. In certain embodiments, the solvent is ethanol.

In certain embodiments, the contacting occurs at room temperature. In certain embodiments, the temperature of contacting is elevated to a second temperature selected from the group consisting of about 40, 45, 50, 55, 60, 65, 70, 75, and about 80° C.

In certain embodiments, the contacting for a period of time selected from the group consisting of about 12, 13, 14, 15, 16, 17, 18, 19, 20, 21, 22, 23, 24, 25, 26, 27, 28, 29, 30, 31, 32, 33, 34, 35, and about 36 h.

The present disclosure further provides a method of preparing 2-(2,6-Diisopropylphenyl)-5-(dimethylamino)imidazo[1,5-a]pyridin-2-ium chloride) (1):

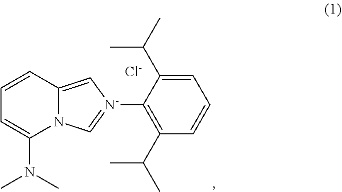

the method comprising reacting (E)-6-(((2,6-diisopropylphenyl)imino)methyl)-N,N-dimethylpyridin-2-amine (Z):

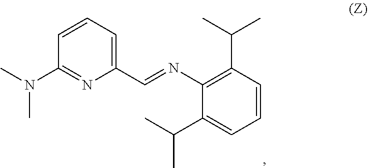 [SMILES: CC(C)c1cccc(C(C)C)c1/N=C/c1cccc(N(C)C)n1]

and paraformaldehyde so as to generate a first reaction system comprising (1).

In certain embodiments, the reaction of (Z) and paraformaldehyde is performed in the presence of a solvent. In certain embodiments, the solvent is EtOH.

In certain embodiments, the reaction further comprises hydrochloric acid (HCl).

In certain embodiments, the reaction of (Z) and paraformaldehyde is performed at a temperature selected from the group consisting of about 50, 55, 60, 65, 70, 75, 80, 85, and 90° C.

In certain embodiments, the (Z) is prepared by reacting 6-(dimethylamino)picolinaldehyde (Y):

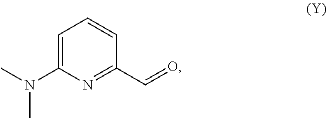

and 2,6-diisopropylaniline (X):

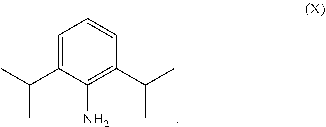

In certain embodiments, the reaction of (Y) and (X) is performed in the presence of a solvent. In certain embodiments, the solvent is EtOH.

In certain embodiments, the reaction of (Y) and (X) is performed at a temperature selected from the group consisting of about 70, 75, 80, 85, 90, 95, and 100° C.

In certain embodiments, the (Y) is prepared by reacting 6-bromo-N,N-dimethylpyridin-2-amine (D):

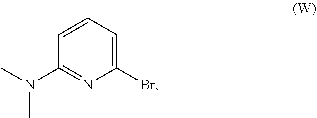

and an organolithium reagent to form a lithiated intermediate,
and contacting the lithiated intermediate with a formylating reagent.

In certain embodiments, the reaction of (W) and the organolithium reagent is performed in the presence of a solvent.

In certain embodiments, the solvent is tetrahydrofuran (THF).

In certain embodiments, the organolithium reagent is selected from the group consisting of n-butyllithium (n-BuLi), sec-butyllithium (s-BuLi), t-butyllithium (t-BuLi), and phenyllithium (PhLi).

In certain embodiments, the reaction of (W) and the organolithium reagent is performed at about −78° C.

In certain embodiments, the reaction of the lithiated intermediate and the formylating reagent is performed in the presence of a solvent. In certain embodiments, the solvent is tetrahydrofuran (THF).

In certain embodiments, the formylating agent is dimethylformamide (DMF).

In certain embodiments, the contacting occurs at a temperature of about −78° C.

In certain embodiments, the (W) is prepared by reacting (V):

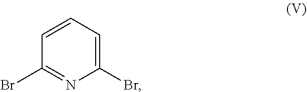

and dimethylamine (HNMe₂), in the presence of a base.

In certain embodiments, the reaction of (V) and dimethylamine is performed in the presence of a solvent.

In certain embodiments, the solvent is acetonitrile (ACN).

In certain embodiments, the base is K₂CO₃.

In certain embodiments, the reaction of (V) and dimethylamine is performed at a temperature selected from the group consisting of about 80, 85, 90, 95, 100, 105, 110, 115, and 120° C.

### Catalysis

The present disclosure provides a method of promoting a reaction (coupling) between a first reagent and an aryl iodide (i.e., promoting arylation of a first reagent). In certain embodiments, the method comprises contacting the first reagent and the aryl iodide in the presence of the N-heterocyclic carbene complex of the present disclosure.

In certain embodiments, the contacting occurs in the presence of a Lewis acid.

In certain embodiments, M is Au in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the first reagent is selected from the group consisting of optionally substituted C₆-C₁₀ aryl and optionally substituted C₂-C₁₀ heteroaryl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)(C₁-C₆ alkyl), C(═O)NH₂, C(═O)NH(C₁-C₆ alkyl), and C(═O)N(C₁-C₆ alkyl)₂.

In certain embodiments, the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the Lewis acid is AgNTf₂. In certain embodiments, the Lewis acid is sodium tetrakis[3,5-bis(trifluoromethyl)phenyl]borate (NaBAr^{F}). In certain embodiments, the Lewis acid is AgSbF₆.

In certain embodiments, the contacting occurs in the presence of a solvent. In certain embodiments, the solvent is MeOH.

In certain embodiments, the contacting occurs at a temperature selected from the group consisting of about 40, 45, 50, 55, 60, 65, 70, 75, and about 80° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

The present disclosure further provides a method of promoting a reaction (coupling) between an aniline and an aryl iodide (i.e., promoting arylation of an aniline), the method comprising contacting the aniline and the aryl iodide in the presence of the NHC complex of the present disclosure.

In certain embodiments, the contacting occurs in the presence of a Lewis acid.

In certain embodiments, M is Au in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the aniline is selected from the group consisting of optionally substituted C₆-C₁₀ aryl and C₂-C₁₀ heteroaryl, wherein the aryl or heteroaryl is substituted with at least one NH₂, and wherein each optional substituent is at least one selected from the group consisting of of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the Lewis acid is AgNTf₂. In certain embodiments, the Lewis acid is sodium tetrakis[3,5-bis(trifluoromethyl)phenyl]borate (NaBAr^{F}). In certain embodiments, the Lewis acid is AgSbF₆.

In certain embodiments, the contacting occurs in the presence of a solvent. In certain embodiments, the solvent is MeOH.

In certain embodiments, the contacting occurs at a temperature selected from the group consisting of about 40, 45, 50, 55, 60, 65, 70, 75, and about 80° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

The present disclosure further provides a method of promoting a hydroamination reaction between an alkyne and an amine, the method comprising contacting the alkyne and the amine in the presence of the NHC complex of the present disclosure.

In certain embodiments, the contacting occurs in the presence of a Lewis acid.

In certain embodiments, M is Au in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the alkyne is selected from the group consisting of optionally substituted C₂-C₁₂ alkynyl, optionally substituted C₅-C₁₂ aralkynyl, and optionally substituted C₄-C₁₂ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the amine is selected from the group consisting of optionally substituted C₄-C₁₂ heterocycloalkyl comprising at least one secondary amine, H₂N—NH₂, H₂N—N(optionally substituted C₁-C₆ alkyl)₂, H₂N—N(optionally substituted C₁-C₆ alkyl)(optionally substituted C₄-C₁₀ aryl), H₂N—N(optionally substituted C₄-C₁₀ aryl)₂, H₂N(optionally substituted C₁-C₆ alkyl), H₂N(optionally substituted C₄-C₁₀ aryl), HN(optionally substituted C₁-C₆ alkyl)(optionally substituted C₄-C₁₀ aryl), HN(optionally substituted C₄-C₁₀ aryl)₂, and NH(optionally substituted C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, halogen, CN, NO₂, C(═O)NH₂, C(═O)NH(C₁-C₆ alkyl), and C(═O)N(C₁-C₆ alkyl)₂.

In certain embodiments, the Lewis acid is AgNTf₂. In certain embodiments, the Lewis acid is sodium tetrakis[3,5-bis(trifluoromethyl)phenyl]borate (NaBAr^{F}). In certain embodiments, the Lewis acid is AgSbF₆.

In certain embodiments, the contacting occurs in the presence of a solvent. In certain embodiments, the solvent is toluene.

In certain embodiments, the contacting occurs at a temperature selected from the group consisting of about 90, 91, 92, 93, 94, 95, 96, 97, 98, 99, 100, 101, 102, 103, 104, 105, 106, 107, 108, 109, and about 110° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

In certain embodiments, the contacting of the alkyne and amine provides an imine intermediate. In certain embodiments, a reduction reaction is promoted by contacting the imine intermediate and a reducing agent. In certain embodiments, the reducing agent is sodium triacetoxyborohydride (NaBH(OAc)₃).

The present disclosure further provides method of promoting hydration of an alkyne, the method comprising contacting the alkyne and water in the presence of the NHC complex of the present disclosure.

In certain embodiments, the contacting occurs in the presence of a Lewis acid.

In certain embodiments, M is Au in the NHC complex of the present disclosure.

In certain embodiments, the NHC complex of the present disclosure is present in an amount selected from the group consisting of about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 and about 1 mol %.

In certain embodiments, the alkyne is selected from the group consisting of optionally substituted C₂-C₁₂ alkynyl, optionally substituted C₈-C₁₂ aralkynyl, and optionally substituted C₄-C₁₂ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the Lewis acid is AgNTf₂. In certain embodiments, the Lewis acid is sodium tetrakis[3,5-bis(trifluoromethyl)phenyl]borate (NaBAr^{F}). In certain embodiments, the Lewis acid is AgSbF₆.

In certain embodiments, the contacting occurs in the presence of a solvent. In certain embodiments, the solvent is a mixture of 1,4-dioxane and water. In certain embodiments, the mixture of 1,4-dioxane and water has a ratio selected from the group consisting of about 10:1, 9:1, 8:1, 7:1, 6:1, 5:1, 4:1, 3:1, 2:1, 1:1, 0.9:1, 0.8:1, 0.7:1, 0.6:1, 0.5:1, 0.4:1, 0.3:1, 0.2:1, and about 0.1:1 (1,4-dioxane:water).

In certain embodiments, the contacting occurs at a temperature selected from the group consisting of about 80, 81, 82, 83, 84, 85, 86, 87, 88, 89, 90, 91, 92, 93, 94, 95, 96, 97, 98, 99 and about 100° C.

In certain embodiments, the contacting occurs for a period of time selected from the group consisting of about 8, 9, 10, 11, 12, 13, 14, 15, 16, 17, 18, 19, 20, 21, 22, 23, and about 24.

The present disclosure further provides a method of promoting a reaction (coupling) between an aryl chloride and a lithium amide (i.e., promoting arylation of an amide), the method comprising contacting the aryl chloride and the lithium amide in the presence of the NHC complex of the present disclosure.

In certain embodiments, M is Pd in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the lithium amide is prepared by contacting an amine with a second lithium amide. In certain embodiments, the second lithium amide is lithium hexamethyldisilazide (LiHMDS).

In certain embodiments, the amine is selected from the group consisting of optionally substituted C₄-C₁₂ heterocycloalkyl comprising at least one secondary amine, H₂N—NH₂, H₂N—N(optionally substituted C₁-C₆ alkyl)₂, H₂N—N(optionally substituted C₁-C₆ alkyl)(optionally substituted C₄-C₁₀ aryl), H₂N—N(optionally substituted C₄-C₁₀ aryl)₂, H₂N(optionally substituted C₁-C₆ alkyl), H₂N(optionally substituted C₄-C₁₀ aryl), HN(optionally substituted C₁-C₆ alkyl)(optionally substituted C₄-C₁₀ aryl), HN(optionally substituted C₄-C₁₀ aryl)₂, and NH(optionally substituted C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, halogen, CN, NO₂, C(═O)NH₂, C(═O)NH(C₁-C₆ alkyl), and C(═O)N(C₁-C₆ alkyl)₂.

In certain embodiments, the aryl chloride is selected from the group consisting of optionally substituted C₆-C₁₀ aryl and optionally substituted C₂-C₁₀ heteroaryl, wherein the aryl or heteroaryl is substituted with at least one chlorine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the contacting occurs in the presence of a solvent. In certain embodiments, the solvent is 1,4-dioxane.

In certain embodiments, the contacting occurs at a temperature selected from the group consisting of about 80, 81, 82, 83, 84, 85, 86, 87, 88, 89, 90, 91, 92, 93, 94, 95, 96, 97, 98, 99 and about 100° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

The present disclosure further provides a method of promoting a reaction (coupling) between an aryl chloride and an arylmagnesium halide, the method comprising contacting the aryl chloride and the arylmagnesium halide in the presence of the NHC complex of the present disclosure.

In certain embodiments, M is Pd in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the aryl chloride is selected from the group consisting of optionally substituted C₆-C₁₀ aryl and optionally substituted C₂-C₁₀ heteroaryl, wherein the aryl or heteroaryl is substituted with at least one chlorine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, and NO₂.

In certain embodiments, the arylmagnesium halide is selected from the group consisting of optionally substituted C₆-C₁₀ aryl and optionally substituted C₂-C₁₀ heteroaryl, wherein the aryl or heteroaryl is substituted with a magnesium halide moiety, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, and NO₂.

In certain embodiments, the contacting occurs in the presence of a solvent. In certain embodiments, the solvent is 1,4-dioxane.

In certain embodiments, the contacting occurs at a temperature selected from the group consisting of about 80, 81, 82, 83, 84, 85, 86, 87, 88, 89, 90, 91, 92, 93, 94, 95, 96, 97, 98, 99 and about 100° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

The present disclosure further provides a method of promoting a reaction (coupling) between an aroyl chloride and an aryl boronic acid (i.e., promoting aroylation of an aryl reagent), the method comprising contacting the aroyl chloride and the aryl boronic acid in the presence of the NHC complex of the present disclosure and a base.

In certain embodiments, M is Pd in the NHC complex of the present disclosure.

In certain embodiments, the NHC complex of the present disclosure is present in an amount ranging from about 0.1 to about 10 mol %.

In certain embodiments, the aroyl chloride is selected from the group consisting of optionally substituted C₆-C₁₀ aryl and optionally substituted C₂-C₁₀ heteroaryl, wherein the aryl or heteroaryl is substituted with a C(═O)C₁ moiety, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, CN, NO₂, C(═O)O(C₁-C₆ alkyl), and C(═O)N(C₁-C₆ alkyl)₂.

In certain embodiments, the aryl boronic acid is selected from the group consisting of optionally substituted C₆-C₁₀ aryl boronic acid and optionally substituted C₄-C₁₀ heteroaryl boronic acid, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the base is K₂CO₃.

In certain embodiments, the reaction occurs in the presence of a solvent. In certain embodiments, the solvent is 1,4-dioxane. In certain embodiments, the contacting occurs at a temperature selected from the group consisting of about 80, 81, 82, 83, 84, 85, 86, 87, 88, 89, 90, 91, 92, 93, 94, 95, 96, 97, 98, 99 and about 100° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

The present disclosure further provides a method of promoting a reaction between an aryl iodide, an aniline, and carbon monoxide (CO), the method comprising contacting the aryl iodide, the aniline, and the CO in the presence of the NHC complex of the present disclosure and a base.

In certain embodiments, M is Pd in the NHC complex of the present disclosure.

In certain embodiments, the NHC complex of the present disclosure is present in an amount selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 mol %.

In certain embodiments, the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the aniline is selected from the group consisting of optionally substituted C₆-C₁₀ aryl and C₂-C₁₀ heteroaryl, wherein the aryl or heteroaryl is substituted with at least one NH₂, and wherein each optional substituent is at least one selected from the group consisting of of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

In certain embodiments, the CO has a pressure selected from the group consisting of about 0.1, 0.2 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and about 10 atm.

In certain embodiments, the base is K₃PO₄.

In certain embodiments, the contacting occurs in the presence of a solvent. In certain embodiments, the solvent is toluene.

In certain embodiments, the contacting occurs at a temperature selected from the group consisting of about 90, 91, 92, 93, 94, 95, 96, 97, 98, 99, 100, 101, 102, 103, 104, 105, 106, 107, 108, 109, and about 110° C.

In certain embodiments, the contacting occurs for a period of time selected from the group consisting of about 8, 9, 10, 11, 12, 13, 14, 15, 16, 17, 18, 19, 20, 21, 22, 23, and about 24 h.

### EXAMPLES

Various embodiments of the present application can be better understood by reference to the following Examples which are offered by way of illustration. The scope of the present application is not limited to the Examples given herein.

### Example 1: Synthesis of Imidazo[1,5-a]pyridine N-Heterocyclic Carbene (NHC) Ligands

### Synthesis of 2-(2,6-Diisopropylphenyl)-5-(dimethylamino)imidazo[1,5-a]pyridin-2-ium chloride (1)

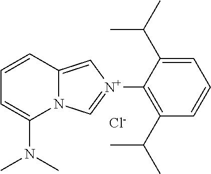 [SMILES: CC(C)c1cccc(C(C)C)c1-[n+]1cc2cccc(N(C)C)n2c1 ; [Cl-]]

An oven-dried 100 mL round-bottomed flask equipped with a stir bar was charged with 6-(dimethylamino)picolinaldehyde (1.5 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M, dioxane, 5.0 mL, 2.0 equiv) was added dropwise into the reaction at room temperature. The mixture was stirred at 70° C. for 24 hours. After the indicated time, the reaction mixture was cooled down to room temperature and concentrated. The residue was purified by chromatography on silica gel (CH₂Cl₂/MeOH=20/1) to afford the title product (3.08 g, 86%).

¹H NMR (500 MHz, CDCl₃) δ 9.85 (s, 1H), 8.41 (d, J=1.5 Hz, 1H), 8.02 (d, J=9.2 Hz, 1H), 7.57 (t, J=7.9 Hz, 1H), 7.38 (dd, J=9.1, 7.5 Hz, 1H), 7.34 (d, J=7.9 Hz, 2H), 6.70 (d, J=7.3 Hz, 1H), 3.00 (s, 6H), 2.15 (dt, J=13.6, 6.8 Hz, 2H), 1.22 (d, J=6.8 Hz, 6H), 1.19 (d, J=6.8 Hz, 6H). ¹³C NMR (126 MHz, CDCl₃) δ 144.99, 144.18, 132.38, 132.05, 130.82, 127.30, 124.58, 124.03, 117.22, 113.57, 105.10, 41.98, 28.78, 24.62, 24.28. HRMS calcd for C₂₁H₂₈N₃⁺ (M−Cl⁻) 322.2278, found 322.2281.

### Synthesis of 5-(Dimethylamino)-2-mesitylimidazo[1,5-a]pyridin-2-ium chloride (2)

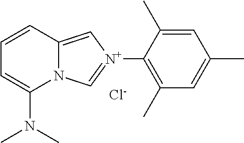 [SMILES: Cc1cc(C)c(-[n+]2cc3cccc(N(C)C)n3c2)c(C)c1 ; [Cl-]]

An oven-dried 100 mL round-bottomed flask equipped with a stir bar was charged with 6-(dimethylamino)picolinaldehyde (1.5 g, 10 mmol, 1.0 equiv), 2,4,6-trimethylaniline (1.35 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.65 g, 84%). ¹H NMR (500 MHz, CDCl₃) δ 10.23 (s, 1H), 8.21 (d, J=1.7 Hz, 1H), 7.82 (d, J=9.2 Hz, 1H), 7.31 (dd, J=6.9, 2.3 Hz, 1H), 7.04 (s, 2H), 6.61 (d, J=7.1 Hz, 1H), 3.02 (s, 6H), 2.35 (s, 3H), 2.08 (s, 6H). ¹³C NMR (126 MHz, CDCl₃) δ 144.50, 141.27, 133.95, 132.31, 131.40, 129.79, 126.87, 124.53, 115.57, 112.78, 104.31, 42.07, 21.17, 17.66. HRMS calcd for C₁₈H₂₂N₃⁺ (M−Cl⁻) 280.1808, found 280.1810.

### Synthesis of 2-(2,6-Dibenzhydryl-4-methylphenyl)-5-(dimethylamino)imidazo[1,5-a]pyridin-2-ium chloride (3)

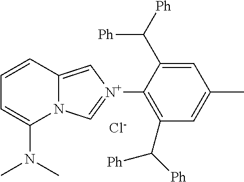 [SMILES: Cc1cc(C(c2ccccc2)c2ccccc2)c(-[n+]2cc3cccc(N(C)C)n3c2)c(C(c2ccccc2)c2ccccc2)c1 ; [Cl-]]

An oven-dried 100 mL round-bottomed flask equipped with a stir bar was charged with 6-(dimethylamino)picolinaldehyde (1.5 g, 10 mmol, 1.0 equiv), 2,6-dibenzhydryl-4-methylaniline (4.40 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (4.83 g, 78%).

¹H NMR (500 MHz, CDCl₃) δ 8.66 (s, 1H), 7.89 (s, 1H), 7.66 (s, 1H), 7.27-7.13 (m, 13H), 7.04 (d, J=7.0 Hz, 4H), 6.88 (d, J=6.7 Hz, 4H), 6.78 (s, 2H), 6.43 (d, J=6.6 Hz, 1H), 5.26 (s, 2H), 2.36 (s, 6H), 2.23 (s, 3H). ¹³C NMR (126 MHz, CDCl₃) δ 142.39, 141.26, 140.40, 140.35, 139.87, 130.40, 130.06, 129.53, 128.56, 127.84, 127.61, 127.57, 125.96, 125.88, 125.00, 124.01, 116.08, 112.32, 102.72, 50.69, 40.29, 20.90. HRMS calcd for C₄₂H₃₈N₃⁺ (M−Cl⁻) 584.3060, found 584.3049.

### Synthesis of 6-(Diethylamino)picolinaldehyde

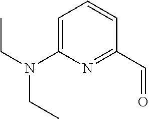

An oven-dried 100 mL round-bottomed flask equipped with a stir bar was charged with 6-bromo-N,N-diethylpyridin-2-amine (4.58 g, 20 mmol, 1.0 equiv) and dry THE (35 mL). n-BuLi (1.6 M in hexane, 15 mL, 1.2 equiv) was added dropwise at −78° C. and the resulting mixture was stirred at −78° C. for 1 hour. After the indicated time, DMF (1.86 mL, 24 mmol, 1.2 equiv) was added dropwise at −78° C. and the reaction mixture was stirred for 1 hour at −78° C. After the indicated time, the reaction mixture was diluted with EtOAc (50 mL), washed with H₂O (1×20 mL) and brine (1×20 mL). The organic layers were combined, dried with Na₂SO₄, filtered, and concentrated. The residue was purified by chromatography on silica gel to afford the title product (3.1 g, 87%). Colorless oil. ¹H NMR (500 MHz, CDCl₃) δ 9.79 (s, 1H), 7.48-7.40 (m, 1H), 7.06 (d, J=7.2 Hz, 1H), 6.56 (d, J=8.6 Hz, 1H), 3.48 (q, J=7.1 Hz, 4H), 1.11 (t, J=7.1 Hz, 6H). ¹³C NMR (126 MHz, CDCl₃) δ 194.85, 157.61, 151.50, 137.56, 110.01, 109.18, 42.48, 12.84. HRMS calcd for C₁₀H₁₅N₂O⁺ (M+H⁺) 179.1179, found 179.1141.

### Synthesis of 5-(Diethylamino)-2-(2,6-diisopropylphenyl)imidazo[1,5-a]pyridin-2-ium chloride (4)

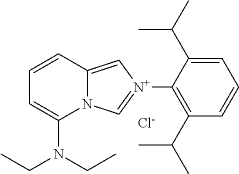 [SMILES: CCN(CC)c1cccc2c[n+](-c3c(C(C)C)cccc3C(C)C)cn12 ; [Cl-]]

An oven-dried 100 mL round-bottomed flask equipped with a stir bar was charged with 6-(diethylamino)picolinaldehyde (1.78 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.93 g, 76%). ¹H NMR (500 MHz, CDCl₃) δ 9.14 (s, 1H), 8.86 (s, 1H), 8.44 (d, J=9.4 Hz, 1H), 7.60 (t, J=7.9 Hz, 1H), 7.47-7.40 (m, 1H), 7.36 (d, J=7.9 Hz, 2H), 6.84 (d, J=7.3 Hz, 1H), 3.25 (q, J=7.1 Hz, 4H), 2.16 (dt, J=13.6, 6.7 Hz, 2H), 1.24 (d, J=6.8 Hz, 6H), 1.18 (d, J=6.9 Hz, 6H), 1.14 (t, J=7.1 Hz, 6H). ¹³C NMR (126 MHz, CDCl₃) δ 145.04, 141.08, 132.84, 132.12, 130.82, 126.71, 124.65, 122.23, 118.91, 116.42, 110.26, 45.89, 28.76, 24.73, 24.08, 11.94. HRMS calcd for C₂₃H₃₂N₃⁺ (M−Cl⁻) 350.2591, found 350.2594.

### Synthesis of 5-(Diethylamino)-2-mesitylimidazo[1,5-a]pyridin-2-ium chloride (5)

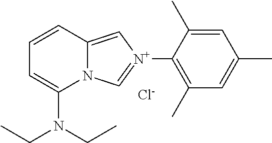 [SMILES: CCN(CC)c1cccc2c[n+](-c3c(C)cc(C)cc3C)cn12 ; [Cl-]]

An oven-dried 100 mL round-bottomed flask equipped with a stir bar was charged with 6-(diethylamino)picolinaldehyde (1.78 g, 10 mmol, 1.0 equiv), 2,4,6-trimethylaniline (1.35 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.75 g, 80%). ¹H NMR (500 MHz, CDCl₃) δ 9.54 (s, 1H), 8.49 (d, J=1.6 Hz, 1H), 8.07 (d, J=9.2 Hz, 1H), 7.35 (t, J=8.2 Hz, 1H), 7.04 (s, 2H), 6.74 (d, J=7.2 Hz, 1H), 3.29 (q, J=7.1 Hz, 4H), 2.35 (s, 3H), 2.06 (s, 6H), 1.13 (t, J=7.1 Hz, 6H). ¹³C NMR (126 MHz, CDCl₃) δ 141.25, 141.03, 133.69, 132.39, 131.19, 129.57, 126.32, 122.79, 116.65, 114.61, 109.21, 45.11, 20.97, 17.41, 11.46. HRMS calcd for C₂₀H₂₆N₃⁺ (M−Cl⁻) 308.2121, found 308.2126.

### Synthesis of 2-(2,6-Dibenzhydryl-4-methylphenyl)-5-(diethylamino)imidazo[1,5-a]pyridin-2-ium chloride (6)

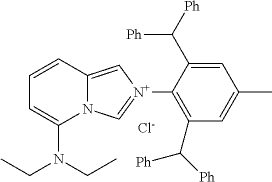 [SMILES: CCN(CC)c1cccc2c[n+](-c3c(C(c4ccccc4)c4ccccc4)cc(C)cc3C(c3ccccc3)c3ccccc3)cn12 ; [Cl-]]

An oven-dried 100 mL round-bottomed flask equipped with a stir bar was charged with 6-(diethylamino)picolinaldehyde (1.78 g, 10 mmol, 1.0 equiv), 2,6-dibenzhydryl-4-methylaniline (4.40 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (4.54 g, 70%). ¹H NMR (500 MHz, CDCl₃) δ 9.12 (d, J=1.3 Hz, 1H), 8.14 (d, J=9.2 Hz, 1H), 7.46 (s, 1H), 7.25-7.11 (m, 13H), 7.02 (d, J=7.3 Hz, 4H), 6.90-6.81 (m, 4H), 6.77 (s, 2H), 6.38 (d, J=7.1 Hz, 1H), 5.25 (s, 2H), 2.52 (q, J=7.0 Hz, 4H), 2.22 (s, 3H), 0.82 (t, J=7.1 Hz, 6H). ¹³C NMR (126 MHz, CDCl₃) δ 142.36, 141.47, 141.32, 140.62, 140.31, 131.99, 131.03, 130.66, 129.69, 128.69, 128.59, 128.53, 127.09, 126.99, 125.77, 123.42, 118.22, 114.99, 107.78, 51.62, 43.57, 21.90, 11.27. HRMS calcd for C₄₄H₄₂N₃⁺ (M−Cl⁻) 612.3373, found 612.3354.

### Synthesis of 2-(2,6-Diisopropylphenyl)-5-(piperidin-1-yl)imidazo[1,5-a]pyridin-2-ium chloride (7)

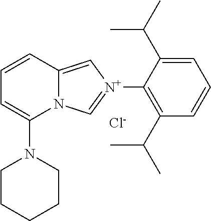 [SMILES: CC(C)c1cccc(C(C)C)c1-[n+]1cc2cccc(N3CCCCC3)n2c1 ; [Cl-]]

An oven-dried 100 mL round-bottomed flask equipped with a stir bar was charged with 6-(piperidin-1-yl)picolinaldehyde (1.9 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.98 g, 75%). ¹H NMR (500 MHz, CDCl₃) δ 8.89 (s, 1H), 8.82 (d, J=1.4 Hz, 1H), 8.34 (d, J=9.2 Hz, 1H), 7.59 (t, J=7.9 Hz, 1H), 7.44-7.27 (m, 3H), 6.77 (d, J=7.3 Hz, 1H), 3.19-3.03 (m, 4H), 2.15 (dd, J=13.6, 6.8 Hz, 2H), 1.88-1.76 (m, 4H), 1.70 (d, J=4.8 Hz, 2H), 1.22 (d, J=6.8 Hz, 6H), 1.18 (d, J=6.8 Hz, 6H). ¹³C NMR (126 MHz, CDCl₃) δ 145.04, 143.28, 132.61, 132.14, 130.79, 127.16, 124.71, 121.43, 118.84, 115.64, 106.72, 51.64, 28.75, 25.61, 24.75, 24.25, 23.83. HRMS caled for C₂₄H₃₂N₃⁺ (M−Cl⁻) 362.2591, found 362.2596.

### Synthesis of 2-Mesityl-5-(piperidin-1-yl)imidazo[1,5-a]pyridin-2-ium chloride (8)

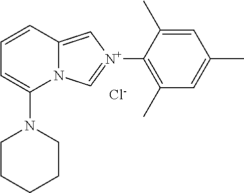 [SMILES: Cc1cc(C)c(-[n+]2cc3cccc(N4CCCCC4)n3c2)c(C)c1 ; [Cl-]]

An oven-dried 100 mL round-bottomed flask equipped with a stir bar was charged with 6-(piperidin-1-yl)picolinaldehyde (1.9 g, 10 mmol, 1.0 equiv), 2,4,6-trimethylaniline (1.35 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.56 g, 72%). ¹H NMR (500 MHz, CDCl₃) δ 9.47 (s, 1H), 8.47 (s, 1H), 8.01 (d, J=9.0 Hz, 1H), 7.31 (t, J=8.0 Hz, 1H), 7.04 (s, 2H), 6.66 (d, J=7.1 Hz, 1H), 3.14 (s, 4H), 2.35 (s, 3H), 2.06 (s, 6H), 1.86 (s, 4H), 1.68 (s, 2H). ¹³C NMR (126 MHz, CDCl₃) δ 143.66, 141.04, 133.80, 132.23, 131.20, 129.54, 126.74, 122.24, 116.56, 114.02, 105.83, 51.45, 25.27, 23.70, 20.99, 17.47. HRMS calcd for C₂₁H₂₆N₃⁺ (M−Cl⁻) 320.2121, found 320.2121.

### Synthesis of 2-(2,6-Dibenzhydryl-4-methylphenyl)-5-(piperidin-1-yl)imidazo[1,5-a]pyridin-2-ium chloride (9)

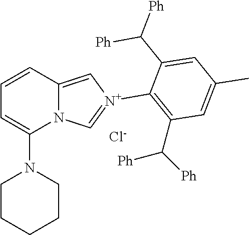 [SMILES: Cc1cc(C(c2ccccc2)c2ccccc2)c(-[n+]2cc3cccc(N4CCCCC4)n3c2)c(C(c2ccccc2)c2ccccc2)c1 ; [Cl-]]

An oven-dried 100 mL round-bottomed flask equipped with a stir bar was charged with 6-(piperidin-1-yl)picolinaldehyde (1.9 g, 10 mmol, 1.0 equiv), 2,6-dibenzhydryl-4-methylaniline (4.40 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (4.49 g, 68%).

¹H NMR (500 MHz, CDCl₃) δ 9.43 (s, 1H), 8.26 (d, J=9.2 Hz, 1H), 7.20 (dq, J=22.4, 7.2 Hz, 7H), 7.14-7.07 (m, 6H), 7.02 (d, J=7.4 Hz, 4H), 6.75 (dd, J=12.8, 7.8 Hz, 7H), 6.38 (d, J=7.2 Hz, 1H), 5.15 (s, 2H), 2.37-2.26 (m, 4H), 2.22 (s, 3H), 1.47 (s, 2H), 1.29 (s, 4H). ¹³C NMR (126 MHz, CDCl₃) δ 142.56, 142.44, 141.59, 141.08, 140.76, 131.95, 130.98, 130.57, 129.75, 128.66, 128.59, 128.32, 127.31, 127.02, 126.20, 122.91, 118.10, 115.29, 105.46, 51.58, 50.85, 25.40, 23.63, 21.90. HRMS calcd for C₄₅H₄₂N₃⁺ (M−Cl⁻) 624.3373, found 624.3353.

### Synthesis of 2-(2,6-Diisopropylphenyl)-5-morpholinoimidazo[1,5-a]pyridin-2-ium chloride (10)

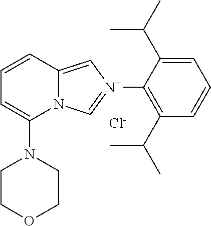 [SMILES: CC(C)c1cccc(C(C)C)c1-[n+]1cc2cccc(N3CCOCC3)n2c1 ; [Cl-]]

An oven-dried 100 mL round-bottomed flask equipped with a stir bar was charged with 6-morpholinopicolinaldehyde (1.92 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.8 g, 70%). ¹H NMR (500 MHz, CDCl₃) δ 10.28 (d, J=0.8 Hz, 1H), 8.29 (d, J=1.6 Hz, 1H), 7.99 (d, J=9.2 Hz, 1H), 7.54 (t, J=7.8 Hz, 1H), 7.43-7.34 (m, 1H), 7.31 (d, J=7.9 Hz, 2H), 6.80 (d, J=7.2 Hz, 1H), 4.05-3.94 (m, 4H), 3.25-3.14 (m, 4H), 2.11 (dt, J=13.6, 6.8 Hz, 2H), 1.21 (d, J=6.8 Hz, 6H), 1.14 (d, J=6.8 Hz, 6H). ¹³C NMR (126 MHz, CDCl₃) δ 144.79, 142.87, 131.94, 131.88, 130.82, 127.11, 124.42, 117.24, 114.59, 106.33, 66.23, 50.51, 28.68, 24.43, 24.31. HRMS calcd for C₂₃H₃₀N₃O⁺ (M−Cl⁻) 364.2383, found 364.2386.

### Synthesis of 2-Mesityl-5-morpholinoimidazo[1,5-a]pyridin-2-ium chloride (11)

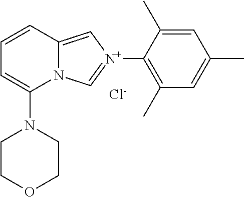 [SMILES: Cc1cc(C)c(-[n+]2cc3cccc(N4CCOCC4)n3c2)c(C)c1 ; [Cl-]]

An oven-dried 100 mL round-bottomed flask equipped with a stir bar was charged with 6-morpholinopicolinaldehyde (1.92 g, 10 mmol, 1.0 equiv), 2,4,6-trimethylaniline (1.35 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.79 g, 78%). ¹H NMR (500 MHz, CDCl₃) δ 10.70 (s, 1H), 7.94 (s, 1H), 7.72 (d, J=9.1 Hz, 1H), 7.25 (d, J=7.9 Hz, 1H), 6.96 (s, 2H), 6.60 (d, J=7.1 Hz, 1H), 4.01 (s, 4H), 3.18 (s, 4H), 2.27 (s, 3H), 2.02 (s, 6H). ¹³C NMR (126 MHz, CDCl₃) δ 143.62, 141.16, 134.02, 131.95, 131.45, 129.74, 126.67, 125.53, 115.41, 113.78, 105.31, 66.37, 50.68, 21.19, 17.80. HRMS calcd for C₂₀H₂₄N₃O⁺ (M−Cl⁻) 322.1914, found 322.1917.

### Synthesis of 2-(2,6-Dibenzhydryl-4-methylphenyl)-5-morpholinoimidazo[1,5-a]pyridin-2-ium chloride (12)

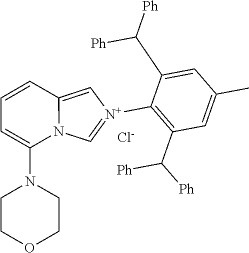 [SMILES: Cc1cc(C(c2ccccc2)c2ccccc2)c(-[n+]2cc3cccc(N4CCOCC4)n3c2)c(C(c2ccccc2)c2ccccc2)c1 ; [Cl-]]

An oven-dried 100 mL round-bottomed flask equipped with a stir bar was charged with 6-morpholinopicolinaldehyde (1.92 g, 10 mmol, 1.0 equiv), 2,6-dibenzhydryl-4-methylaniline (4.40 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (4.30 g, 65%).

¹H NMR (500 MHz, CDCl₃) δ 8.96 (d, J=1.4 Hz, 1H), 8.15 (d, J=9.2 Hz, 1H), 7.55 (s, 1H), 7.30-7.10 (m, 13H), 7.02 (d, J=7.2 Hz, 4H), 6.88 (d, J=6.4 Hz, 4H), 6.77 (s, 2H), 6.45 (d, J=7.1 Hz, 1H), 5.23 (s, 2H), 3.54 (d, J=4.3 Hz, 4H), 2.45 (s, 4H), 2.24 (s, 3H). ¹³C NMR (126 MHz, CDCl₃) δ 142.67, 141.67, 141.53, 141.24, 140.77, 131.55, 130.95, 130.60, 129.71, 128.72, 128.63, 128.59, 127.07, 127.06, 125.90, 123.66, 118.22, 115.83, 105.68, 66.14, 51.52, 49.76, 21.93. HRMS calcd for C₄₄H₄₀N₃O⁺ (M−Cl⁻) 626.3166, found 626.3169.

### Synthesis of 6-(3,5-Diphenyl-H-pyrazol-1-yl)picolinaldehyde

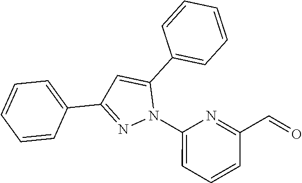 [SMILES: O=Cc1cccc(-n2nc(-c3ccccc3)cc2-c2ccccc2)n1]

An oven-dried 100 mL round-bottomed flask equipped with a stir bar was charged with 2-bromo-6-(3,5-diphenyl-1H-pyrazol-1-yl)pyridine (7.52 g, 20 mmol, 1.0 equiv) and dry THE (35 mL). n-BuLi (1.6 M in hexane, 15 mL, 1.2 equiv) was added dropwise at −78° C. and the resulting mixture was stirred at −78° C. for 1 hour. After the indicated time, DMF (1.86 mL, 24 mmol, 1.2 equiv) was added dropwise at −78° C. and the reaction was stirred for 1 hour at −78° C. After the indicate time, the reaction mixture was diluted with EtOAc (50 mL), washed with H₂O (1×20 mL) and brine (1×20 mL). The organic layers were combined, dried, filtered, and concentrated. The residue was purified by chromatography on silica gel to afford the title product (2.6 g, 40%). ¹H NMR (500 MHz, CDCl₃) δ 9.54 (s, 1H), 8.05 (d, J=8.1 Hz, 1H), 7.96 (t, J=8.0 Hz, 3H), 7.83 (d, J=7.5 Hz, 1H), 7.48 (t, J=7.5 Hz, 2H), 7.43-7.34 (m, 6H), 6.87 (s, 1H). ¹³C NMR (126 MHz, CDCl₃) δ 192.57, 153.10, 152.85, 150.91, 145.72, 139.34, 132.49, 131.57, 129.11, 128.76, 128.57, 128.47, 128.07, 126.06, 121.84, 119.15, 107.49. HRMS caled for C₂₁H₁₅N₃O⁺ (M+Na⁺) 348.1107, found 348.1113.

### Synthesis of 2-(2,6-Diisopropylphenyl)-5-(3,5-diphenyl-1H-pyrazol-1-yl)imidazo[1,5-a]pyridin-2-ium chloride (13)

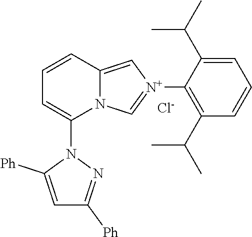 [SMILES: CC(C)c1cccc(C(C)C)c1-[n+]1cc2cccc(-n3nc(-c4ccccc4)cc3-c3ccccc3)n2c1 ; [Cl-]]

An oven-dried 100 mL round-bottomed flask equipped with a stir bar was charged with 6-(3,5-diphenyl-1H-pyrazol-1-yl)picolinaldehyde (3.25 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (3.3 g, 62%). ¹H NMR (500 MHz, CDCl₃) δ 9.56 (s, 1H), 9.15 (d, J=9.1 Hz, 1H), 9.03 (s, 1H), 7.92-7.78 (m, 2H), 7.60 (t, J=7.9 Hz, 1H), 7.54-7.41 (m, 6H), 7.39-7.29 (m, 5H), 7.05 (s, 1H), 6.80 (d, J=7.1 Hz, 1H), 2.27 (dt, J=13.5, 6.8 Hz, 2H), 1.30 (d, J=6.8 Hz, 6H), 1.19 (d, J=6.8 Hz, 6H). ¹³C NMR (126 MHz, CDCl₃) δ 155.48, 147.86, 145.06, 133.11, 132.30, 130.90, 130.64, 130.31, 129.66, 129.54, 129.02, 128.86, 128.01, 127.63, 125.93, 124.79, 124.74, 123.01, 122.41, 121.00, 116.10, 106.69, 28.80, 24.67, 24.20. HRMS calcd for C₃₄H₃₃N₄⁺ (M−Cl⁻) 497.2700, found 497.2699.

### Synthesis of 5-(9H-Carbazol-9-yl)-2-(2,6-diisopropylphenyl)imidazo[1,5-a]pyridin-2-ium chloride (14)

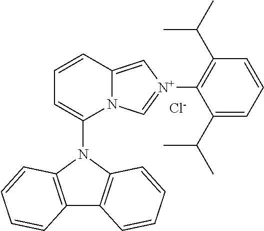 [SMILES: CC(C)c1cccc(C(C)C)c1-[n+]1cc2cccc(-n3c4ccccc4c4ccccc43)n2c1 ; [Cl-]]

An oven-dried 100 mL round-bottomed flask equipped with a stir bar was charged with 6-(9H-carbazol-9-yl)picolinaldehyde (2.72 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (3.22 g, 67%). ¹H NMR (500 MHz, CDCl₃) δ 9.55 (s, 1H), 9.26 (d, J=9.3 Hz, 1H), 8.15 (d, J=7.6 Hz, 2H), 7.82 (s, 1H), 7.73-7.64 (m, 1H), 7.58 (d, J=7.0 Hz, 1H), 7.45 (tt, J=23.8, 7.6 Hz, 5H), 7.24 (d, J=7.9 Hz, 2H), 7.14 (d, J=8.0 Hz, 2H), 2.09 (dt, J=13.5, 6.8 Hz, 2H), 1.23 (d, J=6.8 Hz, 6H), 0.94 (d, J=6.8 Hz, 6H). ¹³C NMR (126 MHz, CDCl₃) δ 144.88, 138.75, 133.30, 132.29, 130.23, 127.54, 127.49, 125.95, 124.66, 124.60, 123.03, 122.87, 121.93, 121.44, 121.15, 119.68, 109.47, 28.76, 24.43, 24.33. HRMS calcd for C₃₁H₃₀N₃⁺ (M−Cl⁻) 444.2434, found 444.2433.

### Synthesis of 2-(2,6-Diisopropylphenyl)-5-methoxyimidazo[1,5-a]pyridin-2-ium chloride (15)

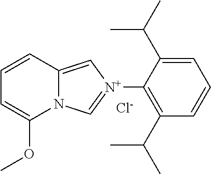 [SMILES: COc1cccc2c[n+](-c3c(C(C)C)cccc3C(C)C)cn12 ; [Cl-]]

An oven-dried 50 mL round-bottomed flask equipped with a stir bar was charged with 6-methoxypicolinaldehyde (685 mg, 5 mmol, 1.0 equiv), 2,6-diisopropylaniline (885 mg, 5 mmol, 1.0 equiv), paraformaldehyde (225 mg, 7.5 mmol, 1.5 equiv) and EtOH (12 mL). HCl (4.0 M in dioxane, 2.5 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (1.3 g, 75%). ¹H NMR (500 MHz, Chloroform-d) δ 9.52 (d, J=2.0 Hz, 1H), 8.30 (d, J=1.9 Hz, 1H), 7.88 (d, J=9.2 Hz, 1H), 7.56 (t, J=7.8 Hz, 1H), 7.44 (dd, J=9.2, 7.6 Hz, 1H), 7.32 (d, J=7.9 Hz, 2H), 6.71 (d, J=7.5 Hz, 1H), 4.31 (s, 3H), 2.12 (p, J=6.8 Hz, 2H), 1.16 (dd, J=12.2, 6.8 Hz, 12H). ¹³C NMR (126 MHz, CDCl₃) δ 146.72, 145.07, 132.16, 132.14, 130.59, 128.43, 124.63, 122.12, 116.55, 111.19, 93.99, 58.36, 28.68, 24.49, 24.46. HRMS caled for C₂₀H₂₅N₂O⁺ (M−Cl⁻) 309.1961, found 309.1973.

### Example 2: Synthesis of Imidazo[1,5-a]pyridine NHC Ligand-Metal Complexes

### Synthesis of 2-(2,6-Diisopropylphenyl)-5-(dimethylamino)imidazo[1,5-a]pyridin-3-ylidene gold(I) chloride (16)

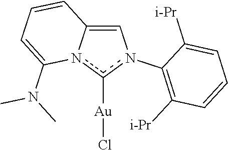

An oven-dried flask equipped with a stir bar was charged with the corresponding NHC·HCl salt (1) (2-(2,6-Diisopropylphenyl)-5-(dimethylamino)imidazo[1,5-a]pyridin-2-ium chloride) (107 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (150 mg, 90%). ¹H NMR (500 MHz, CDCl₃) δ 7.49 (t, J=7.8 Hz, 1H), 7.31-7.22 (m, 3H), 7.17 (d, J=9.0 Hz, 1H), 6.98 (dd, J=8.9, 7.2 Hz, 1H), 6.27 (d, J=6.9 Hz, 1H), 2.94 (s, 6H), 2.21 (dt, J=13.8, 6.9 Hz, 2H), 1.31 (d, J=6.8 Hz, 6H), 1.10 (d, J=6.8 Hz, 6H). ¹³C NMR (126 MHz, CDCl₃) δ 164.49, 148.63, 145.20, 135.78, 132.55, 130.57, 124.27, 124.12, 113.75, 112.50, 102.48, 44.28, 28.45, 24.50, 24.32. HRMS calcd for C₂₁H₂₇AuClN₃Na⁺ (M++Na) 576.1451, found 576.1456.

### Synthesis of 5-(Dimethylamino)-2-mesitylimidazo[1,5-a]pyridin-3-ylidene gold(I) chloride (17)

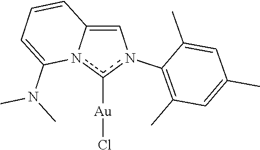

An oven-dried flask equipped with a stir bar was charge with the corresponding NHC·HCl salt (2) (95 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (111 mg, 72%). ¹H NMR (500 MHz, CDCl₃) δ 7.22 (s, 1H), 7.16 (d, J=9.0 Hz, 1H), 7.01-6.92 (m, 3H), 6.26 (d, J=7.0 Hz, 1H), 2.95 (s, 6H), 2.36 (s, 3H), 2.00 (s, 6H). ¹³C NMR (126 MHz, CDCl₃) δ 163.47, 148.71, 139.62, 136.41, 134.27, 132.87, 129.38, 123.97, 112.62, 112.44, 102.41, 44.32, 21.19, 17.83. HRMS calcd for C₁₈H₂₁AuClN₃Na⁺ (M⁺+Na) 534.0982, found 534.0986.

### Synthesis of 2-(2,6-Dibenzhydryl-4-methylphenyl)-5-(dimethylamino)imidazo[1,5-a]pyridin-3-ylidene gold(I) chloride (18)

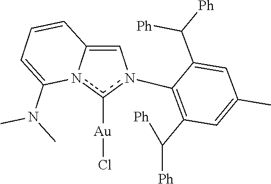

An oven-dried flask equipped with a stir bar was charged with the corresponding NHC·HCl salt (3) (186 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (198 mg, 81%). ¹H NMR (500 MHz, CDCl₃) δ 7.24 (d, J=7.6 Hz, 4H), 7.20-7.08 (m, 13H), 6.79 (td, J=7.3, 2.9 Hz, 4H), 6.75 (s, 2H), 6.63 (d, J=9.0 Hz, 1H), 6.14 (d, J=6.8 Hz, 1H), 5.86 (s, 1H), 5.32 (s, 2H), 2.82 (s, 6H), 2.24 (s, 3H). ¹³C NMR (126 MHz, CDCl₃) δ 163.95, 148.20, 142.50, 142.16, 141.09, 139.67, 135.99, 131.37, 130.16, 129.61, 129.26, 128.46, 128.32, 126.58, 126.53, 123.30, 115.08, 112.44, 101.94, 51.45, 44.08, 21.88. HRMS caled for C₄₂H₃₇AuClN₃Na⁺ (M⁺+Na) 838.2234, found 838.2221.

### Synthesis of 5-(Diethylamino)-2-(2,6-diisopropylphenyl)imidazo[1,5-a]pyridin-3-ylidene gold(I) chloride (19)

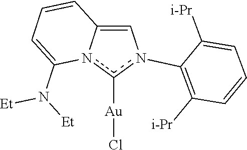

An oven-dried flask equipped with a stir bar was charged with the corresponding NHC·HCl salt (4) (116 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (162 mg, 93%). ¹H NMR (500 MHz, CDCl₃) δ 7.49 (t, J=7.8 Hz, 1H), 7.37-7.14 (m, 4H), 6.98 (dd, J=8.9, 7.1 Hz, 1H), 6.33 (d, J=6.9 Hz, 1H), 3.32 (dd, J=14.1, 7.0 Hz, 4H), 2.21 (dt, J=13.6, 6.7 Hz, 2H), 1.31 (d, J=6.8 Hz, 6H), 1.20 (t, J=7.1 Hz, 6H), 1.12 (d, J=6.9 Hz, 6H). ¹³C NMR (126 MHz, CDCl₃) δ 164.66, 145.58, 145.18, 135.90, 132.47, 130.49, 124.08, 123.91, 113.78, 113.17, 107.17, 46.44, 28.44, 24.46, 24.35, 11.07. HRMS calcd for C₂₃H₃₁AuClN₃Na⁺ (M⁺+Na) 604.1764, found 604.1770.

### Synthesis of 5-(Diethylamino)-2-mesitylimidazo[1,5-a]pyridin-3-ylidene gold(I) chloride (20)

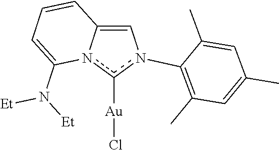

An oven-dried flask equipped with a stir bar was charged with the corresponding NHC·HCl salt (5) (103 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (110 mg, 68%). ¹H NMR (500 MHz, CDCl₃) δ 7.20 (d, J=8.8 Hz, 2H), 6.99-6.90 (m, 3H), 6.34-6.27 (m, 1H), 3.31 (q, J=7.0 Hz, 4H), 2.33 (s, 3H), 1.98 (s, 6H), 1.18 (t, J=7.1 Hz, 6H). ¹³C NMR (126 MHz, CDCl₃) δ 163.40, 145.45, 139.47, 136.53, 134.24, 132.83, 129.31, 123.64, 113.37, 112.54, 107.13, 46.48, 21.18, 17.86, 11.08. HRMS calcd for C₂₀H₂₅AuClN₃Na⁺ (M⁺+Na) 562.1295, found 562.1304.

### Synthesis of 2-(2,6-Dibenzhydryl-4-methylphenyl)-5-(diethylamino)imidazo[1,5-a]pyridin-3-ylidene gold(I) chloride (21)

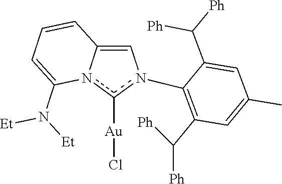

An oven-dried flask equipped with a stir bar was charged with the corresponding NHC·HCl salt (6) (194 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (164 mg, 65%). ¹H NMR (500 MHz, CDCl₃) δ 7.18 (dd, J=9.5, 5.4 Hz, 4H), 7.13-7.02 (m, 12H), 6.75-6.67 (m, 5H), 6.65 (s, 2H), 6.60 (d, J=9.0 Hz, 1H), 6.12 (d, J=6.7 Hz, 1H), 5.81 (s, 1H), 5.29 (s, 2H), 3.11 (dd, J=7.0, 3.6 Hz, 4H), 2.15 (s, 3H), 1.08 (t, J=7.1 Hz, 6H). ¹³C NMR (126 MHz, CDCl₃) δ 164.04, 145.21, 142.51, 142.09, 141.04, 139.53, 136.09, 131.28, 130.11, 129.60, 129.25, 128.46, 128.32, 126.57, 126.51, 122.94, 114.95, 113.18, 106.70, 51.53, 46.37, 21.86, 11.16. HRMS calcd for C₄₄H₄₁AuClN₃Na⁺ (M⁺+Na) 866.2547, found 866.2533.

### Synthesis of 2-(2,6-Diisopropylphenyl)-5-(piperidin-1-yl)imidazo[1,5-a]pyridin-3-ylidene gold(I) chloride (22)

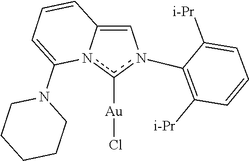

An oven-dried flask equipped with a stir bar was charged with the corresponding NHC·HCl salt (7) (119 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (200 mL) and filtered. The solution was collected and concentrated. The title product was obtained by trituration from hexanes as a white solid (151 mg, 85%). ¹H NMR (500 MHz, CDCl₃) δ 7.49 (t, J=7.8 Hz, 1H), 7.25 (d, J=5.8 Hz, 3H), 7.17 (d, J=9.0 Hz, 1H), 6.97 (dd, J=9.0, 7.1 Hz, 1H), 6.30 (d, J=7.0 Hz, 1H), 3.55-3.39 (m, 2H), 2.65 (t, J=10.8 Hz, 2H), 2.42 (td, J=12.8, 3.7 Hz, 2H), 2.20 (dq, J=13.7, 6.8 Hz, 2H), 1.96 (d, J=13.0 Hz, 1H), 1.70 (d, J=13.3 Hz, 2H), 1.31 (d, J=6.8 Hz, 7H), 1.10 (d, J=6.9 Hz, 6H). ¹³C NMR (126 MHz, CDCl₃) δ 164.34, 148.90, 145.18, 135.86, 132.46, 130.54, 124.32, 124.11, 113.76, 112.84, 103.06, 53.62, 28.46, 25.26, 24.51, 24.30, 24.20. HRMS calcd for C₂₄H₃₁AuClN₃Na⁺ (M⁺+Na) 616.1764, found 616.1760.

### Synthesis of 2-Mesityl-5-(piperidin-1-yl)imidazo[1,5-a]pyridin-3-ylidene gold(I) chloride (23)

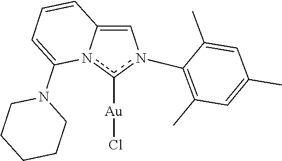

An oven-dried flask equipped with a stir bar was charged with the corresponding NHC·HCl salt (8) (107 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (200 mL) and filtered. The solution was collected and concentrated. The title product was obtained by trituration from hexanes as a white solid (104 mg, 63%). ¹H NMR (500 MHz, CDCl₃) δ 7.22 (s, 1H), 7.18 (d, J=8.8 Hz, 1H), 7.01-6.92 (m, 3H), 6.30 (d, J=6.6 Hz, 1H), 3.49 (d, J=10.9 Hz, 2H), 2.72-2.61 (m, 2H), 2.44 (ddt, J=16.1, 12.6, 6.4 Hz, 2H), 2.34 (s, 3H), 1.98 (d, J=15.0 Hz, 7H), 1.72 (d, J=13.3 Hz, 2H), 1.44-1.31 (m, 1H). ¹³C NMR (126 MHz, CDCl₃) δ 163.10, 148.92, 139.54, 136.51, 134.26, 132.82, 129.34, 124.02, 113.07, 112.52, 103.17, 53.69, 25.32, 24.20, 21.18, 17.87. HRMS calcd for C₂₁H₂₅AuClN₃Na⁺ (M⁺+Na) 574.1295, found 574.1297.

### Synthesis of 2-(2,6-Dibenzhydryl-4-methylphenyl)-5-(piperidin-1-yl)imidazo[1,5-a]pyridin-3-ylidene gold(I) chloride (24)

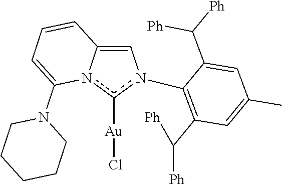

An oven-dried flask equipped with a stir bar was charged with the corresponding NHC·HCl salt (9) (198 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (200 mL) and filtered. The solution was collected and concentrated. The title product was obtained by trituration from hexanes as a white solid (154 mg, 60%). ¹H NMR (500 MHz, CDCl₃) δ 7.24 (d, J=7.6 Hz, 4H), 7.16 (ddd, J=21.6, 15.7, 7.3 Hz, 12H), 6.83-6.76 (m, 5H), 6.75 (s, 2H), 6.68 (d, J=9.0 Hz, 1H), 6.15 (d, J=6.9 Hz, 1H), 5.98 (s, 1H), 5.29 (s, 2H), 3.23 (d, J=10.7 Hz, 2H), 2.50 (t, J=10.8 Hz, 2H), 2.30 (dd, J=25.7, 12.8 Hz, 2H), 2.21 (d, J=15.1 Hz, 3H), 1.95 (d, J=13.0 Hz, 1H), 1.62 (d, J=13.1 Hz, 2H), 1.37-1.26 (m, 1H). ¹³C NMR (126 MHz, CDCl₃) δ 163.96, 148.42, 142.68, 142.01, 141.05, 139.62, 136.07, 131.42, 130.15, 129.63, 129.35, 128.45, 128.32, 126.53, 126.51, 123.48, 114.83, 112.77, 102.49, 53.32, 51.40, 25.36, 24.19, 21.87. HRMS caled for C₄₅H₄₁AuClN₃Na⁺ (M⁺+Na) 878.2547, found 878.2536.

### Synthesis of 2-(2,6-Diisopropylphenyl)-5-methoxyimidazo[1,5-a]pyridin-3-ylidene gold(I) chloride (25)

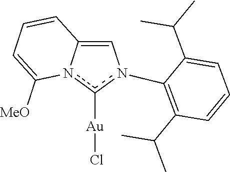

An oven-dried flask equipped with a stir bar was charged with the corresponding NHC·HCl salt (10) (103 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by chromatography on silica gel to afford the title product as a white solid (57 mg, 35%). ¹H NMR (500 MHz, CDCl₃) δ 7.52 (t, J=7.7 Hz, 1H), 7.30 (dd, J=7.9, 2.1 Hz, 2H), 7.14-7.09 (m, 1H), 7.05 (t, J=7.9 Hz, 1H), 5.98 (d, J=7.1 Hz, 1H), 4.15 (d, J=2.1 Hz, 3H), 2.24 (p, J=6.9 Hz, 2H), 1.32 (dd, J=6.9, 2.0 Hz, 6H), 1.12 (dd, J=7.0, 2.2 Hz, 6H). ¹³C NMR (126 MHz, CDCl₃) δ 162.07, 150.39, 145.23, 135.47, 132.13, 130.64, 124.83, 124.16, 113.12, 109.51, 89.06, 56.68, 28.39, 24.53, 24.37. HRMS calcd for C₂₀H₂₄AuClN₂ONa⁺ (M⁺+Na) 563.1135, found 563.1170.

### Synthesis of cinnamyl[2-(2,6-Diisopropylphenyl)-5-(dimethylamino)imidazo[1,5-a]pyridine-3-ylidene]chloropalladium(II) (26)

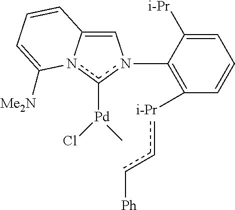

An oven-dried flask equipped with a stir bar was charged with the corresponding NHC·HCl salt (1) (157 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (181 mg, 78%). ¹H NMR (500 MHz, CDCl₃) δ 7.59 (t, J=7.8 Hz, 1H), 7.43-7.28 (m, 10H), 7.21-7.15 (m, 1H), 5.90 (dd, J=22.6, 9.0 Hz, 1H), 4.99 (d, J=13.3 Hz, 1H), 3.03 (s, 6H), 2.54 (d, J=9.3 Hz, 2H), 2.29 (dd, J=13.3, 6.6 Hz, 2H), 1.22 (d, J=6.8 Hz, 6H), 1.15 (d, J=6.8 Hz, 6H). ¹³C NMR (151 MHz, CDCl₃) δ 165.54, 148.39, 145.29, 136.84, 135.50, 131.31, 130.76, 129.28, 127.56, 126.32, 126.08, 124.01, 118.74, 115.87, 114.00, 100.73, 77.00, 49.98, 38.16, 28.39, 24.90, 23.67. HRMS calcd for C₃₀H₃₆N₃Pd⁺ (M−Cl⁻) 544.1939, found 544.1937.

### Synthesis of cinnamyl[5-(Dimethylamino)-2-mesitylimidazo[1,5-a]pyridin-3-ylidene]chloropalladium(I) (27)

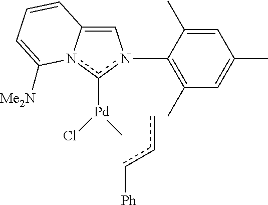

An oven-dried flask equipped with a stir bar was charged with the corresponding NHC·HCl salt (2) (139 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (97 mg, 45%). ¹H NMR (500 MHz, CDCl₃) δ 7.21 (ddd, J=21.9, 14.8, 7.6 Hz, 5H), 7.14-7.09 (m, 2H), 7.00 (s, 2H), 6.94 (dd, J=9.0, 7.1 Hz, 1H), 6.75 (d, J=6.9 Hz, 1H), 5.95 (dt, J=14.1, 9.0 Hz, 1H), 5.29 (d, J=14.1 Hz, 1H), 2.97 (s, 6H), 2.58 (d, J=9.0 Hz, 2H), 2.33 (s, 3H), 2.00 (s, 6H). ¹³C NMR (126 MHz, CDCl₃) δ 163.87, 149.00, 139.89, 138.53, 135.86, 134.77, 134.15, 131.85, 129.24, 128.87, 128.70, 128.25, 127.97, 126.47, 126.03, 124.76, 115.71, 112.89, 105.09, 49.22, 21.24, 17.89. HRMS calcd for C₂₇H₃₀N₃Pd⁺ (M−Cl⁻) 502.1469, found 502.1468.

### Synthesis of cinnamyl[2-(2,6-Dibenzhydryl-4-methylphenyl)-5-(dimethylamino)imidazo[1,5-a]pyridin-3-ylidene]chloropalladium(II) (28)

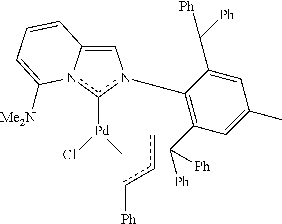

An oven-dried flask equipped with a stir bar was charged with the corresponding NHC·HCl salt (3) (273 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (253 mg, 75%). ¹H NMR (500 MHz, CDCl₃) δ 7.59 (d, J=7.0 Hz, 1H), 7.30-7.25 (m, 3H), 7.19 (dd, J=7.0, 4.0 Hz, 8H), 7.15-7.09 (m, 6H), 7.05 (dd, J=9.1, 7.1 Hz, 1H), 6.95-6.88 (m, 7H), 6.78-6.71 (m, 4H), 6.28 (s, 1H), 5.37 (dt, J=13.1, 9.2 Hz, 1H), 4.98 (s, 2H), 4.50 (d, J=13.3 Hz, 1H), 2.89 (s, 6H), 2.28 (s, 3H), 2.22 (d, J=9.1 Hz, 2H). ¹³C NMR (126 MHz, CDCl₃) δ 166.53, 148.06, 142.66, 141.29, 141.05, 140.06, 136.11, 135.42, 129.81, 129.63, 129.58, 128.76, 128.66, 128.53, 128.24, 126.90, 126.80, 126.59, 126.11, 115.83, 114.87, 114.33, 107.81, 95.36, 77.10, 51.87, 50.45, 43.18, 21.97. HRMS calcd for C₅₁H₄₆N₃Pd⁺ (M−Cl⁻) 806.2721, found 806.2713.

### Synthesis of cinnamyl[5-(Diethylamino)-2-(2,6-diisopropylphenyl)imidazo[1,5-a]pyridin-3-ylidene]chloropalladium(II) (29)

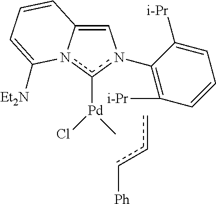

An oven-dried flask equipped with a stir bar was charged with the corresponding NHC·HCl salt (4) (170 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (148 mg, 61%). ¹H NMR (500 MHz, CDCl₃) δ 7.49 (t, J=7.8 Hz, 1H), 7.31-7.22 (m, 3H), 7.17 (d, J=9.0 Hz, 1H), 6.98 (dd, J=8.9, 7.2 Hz, 1H), 6.27 (d, J=6.9 Hz, 1H), 2.94 (s, 6H), 2.21 (dt, J=13.8, 6.9 Hz, 2H), 1.31 (d, J=6.8 Hz, 6H), 1.10 (d, J=6.8 Hz, 6H). ¹³C NMR (126 MHz, CDCl₃) δ 167.18, 145.13, 144.53, 141.70, 135.83, 135.70, 134.79, 131.50, 130.98, 129.77, 128.69, 127.20, 126.16, 124.08, 122.82, 116.21, 113.18, 109.23, 72.21, 66.27, 57.74, 28.51, 24.34, 23.88, 13.32. HRMS calcd for C₃₂H₄₀N₃Pd⁺ (M−Cl⁻) 572.2252, found 572.2248.

### Synthesis of cinnamyl[5-(Diethylamino)-2-mesitylimidazo[1,5-a]pyridin-3-ylidene]chloropalladium(II) (30)

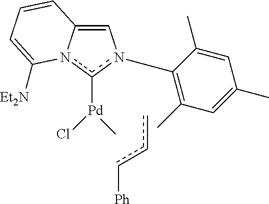

An oven-dried flask equipped with a stir bar was charged with the corresponding NHC·HCl salt (5) (151 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (79 mg, 35%). ¹H NMR (500 MHz, CDCl₃) δ 7.49 (t, J=7.8 Hz, 1H), 7.31-7.22 (m, 3H), 7.17 (d, J=9.0 Hz, 1H), 6.98 (dd, J=8.9, 7.2 Hz, 1H), 6.27 (d, J=6.9 Hz, 1H), 2.94 (s, 6H), 2.21 (dt, J=13.8, 6.9 Hz, 2H), 1.31 (d, J=6.8 Hz, 6H), 1.10 (d, J=6.8 Hz, 6H). ¹³C NMR (126 MHz, CDCl₃) δ 164.61, 144.79, 140.13, 137.33, 135.77, 134.42, 131.56, 129.34, 129.26, 127.49, 125.86, 125.71, 116.38, 112.39, 107.38, 57.15, 21.25, 17.59, 13.17. HRMS calcd for C₂₉H₃₄N₃Pd⁺ (M−Cl⁻) 530.1782, found 530.1779.

### Synthesis of cinnamyl[2-(2,6-Dibenzhydryl-4-methylphenyl)-5-(diethylamino)imidazo[1,5-a]pyridin-3-ylidene]chloropalladium(II) (31)

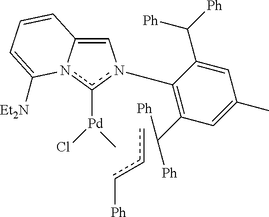

An oven-dried flask equipped with a stir bar was charged with the corresponding NHC·HCl salt (6) (285 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (237 mg, 68%). ¹H NMR (500 MHz, CDCl₃) δ 7.94 (d, J=7.0 Hz, 1H), 7.29 (dd, J=4.9, 1.4 Hz, 2H), 7.21 (dd, J=5.0, 2.9 Hz, 5H), 7.16 (dd, J=7.9, 3.4 Hz, 4H), 7.11 (dd, J=5.8, 3.6 Hz, 6H), 6.97 (t, J=8.4 Hz, 2H), 6.94-6.87 (m, 6H), 6.72-6.64 (m, 4H), 6.27 (s, 1H), 5.36-5.28 (m, 1H), 4.92 (s, 2H), 4.40 (d, J=12.9 Hz, 1H), 3.61 (s, 2H), 2.59 (s, 2H), 2.29 (s, 5H), 1.00 (s, 6H). ¹³C NMR (126 MHz, CDCl₃) δ 166.64, 144.35, 142.68, 141.09, 140.70, 140.14, 135.80, 135.14, 130.80, 129.99, 129.74, 129.35, 128.81, 128.62, 128.60, 127.03, 126.93, 126.80, 126.14, 115.88, 114.08, 112.52, 109.30, 90.41, 57.71, 51.80, 47.36, 21.98, 13.84. HRMS caled for C₅₃H₅₀N₃Pd⁺ (M−Cl⁻) 834.3034, found 834.3020.

### Synthesis of cinnamyl[2-(2,6-Diisopropylphenyl)-5-(piperidin-1-yl)imidazo[1,5-a]pyridin-3-ylidene]chloropalladium(II) (32)

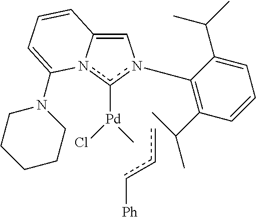

An oven-dried flask equipped with a stir bar was charged with the corresponding NHC·HCl salt (7) (175 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (176 mg, 71%). ¹H NMR (500 MHz, CDCl₃) δ 7.42 (t, J=7.8 Hz, 1H), 7.25 (dd, J=17.4, 7.5 Hz, 4H), 7.21-7.08 (m, 4H), 7.02 (d, J=9.0 Hz, 1H), 6.77 (dd, J=9.0, 7.1 Hz, 1H), 6.23 (d, J=6.9 Hz, 1H), 5.26 (dt, J=12.7, 9.2 Hz, 1H), 4.57-4.48 (m, 1H), 3.34 (s, 2H), 2.50 (dd, J=21.4, 14.7 Hz, 4H), 2.30 (d, J=7.6 Hz, 2H), 1.69 (s, 6H), 1.21 (d, J=6.8 Hz, 6H), 0.99 (d, J=6.8 Hz, 6H). ¹³C NMR (126 MHz, CDCl₃) δ 171.48, 149.74, 146.03, 138.10, 137.15, 133.36, 130.02, 128.39, 127.01, 126.63, 124.19, 123.46, 114.90, 113.41, 107.73, 103.38, 88.43, 77.10, 53.74, 44.45, 28.18, 26.14, 25.87, 23.85, 23.02. HRMS calcd for C₃₃H₄₀N₃Pd⁺ (M−Cl⁻) 584.2252, found 584.2247.

### Synthesis of cinnamyl[2-Mesityl-5-(piperidin-1-yl)imidazo[1,5-a]pyridin-3-ylidene]chloropalladium(II) (33)

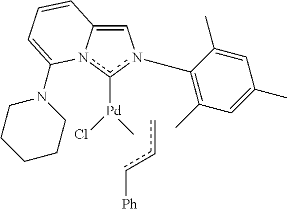

An oven-dried flask equipped with a stir bar was charged with the corresponding NHC·HCl salt (8) (157 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (120 mg, 52%). ¹H NMR (500 MHz, CDCl₃) δ 7.24 (d, J=7.4 Hz, 2H), 7.16 (dd, J=16.2, 6.6 Hz, 3H), 7.10 (t, J=7.2 Hz, 1H), 7.00 (d, J=9.0 Hz, 1H), 6.93 (s, 2H), 6.73 (dd, J=8.9, 7.0 Hz, 1H), 6.14 (d, J=6.9 Hz, 1H), 5.27 (dt, J=12.3, 9.3 Hz, 1H), 4.48 (d, J=12.5 Hz, 1H), 3.41 (s, 2H), 2.54 (s, 4H), 2.31 (s, 3H), 2.02 (s, 6H), 1.89-1.59 (m, 6H). ¹³C NMR (126 MHz, CDCl₃) δ 170.76, 150.22, 138.98, 138.58, 137.57, 135.46, 134.34, 128.72, 128.46, 127.12, 126.62, 123.55, 113.52, 113.22, 108.13, 102.98, 86.94, 53.95, 45.70, 26.25, 24.10, 21.21, 18.32. HRMS calcd for C₃₀H₃₄N₃Pd⁺ (M−Cl⁻) 542.1782, found 542.1788.

### Synthesis of cinnamyl[2-(2,6-Dibenzhydryl-4-methylphenyl)-5-(piperidin-1-yl)imidazo[1,5-a]pyridin-3-ylidene]chloropalladium(II) (34)

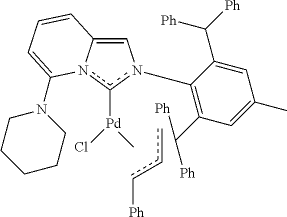

An oven-dried flask equipped with a stir bar was charged with the corresponding NHC·HCl salt (9) (290 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (265 mg, 75%). ¹H NMR (500 MHz, CDCl₃) δ 7.41 (d, J=7.6 Hz, 2H), 7.26 (t, J=7.5 Hz, 2H), 7.22-7.06 (m, 13H), 7.01 (dd, J=10.2, 5.3 Hz, 7H), 6.79 (s, 2H), 6.75-6.69 (m, 4H), 6.55 (dd, J=8.9, 7.0 Hz, 1H), 6.36 (d, J=9.0 Hz, 1H), 6.10 (d, J=6.8 Hz, 1H), 5.54 (s, 2H), 5.43 (s, 1H), 5.22 (dt, J=12.6, 9.1 Hz, 1H), 4.72 (d, J=12.8 Hz, 1H), 3.49 (s, 2H), 2.58 (s, 2H), 2.20 (s, 3H), 2.11 (d, J=8.7 Hz, 2H), 1.81 (d, J=85.5 Hz, 6H). ¹³C NMR (126 MHz, CDCl₃) δ 170.59, 149.54, 144.17, 142.35, 142.06, 138.71, 138.23, 137.12, 132.11, 130.15, 129.17, 129.13, 128.57, 128.34, 128.10, 127.35, 126.87, 126.41, 126.18, 123.11, 116.46, 113.35, 107.52, 102.58, 88.51, 53.32, 51.24, 45.07, 26.14, 24.12, 21.97. HRMS calcd for C₅₄H₅₀N₃Pd⁺ (M−Cl⁻) 846.3034, found 846.3021.

### Synthesis of cinnamyl[2-(2,6-Diisopropylphenyl)-5-morpholinoimidazo[1,5-a]pyridin-3-ylidene]chloropalladium(II) (35)

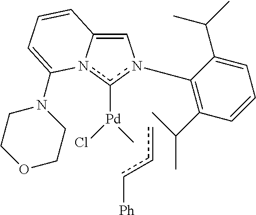

An oven-dried flask equipped with a stir bar was charged with the corresponding NHC·HCl salt (10) (176 mg, 0.44 mmol, 2.2 equiv), [{Pd(cin)Cl}₂] (103 mg, 0.20 mmol, 1.0 equiv) and KOtBu (54 mg, 0.48 mmol, 2.4 equiv), placed under a positive pressure of argon and subjected to three evacuation/backfilling cycles under high vacuum. THE (8 mL) was added and the resulting reaction mixture was stirred at room temperature for 16 h. The reaction mixture was diluted with CH₂Cl₂ and filtered. The solution was collected and concentrated. The title product was obtained by trituration from diethyl ether/hexanes (1:10 v/vol) as a yellow solid (104 mg, 42%). ¹H NMR (500 MHz, CDCl₃) δ 7.42 (d, J=7.7 Hz, 1H), 7.31-7.10 (m, 8H), 7.04 (d, J=9.0 Hz, 1H), 6.81-6.72 (m, 1H), 6.17 (d, J=6.8 Hz, 1H), 5.21 (dd, J=21.1, 9.2 Hz, 1H), 4.51 (d, J=12.7 Hz, 1H), 3.87 (d, J=4.9 Hz, 4H), 3.24 (s, 2H), 2.86 (s, 2H), 2.52 (s, 2H), 2.28 (s, 2H), 1.23 (d, J=6.7 Hz, 5H), 0.99 (d, J=6.7 Hz, 5H). ¹³C NMR (126 MHz, CDCl₃) δ 172.36, 148.60, 146.20, 137.98, 137.30, 133.58, 130.14, 129.04, 128.53, 127.95, 127.11, 126.85, 123.85, 123.55, 115.42, 113.90, 107.08, 103.30, 87.70, 67.29, 51.99, 45.08, 28.30, 26.42, 22.97. HRMS caled for C₃₂H₃₈N₃OPd⁺ (M−Cl⁻) 586.2044, found 586.2046.

### Synthesis of 2-(2,6-Diisopropylphenyl)-5-(dimethylamino)imidazo[1,5-a]pyridin-3-ylidene silver(I) chloride (36)

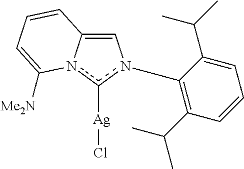

An oven-dried flask equipped with a stir bar was charged with the corresponding NHC·HCl salt (1) (107 mg, 0.3 mmol, 1.0 equiv) and Ag₂O (83.4 mg, 0.36 mmol, 1.2 equiv). The reaction mixture was placed under a positive pressure of argon and subjected to three evacuation/backfilling cycles under high vacuum. CH₂Cl₂ (6.0 ml, 0.05 M) was added and the reaction mixture was stirred at room temperature for 16 h. After the indicated time, the reaction mixture was diluted with CH₂Cl₂ (10 mL) and filtered. The solution was collected and concentrated. The title product was obtained by trituration from hexanes as a white solid (125.5 mg, 90%). ¹H NMR (500 MHz, CDCl₃) δ 7.49 (t, J=7.8 Hz, 1H), 7.33 (s, 1H), 7.28 (d, J=7.8 Hz, 2H), 7.22 (d, J=9.1 Hz, 1H), 7.00 (t, J=8.0 Hz, 1H), 6.32 (d, J=7.0 Hz, 1H), 2.93 (s, 6H), 2.25-2.13 (m, 2H), 1.24 (d, J=6.8 Hz, 6H), 1.11 (d, J=6.8 Hz, 6H). ¹³C NMR (126 MHz, CDCl₃) δ 172.86 (dd, J=262.3, 18.9 Hz), 148.66, 145.25, 136.02, 132.87 (d, J=6.5 Hz), 130.51, 124.24, 124.12, 114.39 (d, J=6.5 Hz), 112.80, 102.08, 43.89, 28.26, 24.66, 24.42. HRMS calcd for C₂₁H₂₇AgClN₃Na⁺ (M⁺+Na) 486.0837, found 486.0849.

### Synthesis of 2-(2,6-Diisopropylphenyl)-5-(dimethylamino)imidazo[1,5-a]pyridin-3-ylidene copper(I) chloride (37)

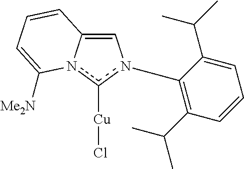

An oven-dried flask equipped with a stir bar was charged with the corresponding NHC·HCl salt (1) (107 mg, 0.3 mmol, 1.0 equiv), CuCl (29.7 mg, 0.3 mmol, 1.0 equiv) and NaOtBu (28.8 mg, 0.3 mmol, 1.0 equiv). The reaction mixture was placed under a positive pressure of argon and subjected to three evacuation/backfilling cycles under high vacuum. THE (6.0 ml, 0.05 M) was added and the reaction mixture was stirred at room temperature for 16 h. After the indicated time, the reaction mixture was diluted with CH₂Cl₂ (10 mL) and filtered. The solution was collected and concentrated. The title product was obtained by trituration from hexanes as a brown solid (107.2 mg, 85%). ¹H NMR (500 MHz, CDCl₃) δ 7.39 (t, J=7.7 Hz, 1H), 7.18 (t, J=9.5 Hz, 3H), 7.13 (d, J=9.1 Hz, 1H), 6.93-6.86 (m, 1H), 6.25 (d, J=6.7 Hz, 1H), 2.88 (s, 6H), 2.15 (dt, J=13.3, 6.5 Hz, 2H), 1.18 (d, J=6.7 Hz, 6H), 1.04 (d, J=6.8 Hz, 6H). ¹³C NMR (126 MHz, CDCl₃) δ 169.43, 148.75, 145.22, 135.84, 132.15, 130.28, 124.26, 124.09, 123.96, 122.95, 113.86, 112.91, 101.93, 43.72, 28.30, 24.75, 24.24. HRMS calcd for C₂₁H₂₇ClCuN₃Na⁺ (M⁺+Na) 442.1082, found 442.1094.

### Synthesis of 2-(2,6-diisopropylphenyl)-5-(dimethylamino)imidazo[1,5-a]pyridine-3(2H)-selenone (A1)

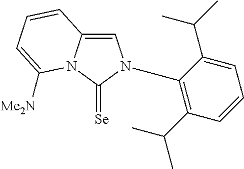

An oven-dried flask equipped with a stir bar was charged with the corresponding NHC·HCl salt (1) (107 mg, 0.3 mmol, 1.0 equiv), selenium powder (47.4 mg, 0.6 mmol, 2.0 equiv) and KOtBu (83 mg, 0.45 mmol, 1.5 equiv). The reaction mixture was placed under a positive pressure of argon and subjected to three evacuation/backfilling cycles under high vacuum. THE (6.0 ml, 0.05 M) was added and the reaction mixture was stirred at room temperature for 16 h. After the indicated time, the reaction mixture was diluted with CH₂Cl₂ (10 mL) and filtered. The solution was collected and concentrated. The residue was purified by chromatography on silica gel to afford the title product (110.5 g, 92%). ¹H NMR (500 MHz, CDCl₃) δ 7.48 (t, J=7.7 Hz, 1H), 7.38-7.24 (m, 2H), 7.09 (s, 1H), 6.88 (d, J=9.0 Hz, 1H), 6.70 (dd, J=8.8, 7.1 Hz, 1H), 5.93 (d, J=6.8 Hz, 1H), 2.84 (s, 6H), 2.31 (dt, J=13.6, 6.8 Hz, 2H), 1.30 (d, J=6.8 Hz, 6H), 1.08 (d, J=6.9 Hz, 6H). ¹³C NMR (126 MHz, CDCl₃) δ 149.66, 148.15, 145.80, 134.90, 132.80, 130.06, 124.20, 123.53, 123.47, 111.91, 111.15, 98.97, 45.32, 28.79, 24.70, 23.24. ⁷⁷Se NMR (95 MHz, CDCl₃) δ 214.65. HRMS caled for C₂₁H₂₇N₃NaSe⁺ (M⁺+Na) 424.1262, found 424.1273.

### Synthesis of 2-(2,6-Diisopropylphenyl)-5-(dimethylamino)imidazo[1,5-a]pyridin-3-ylidene(cyclooctadiene)rhodium(I) chloride (38)

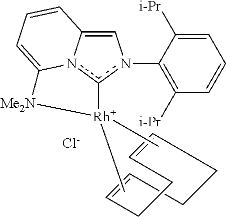

An oven-dried flask equipped with a stir bar was charged with the corresponding NHC·HCl salt (1) (107 mg, 0.3 mmol, 1.0 equiv), [Rh(cod)Cl]₂ (74 mg, 0.15 mmol, 0.5 equiv) and KOtBu (47.1 mg, 0.42 mmol, 1.4 equiv). The reaction mixture was placed under a positive pressure of argon and subjected to three evacuation/backfilling cycles under high vacuum. THE (6.0 ml, 0.05 M) was added and the reaction mixture was stirred at room temperature for 16 h. After the indicated time, the reaction mixture was diluted with CH₂Cl₂ (10 mL) and filtered. The solution was collected and concentrated. The residue was purified by chromatography on silica gel (CH₂Cl₂/MeOH=20/1) to afford the title product (129.5 mg, 76%). ¹H NMR (500 MHz, CDCl₃) δ 7.52 (t, J=7.8 Hz, 1H), 7.32 (d, J=7.8 Hz, 2H), 7.12 (d, J=12.1 Hz, 2H), 6.91 (dd, J=9.0, 7.0 Hz, 1H), 6.69 (d, J=6.9 Hz, 1H), 4.67 (s, 2H), 3.26 (s, 2H), 3.20 (s, 6H), 2.59 (dt, J=13.5, 6.7 Hz, 2H), 2.20 (s, 2H), 1.98 (s, 2H), 1.70 (s, 2H), 1.58 (d, J=7.0 Hz, 2H), 1.34 (d, J=6.8 Hz, 6H), 1.08 (d, J=6.8 Hz, 6H). ¹³C NMR (126 MHz, CDCl₃) δ 168.99 (d, J=51.0 Hz), 150.31, 145.97, 135.70, 132.18, 130.29, 128.66, 124.62, 123.83, 115.47, 114.62, 103.79, 94.78, 65.69 (d, J=18.4 Hz), 47.69, 32.71, 28.38, 28.27, 25.98, 23.14. HRMS calcd for C₂₉H₃₉N₃Rh⁺ (M−Cl⁻) 532.2194, found 532.2189.

### Synthesis of 2-(2,6-Diisopropylphenyl)-5-(dimethylamino)imidazo[1,5-a]pyridin-3-ylidene(cyclooctadiene)rhodium(I) triflate (39)

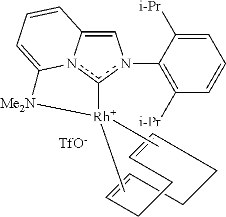

An oven-dried flask equipped with a stir bar was charged with the corresponding [Rh(NHC)(cod)]C₁ complex (38) (113.6 mg, 0.2 mmol, 1.0 equiv), AgOTf (51.4 mg, 0.2 mmol, 1.0 equiv). The reaction mixture was placed under a positive pressure of argon and subjected to three evacuation/backfilling cycles under high vacuum. CH₂Cl₂ (2.0 ml, 0.1 M) was added and the reaction mixture was stirred at room temperature for 1 h. After the indicated time, the reaction mixture was diluted with CH₂Cl₂ (10 mL) and filtered. The solution was collected and concentrated. The title product was obtained as a yellow solid (109 mg, 80%). ¹H NMR (500 MHz, CDCl₃) δ 7.53 (t, J=7.8 Hz, 1H), 7.40 (dd, J=6.1, 3.7 Hz, 1H), 7.30 (s, 1H), 7.28 (s, 1H), 7.21 (s, 1H), 7.20-7.17 (m, 2H), 4.61 (s, 2H), 3.73 (d, J=2.9 Hz, 2H), 3.26 (s, 6H), 2.30 (ddd, J=20.4, 13.8, 6.2 Hz, 4H), 2.17-2.08 (m, 2H), 1.93 (dd, J=13.9, 5.9 Hz, 2H), 1.79 (dd, J=14.1, 6.9 Hz, 2H), 1.37 (d, J=6.8 Hz, 6H), 1.11 (d, J=6.8 Hz, 6H). ¹³C NMR (126 MHz, CDCl₃) δ 161.16, 160.72, 148.48, 144.93, 133.67, 131.34, 130.88, 126.18, 124.29, 116.56, 115.26, 105.09, 98.55, 98.49, 73.00, 72.89, 49.83, 31.44, 28.51, 28.47, 26.07, 22.60. ¹⁹F NMR (471 MHz, CDCl₃) δ-78.00. HRMS calcd for C₂₉H₃₉N₃Rh⁺ (M−OTf⁻) 532.2194, found 532.2202.

### Synthesis of 2-(2,6-Diisopropylphenyl)-5-(dimethylamino)imidazo[1,5-a]pyridin-3-ylidene(bis(carbonyl))rhodium(I) trilate (40

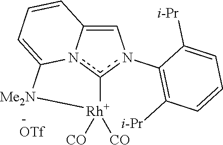

An oven-dried flask equipped with a stir bar was charged with the corresponding [Rh(NHC)(cod)]OTf complex (39) (68.2 mg, 0.1 mmol, 1.0 equiv) and 5 ml THF. CO gas was bubbled through the reaction and the reaction mixture was stirred at room temperature for 2 hours. The resulting mixture was concentrated and the title product was obtained as a yellow solid (56.6 mg, 90%). ¹H NMR (500 MHz, CDCl₃) δ 7.67-7.54 (m, 4H), 7.36 (d, J=7.5 Hz, 3H), 3.71-3.56 (m, 6H), 2.23-2.14 (m, 2H), 1.29 (d, J=6.6 Hz, 6H), 1.18 (d, J=6.6 Hz, 6H). ¹³C NMR (126 MHz, CDCl₃) δ 185.12 (d, J=57.7 Hz), 182.70 (d, J=73.5 Hz), 163.97 (d, J=48.4 Hz), 146.42, 145.44, 134.00, 131.90, 131.74, 127.17, 124.51, 117.77, 115.47, 108.25, 54.05, 28.47, 24.71, 24.13. ¹⁹F NMR (471 MHz, CDCl₃) δ-78.23. HRMS caled for C₂₃H₂₇N₃O₂Rh⁺ (M−OTf⁻) 480.1153, found 480.1177.

### Synthesis of 2-(2,6-Diisopropylphenyl)-5-(dimethylamino)imidazo[1,5-a]pyridin-3-ylidene gold(I) bis(trifluoromethanesulfonyl)amide (41)

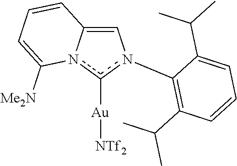

An oven-dried flask equipped with a stir bar was charged with the corresponding Au(NHC)Cl complex (16) (55.5 mg, 0.1 mmol, 1.0 equiv) and AgNTf₂ (38.8 mg, 0.1 mmol, 1.0 equiv). The reaction mixture was placed under a positive pressure of argon and subjected to three evacuation/backfilling cycles under high vacuum. CH₂Cl₂ (1.0 mL, 0.1 M) was added and the reaction mixture was stirred at room temperature for 0.5 h. After the indicated time, the reaction mixture was diluted with CH₂Cl₂ (5 mL), filtered and concentrated. The title product was obtained as a brown solid (68 mg, 85%). ¹H NMR (500 MHz, CDCl₃) δ 7.54 (t, J=7.8 Hz, 1H), 7.37 (s, 1H), 7.30 (d, J=7.8 Hz, 2H), 7.25-7.21 (m, 1H), 7.05 (dd, J=9.1, 7.1 Hz, 1H), 6.37 (dd, J=7.0, 0.7 Hz, 1H), 2.90 (s, 6H), 2.16 (dt, J=13.7, 6.9 Hz, 2H), 1.29 (d, J=6.9 Hz, 6H), 1.12 (d, J=6.9 Hz, 6H). ¹³C NMR (126 MHz, CDCl₃) δ 156.42, 148.35, 145.07, 135.44, 132.84, 130.70, 124.72, 124.12, 119.15 (dd, J=646.9, 323.4 Hz), 114.47, 112.80, 103.20, 43.75, 28.51, 24.86, 23.55. ¹⁹F NMR (471 MHz, CDCl₃) δ-75.25. HRMS calcd for C₂₃H₃₀AuN₄⁺ (M−NTf₂⁻+CH₃CN) 559.2131, found 559.2147.

### Synthesis of 2-(2,6-diisopropylphenyl)-5-(morpholin-4-yl)imidazo[1,5-a]pyridin-3-ylidene gold(I) chloride (42)

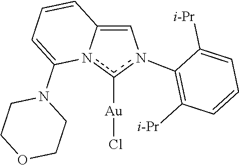

An oven-dried flask equipped with a stir bar was charged with the corresponding NHC·HCl salt (10) (120 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was directly used for next step without further purification. The above residue silver complex was dissolved in DCM (4 mL), AuClSMe₂ (90 mg, 0.3 mmol, 1.0 equiv) was added. The mixture was stirred at room temperature for 6 hours. The solution was filtered and concentrated. The residue was purified by chromatography on silica gel to afford the title product (107 mg, 60%). ¹H NMR (500 MHz, Chloroform-d) δ 7.51 (t, J=7.8 Hz, 1H), 7.29 (d, J=4.2 Hz, 2H), 7.27 (s, 1H), 7.24 (dd, J=9.0, 1.0 Hz, 1H), 7.01 (dd, J=9.1, 7.0 Hz, 1H), 6.38 (dd, J=7.1, 1.1 Hz, 1H), 4.47 (td, J=11.6, 2.3 Hz, 2H), 3.91 (dd, J=11.9, 3.0 Hz, 2H), 3.43-3.37 (m, 2H), 3.00 (td, J=11.5, 3.1 Hz, 2H), 2.20 (p, J=6.8 Hz, 2H), 1.31 (d, J=6.8 Hz, 6H), 1.11 (d, J=6.9 Hz, 6H). ¹³C NMR (126 MHz, CDCl₃) δ 164.06, 147.35, 145.12, 135.69, 132.41, 130.69, 124.18, 124.12, 114.19, 113.80, 103.75, 66.22, 52.29, 28.48, 24.53, 24.29. HRMS calcd for C₂₃H₂₉AuN₃O⁺ (M−Cl⁻) 560.1971, found 560.1950.

### Synthesis of 2-(2,4,6-trimethylphenyl)-5-(morpholin-4-yl)imidazo[1,5-a]pyridin-3-ylidene gold(I) chloride (43)

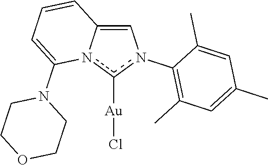

An oven-dried flask equipped with a stir bar was charged with the corresponding NHC·HCl salt (11) (107 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was directly used for next step without further purification. The above residue silver complex was dissolved in DCM (4 mL), AuClSMe₂ (90 mg, 0.3 mmol, 1.0 equiv) was added. The mixture was stirred at room temperature for 6 hours. The solution was filtered and concentrated. The residue was purified by chromatography on silica gel to afford the title product (91 mg, 65%). ¹H NMR (500 MHz, Chloroform-d) δ 7.28 (d, J=2.3 Hz, 1H), 7.25 (dd, J=9.2, 1.0 Hz, 1H), 7.03-6.95 (m, 3H), 6.37 (dd, J=7.0, 1.0 Hz, 1H), 4.47 (td, J=11.6, 2.2 Hz, 2H), 3.94-3.85 (m, 2H), 3.45-3.34 (m, 2H), 2.99 (td, J=11.5, 3.1 Hz, 2H), 2.34 (s, 3H), 1.98 (s, 6H). ¹³C NMR (126 MHz, CDCl₃) δ 162.77, 147.26, 139.68, 136.34, 134.18, 132.78, 129.39, 123.87, 114.03, 112.99, 103.80, 66.23, 52.28, 21.19, 17.84. HRMS calcd for C₂₁H₂₃AuClN₃O⁺ (M+H⁺) 554.1268, found 554.1291

### Example 3: Representative Decagram-Scale Synthesis of NHC (e.g., 2-(2,6-Diisopropylphenyl)-5-(dimethylamino)imidazo[1,5-a]pyridin-2-ium chloride) (1)

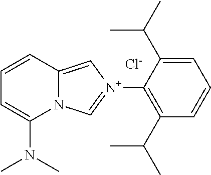 [SMILES: CC(C)c1cccc(C(C)C)c1-[n+]1cc2cccc(N(C)C)n2c1 ; [Cl-]]

### Step 1. Synthesis of 6-Bromo-N,N-dimethylpyridin-2-amine (S1)

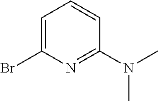

An oven-dried 500 mL round-bottomed flask equipped with a stir bar was charged with 2,6-dibromopyridine (23.69 g, 100 mmol, 1.0 equiv), dimethylamine solution (63.3 ml, 500 mmol, 5.0 equiv, 40 wt % in H₂O), K₂CO₃ (15.2 g, 110 mmol, 1.1 equiv) and CH₃CN (100 mL). The mixture was refluxed at 100° C. until the starting material was fully consumed (48-72 hours). Then, the mixture was cooled down to room temperature and extracted with EtOAc (100 mL×2). The organic layers were combined, dried over Na₂SO₄, filtered, and concentrated to afford 6-bromo-N,N-dimethylpyridin-2-amine (19.7 g, 98%). ¹H NMR (500 MHz, CDCl₃) δ 7.23 (t, J=7.9 Hz, 1H), 6.65 (d, J=7.4 Hz, 1H), 6.37 (d, J=8.4 Hz, 1H), 3.08-3.03 (s, 6H). ¹³C NMR (126 MHz, CDCl₃) δ 159.21, 140.13, 139.11, 114.19, 103.87, 37.92.

### Step 2. Synthesis of 6-(Dimethylamino)picolinaldehyde (S2)

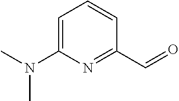

An oven-dried 500 mL round-bottomed flask equipped with a stir bar was charged with 6-bromo-N,N-dimethylpyridin-2-amine (19.7 g, 98 mmol, 1.0 equiv) and dry THE (200 mL). n-BuLi (2.5 M in hexane, 59 mL, 1.5 equiv) was slowly added at −78° C. under argon and the resulting mixture was stirred at −78° C. for 1 hour. After the indicated time, anhydrous DMF (9.1 mL, 117.6 mmol, 1.2 equiv) was added dropwise at −78° C. and the reaction was stirred for 1 hour at −78° C. under argon. After the indicated time, the reaction mixture was quenched with NaHCO₃ (50 mL, sat., aq.), diluted with EtOAc (300 mL), washed with H₂O (100 mL×2) and brine (50 mL). The organic layers were collected, dried over Na₂SO₄, filtered, and concentrated. The resulting brown oil was used directly in the next step (75% yield, ¹H NMR).

### Step 3. Synthesis of (E)-6-(((2,6-Diisopropylphenyl)imino)methyl)-N,N-dimethylpyridin-2-amine (S3)

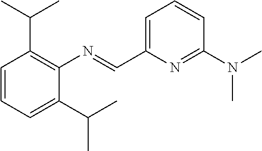 [SMILES: CC(C)c1cccc(C(C)C)c1/N=C/c1cccc(N(C)C)n1]

An oven-dried 500 mL round-bottomed flask equipped with a stir bar was charged with crude 6-(dimethylamino)picolinaldehyde (73.5 mmol based on ¹H NMR yield), 2,6-diisopropylaniline (13.03 g, 73.5 mmol, 1.0 equiv) and dry EtOH (150 mL) and the reaction mixture was stirred at 90° C. for 24 hours. After the indicated time, the reaction mixture was concentrated, cooled down to room temperature and settled overnight to form crystals. The resulting crystals were filtered and recrystallized from CH₂Cl₂/hexane 3 times to afford analytically pure imine as a yellow solid (17.29 g, 76%). ¹H NMR (500 MHz, CDCl₃) δ 8.28 (s, 1H), 7.76-7.59 (m, 2H), 7.35-7.15 (m, 3H), 6.69 (d, J=8.0 Hz, 1H), 3.21 (s, 6H), 3.12 (dt, J=13.7, 6.8 Hz, 2H), 1.29 (d, J=6.9 Hz, 12H). ¹³C NMR (126 MHz, CDCl₃) δ 144.91, 144.05, 132.33, 131.98, 130.74, 127.34, 124.49, 123.67, 117.23, 113.60, 105.27, 41.91, 28.68, 24.54, 24.20. HRMS caled for C₂₀H₂₈N₃⁺ (M+H⁺) 310.2278, found 310.2293.

### Step 4. Synthesis of 2-(2,6-Diisopropylphenyl)-5-(dimethylamino)imidazo[1,5-a]pyridin-2-ium chloride (1)

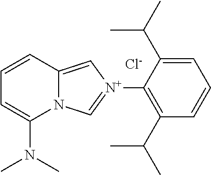 [SMILES: CC(C)c1cccc(C(C)C)c1-[n+]1cc2cccc(N(C)C)n2c1 ; [Cl-]]

An oven-dried 250 mL round-bottomed flask equipped with a stir bar was charged with (E)-6-(((2,6-Diisopropylphenyl)imino)methyl)-N,N-dimethylpyridin-2-amine (11.76 g, 38 mmol, 1.0 equiv), paraformaldehyde (1.71 g, 57 mmol, 1.5 equiv) and EtOH (70 mL). HCl (4.0 M in dioxane, 19 mL, 2.0 equiv) was added slowly at 0° C., the reaction was warmed up to room temperature and stirred at 70° C. for 36 hours. After the indicated time, the reaction was cooled down to room temperature, neutralized with NaHCO₃, filtered and concentrated. The residue was dissolved in 100 mL CH₂Cl₂ and mixed with 100 g of silica gel. Then, CH₂Cl₂ was evaporated and the resulting solid material washed with CH₂Cl₂ (300 mL) and filtered using filtration funnel. The CH₂Cl₂ washing and filtration procedure was repeated 4 times. The organic layers were discarded. The solid material was washed with CH₂Cl₂/MeOH (20/1, 200 mL) and filtered. The CH₂Cl₂/MeOH washing and filtration procedure was repeated 4 times. The CH₂Cl₂/MeOH organic layers were combined and concentrated. The resulting solid was dried in an oven at 175° F. for 16 hours. The final product was obtained as a yellow solid (10.88 g, 80%, purity>98%).

### Example 4: Synthesis of Imidazo[1,5-a]quinoline N-Heterocyclic Carbene (NHC) Ligands General Procedure

### Step 1—Synthesis of N,N-disubstituted-2-nitroaniline (4-2)

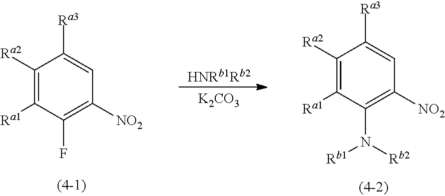

An oven-dried 500 mL round-bottomed flask equipped with a stir bar was charged with (4-1) (28.2 g, 200 mmol, 1.0 equiv), K₂CO₃ (33.2 g, 240 mmol, 1.2 equiv), amine (HNR^{b1}R^{b2}) (300 mmol, 1.5 equiv) and acetonitrile (200 mL) at room temperature. The mixture was refluxed for 24 hours. After the indicated time, the reaction mixture was cooled down to room temperature, filtered, and concentrated. The residue was dissolved in 200 mL CH₂Cl₂, washed with H₂O (2×100 mL) and brine (1×50 mL). The organic layers were combined, dried over Na₂SO₄, filtered, and concentrated. The residue was directly used for the next step without further purification.

### Step 2 Synthesis of N¹,N¹-disubstituted benzene-1,2-diamine (4-3)

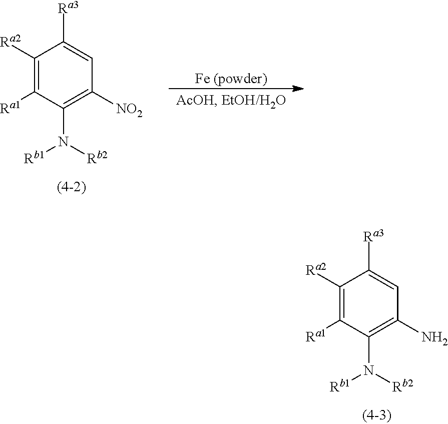

An oven-dried 1000 mL round-bottomed flask equipped with a stir bar was charged with (4-2), iron powder (58.85 g, 1000 mmol, 5.0 equiv), acetic acid (400 mmol, 2.0 equiv) and EtOH/H₂O (500 mL/50 mL). The mixture was refluxed for 24 hours. After the indicated time, the reaction was cooled down to room temperature, filtered and concentrated. The residue was dissolved in 400 mL ethyl acetate and washed with H₂O (2×200 mL) and brine (1×50 mL). The organic layers were combined, dried over Na₂SO₄, filtered, and concentrated. The residue was directly used for the next step without further purification.

### Step 3 Synthesis of N,N-disubstituted-2-methylquinolin-8-amine (4-4)

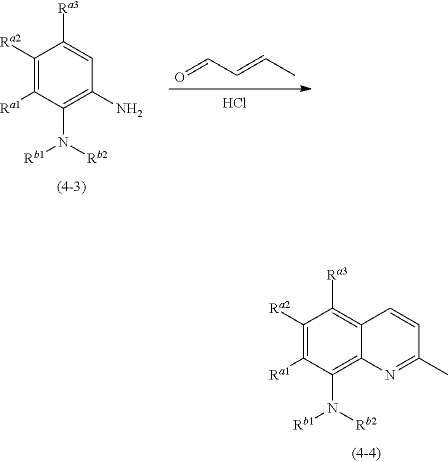

An oven-dried 500 mL round-bottomed flask equipped with a stir bar was charged with (4-3). 6 N HCl (250 mL) was added slowly at 0° C., then, crotonaldehyde (33.1 mL, 400 mmol, 2.0 equiv) was slowly added at 0° C., followed by addition of toluene (150 mL). The mixture was stirred and refluxed for 4 hours. After the indicated time, the reaction was cooled down to room temperature and was neutralized with 6 N NaOH aqueous solution. The resulting mixture was extracted with dichloromethane (2×500 mL). The organic layers were combined, dried over Na₂SO₄, filtered, and concentrated. The residue was purified by chromatography on silica gel to afford the title product.

### Step 4 Synthesis of 8-(disubstituted amino)quinoline-2-carbaldehyde (4-5)

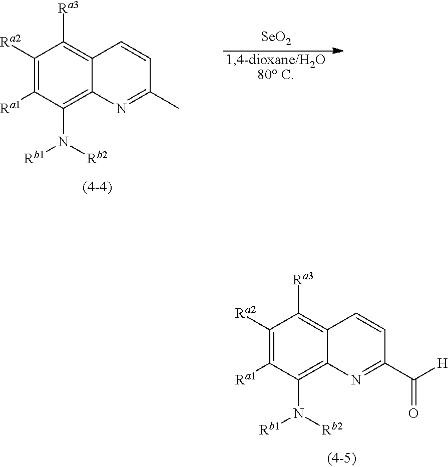

An oven-dried 250 mL round-bottomed flask equipped with a stir bar was charged with SeO₂ (13.3 g, 120 mmol, 1.2 equiv), 1,4-dioxane/H₂O (120 mL/1.2 mL). The mixture was stirred at 80° C. for 0.5 hour. Then (4-4) (100 mmol, 1.0 equiv) was added. The mixture was stirred at 80° C. for 3 hours. After the indicated time, the reaction was cooled down to room temperature and filtered, concentrated. The residue was purified by chromatography on silica gel to afford the title product.

### Step 5 Synthesis of 2-(substituted phenyl)-5-(disubstituted amino)imidazo[1,5-a]quinoline-2-ium chloride (4-7)

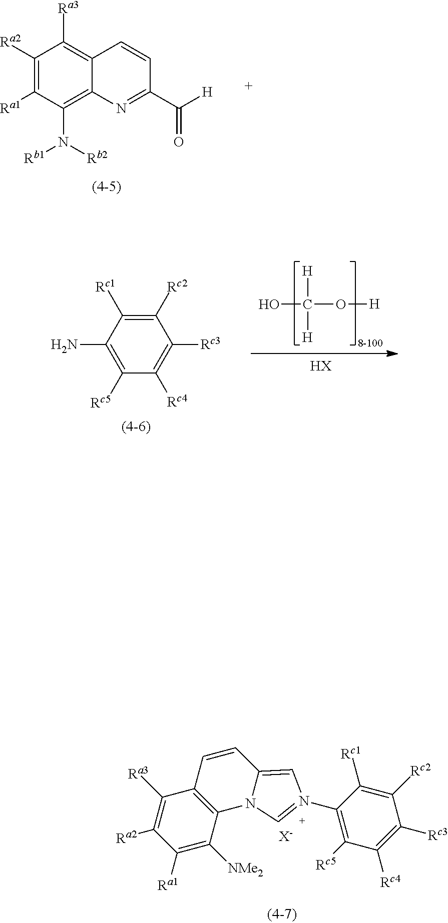

An oven-dried 100 mL round-bottomed flask equipped with a stir bar was charged with (4-5) (10 mmol, 1.0 equiv), (4-6) (10 mmol, 1.0 equiv), paraformaldehyde (450 mg, 15 mmol, 1.5 equiv) and toluene (25 mL). HCl (4.0 M in dioxane, 5.0 mL, 2.0 equiv) was added dropwise into the reaction at room temperature. The mixture was stirred at 110° C. for 24 hours. After the indicated time, the reaction was cooled down to room temperature and concentrated. The residue was purified by chromatography on silica gel (CH₂Cl₂/MeOH=20/1) to afford the title product.

The following compounds were prepared according to Step 3 of the general procedure:

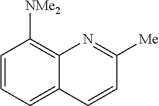 [SMILES: Cc1ccc2cccc(N(C)C)c2n1]

N,N-2-trimethylquinolin-8-amine (44). According to the general procedure, the title product was obtained in 55% yield. ¹H NMR (500 MHz, Chloroform-d) δ 7.99 (d, J=8.3 Hz, 1H), 7.40-7.33 (m, 2H), 7.26 (d, J=8.3 Hz, 1H), 7.14-7.06 (m, 1H), 3.13 (s, 6H), 2.80 (s, 3H). ¹³C NMR (126 MHz, CDCl₃) δ 156.35, 149.84, 142.14, 136.48, 127.70, 125.64, 121.54, 120.52, 115.45, 25.77.

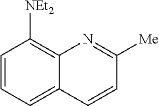

N,N-diethyl-2-methylquinolin-8-amine (45). According to the general procedure, the title product was obtained in 58% yield. ¹H NMR (500 MHz, Chloroform-d) δ 7.96 (d, J=8.4 Hz, 1H), 7.35-7.27 (m, 2H), 7.22 (d, J=8.3 Hz, 1H), 7.10-7.04 (m, 1H), 3.57 (q, J=7.0 Hz, 4H), 2.74 (s, 3H), 1.16 (t, J=7.1 Hz, 6H). ¹³C NMR (126 MHz, CDCl₃) δ 156.06, 147.07, 142.82, 136.45, 127.88, 125.32, 121.43, 119.99, 117.68, 46.78, 25.74, 12.06. HRMS calcd for C₄₂H₅₅N₆⁺ (3M+H⁺) 643.4483, found 643.4509.

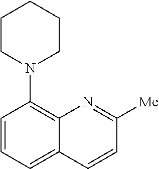 [SMILES: Cc1ccc2cccc(N3CCCCC3)c2n1]

2-methyl-8-(piperidin-1-yl)quinoline (46). According to the general procedure, the title product was obtained in 68% yield. ¹H NMR (500 MHz, Chloroform-d) δ 7.96 (d, J=8.3 Hz, 1H), 7.39-7.31 (m, 2H), 7.22 (d, J=8.4 Hz, 1H), 7.10 (dd, J=6.5, 2.5 Hz, 1H), 3.33 (t, J=5.3 Hz, 4H), 2.74 (s, 3H), 1.91 (p, J=5.6 Hz, 4H), 1.67 (q, J=6.2 Hz, 2H). ¹³C NMR (126 MHz, CDCl₃) δ 156.46, 150.06, 142.19, 136.49, 127.68, 125.74, 121.37, 120.80, 115.86, 53.66, 26.21, 25.91, 24.75. HRMS calcd for C₃₀H₃₆KN₄⁺ (2M+K⁺) 491.2572, found 491.2587.

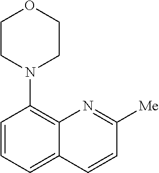 [SMILES: Cc1ccc2cccc(N3CCOCC3)c2n1]

4-(2-methylquinolin-8-yl)morpholine (47). According to the general procedure, the title product was obtained in 66% yield. ¹H NMR (500 MHz, Chloroform-d) δ 7.99 (d, J=8.4 Hz, 1H), 7.43-7.34 (m, 2H), 7.25 (d, J=8.4 Hz, 1H), 7.09 (dd, J=6.9, 2.1 Hz, 1H), 4.08-4.02 (m, 4H), 3.43 (t, J=4.6 Hz, 4H), 2.73 (s, 3H). ¹³C NMR (126 MHz, CDCl₃) δ 156.80, 148.44, 141.87, 136.68, 127.76, 125.75, 121.65, 121.62, 115.63, 67.20, 52.55, 25.85. HRMS calcd for C₄₂H₄₉N₆O₃⁺ (3M+H⁺) 685.3861, found 685.3896.

The following compounds were prepared according to Step 4 of the general procedure:

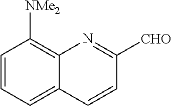 [SMILES: CN(C)c1cccc2ccc(C=O)nc12]

8-(dimethylamino)quinoline-2-carbaldehyde (48). According to the general procedure, the title product was obtained in 75% yield. ¹H NMR (500 MHz, Chloroform-d) δ 10.24 (d, J=0.9 Hz, 1H), 8.24 (d, J=8.4 Hz, 1H), 8.00 (d, J=8.4 Hz, 1H), 7.56 (t, J=7.9 Hz, 1H), 7.41 (dd, J=8.2, 1.3 Hz, 1H), 7.17-7.11 (m, 1H), 3.22 (s, 6H).

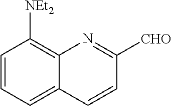

8-(diethylamino)quinoline-2-carbaldehyde (49). According to the general procedure, the title product was obtained in 78% yield. ¹H NMR (500 MHz, Chloroform-d) δ 10.21 (d, J=0.9 Hz, 1H), 8.21-8.16 (m, 1H), 7.96 (d, J=8.4 Hz, 1H), 7.52 (t, J=7.9 Hz, 1H), 7.31 (dd, J=8.0, 1.2 Hz, 1H), 7.10 (dd, J=7.8, 1.3 Hz, 1H), 3.67 (q, J=7.0 Hz, 4H), 1.28 (t, J=7.0 Hz, 6H). ¹³C NMR (126 MHz, CDCl₃) δ 193.79, 149.52, 148.76, 142.13, 137.56, 132.09, 129.65, 118.48, 116.84, 116.47, 47.02, 12.54. HRMS calcd for C₁₄H₁₇N₂O⁺ (M+H⁺) 229.1335, found 229.1338.

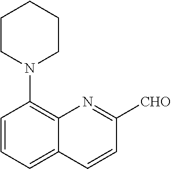 [SMILES: O=Cc1ccc2cccc(N3CCCCC3)c2n1]

8-(piperidin-1-yl)quinoline-2-carbaldehyde (50). According to the general procedure, the title product was obtained in 85% yield. ¹H NMR (500 MHz, Chloroform-d) δ 10.19 (s, 1H), 8.24 (d, J=8.4 Hz, 1H), 7.99 (d, J=8.4 Hz, 1H), 7.56 (t, J=7.9 Hz, 1H), 7.44 (d, J=8.1 Hz, 1H), 7.20 (d, J=7.6 Hz, 1H), 3.42 (t, J=5.4 Hz, 4H), 1.94 (p, J=5.7 Hz, 4H), 1.71 (p, J=5.9 Hz, 2H). ¹³C NMR (126 MHz, CDCl₃) δ 194.00, 151.51, 150.05, 142.25, 137.81, 131.75, 129.84, 120.51, 116.94, 116.74, 53.76, 26.18, 24.59. HRMS calcd for C₄₅H₄₉N₆O₃⁺ (M+H⁺) 721.3861, found 721.3900.

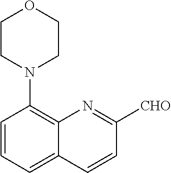 [SMILES: O=Cc1ccc2cccc(N3CCOCC3)c2n1]

8-morpholinoquinoline-2-carbaldehyde (51). According to the general procedure, the title product was obtained in 71% yield. ¹H NMR (500 MHz, Chloroform-d) δ 10.13 (d, J=0.9 Hz, 1H), 8.21 (dd, J=8.4, 0.9 Hz, 1H), 7.96 (d, J=8.4 Hz, 1H), 7.54 (t, J=7.9 Hz, 1H), 7.45 (dd, J=8.1, 1.3 Hz, 1H), 7.15 (dd, J=7.6, 1.3 Hz, 1H), 4.05-4.00 (m, 4H), 3.49-3.43 (m, 4H). ¹³C NMR (126 MHz, CDCl₃) δ 193.48, 150.19, 149.93, 141.92, 138.02, 131.71, 129.78, 121.42, 117.11, 116.54, 67.06, 52.54. HRMS caled for C₂₈H₂₉N₄O₄⁺ (M+H⁺) 485.2183, found 485.2196.

The following compounds were prepared according to Step 5 of the general procedure:

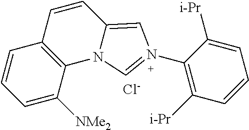

2-(2,6-diisopropylphenyl)-9-(dimethylamino)imidazo[1,5-a]quinolin-2-ium chloride (52). According to the general procedure, the title product was obtained in 88% yield. ¹H NMR (500 MHz, Chloroform-d) δ 10.56 (d, J=1.8 Hz, 1H), 9.10 (d, J=1.9 Hz, 1H), 8.58 (d, J=9.6 Hz, 1H), 7.72 (dq, J=6.6, 3.4, 2.9 Hz, 3H), 7.67-7.60 (m, 2H), 7.40 (d, J=7.8 Hz, 2H), 2.79 (s, 6H), 2.26 (p, J=6.8 Hz, 2H), 1.26 (d, J=6.8 Hz, 6H), 1.18 (d, J=6.8 Hz, 6H). ¹³C NMR (126 MHz, CDCl₃) δ 163.74, 145.59, 145.29, 132.22, 131.40, 130.71, 129.66, 129.29, 127.75, 127.61, 126.70, 124.80, 124.36, 123.19, 119.12, 117.89, 44.82, 28.80, 24.73, 24.17. HRMS calcd for C₂₅H₃₀N₃⁺ (M−Cl⁻) 372.2434, found 372.2456.

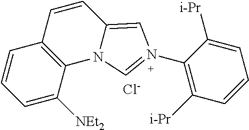

9-(diethylamino)-2-(2,6-diisopropylphenyl)imidazo[1,5-a]quinolin-2-ium chloride (53). According to the general procedure, the title product was obtained in 85% yield. ¹H NMR (500 MHz, Chloroform-d) δ 11.01 (s, 1H), 8.18 (s, 1H), 7.88 (d, J=9.6 Hz, 1H), 7.78-7.61 (m, 5H), 7.40 (d, J=7.9 Hz, 2H), 3.19 (dq, J=13.9, 7.0 Hz, 2H), 3.08 (dq, J=14.1, 7.2 Hz, 2H), 2.22 (p, J=6.8 Hz, 2H), 1.19 (dd, J=21.0, 6.8 Hz, 12H), 0.96 (t, J=7.1 Hz, 6H). ¹³C NMR (126 MHz, CDCl₃) δ 145.22, 141.77, 132.39, 131.20, 130.37, 130.00, 129.42, 128.63, 127.38, 127.35, 127.23, 126.74, 124.87, 117.33, 115.88, 48.98, 28.76, 24.55, 23.89, 11.55. HRMS calcd for C₂₇H₃₄N₃⁺ (M−Cl⁻) 400.2747, found 400.2760.

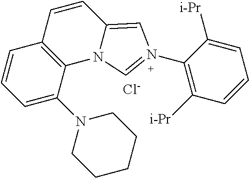

2-(2,6-diisopropylphenyl)-9-(piperidin-1-yl)imidazo[1,5-a]quinolin-2-ium chloride (54). According to the general procedure, the title product was obtained in 87% yield. ¹H NMR (500 MHz, Chloroform-d) δ 10.79-10.67 (m, 1H), 8.94 (d, J=1.8 Hz, 1H), 8.43 (d, J=9.6 Hz, 1H), 7.63 (s, 3H), 7.54 (q, J=9.3, 8.6 Hz, 2H), 7.30 (d, J=7.9 Hz, 2H), 2.96 (d, J=11.7 Hz, 2H), 2.81 (t, J=10.9 Hz, 2H), 2.13 (p, J=6.8 Hz, 2H), 1.75 (dd, J=9.8, 6.1 Hz, 3H), 1.40-1.28 (m, 3H), 1.14 (d, J=6.7 Hz, 6H), 1.05 (d, J=6.7 Hz, 6H). ¹³C NMR (126 MHz, CDCl₃) δ 145.28, 145.08, 132.29, 131.34, 130.47, 129.79, 129.16, 127.81, 127.48, 126.77, 124.75, 124.27, 124.03, 118.87, 117.43, 54.10, 28.74, 26.18, 24.65, 23.97, 23.22. HRMS calcd for C₂₈H₃₄N₃⁺ (M−Cl⁻) 412.2747, found 412.2757.

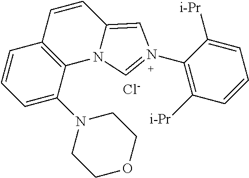

2-(2,6-diisopropylphenyl)-9-morpholinoimidazo[1,5-a]quinolin-2-ium chloride (55). According to the general procedure, the title product was obtained in 76% yield. ¹H NMR (500 MHz, Chloroform-d) δ 10.88 (s, 1H), 9.00 (s, 1H), 8.44 (d, J=9.4 Hz, 1H), 7.81-7.73 (m, 3H), 7.61 (dd, J=8.8, 6.8 Hz, 2H), 7.35 (d, J=7.8 Hz, 2H), 3.94 (dd, J=11.8, 2.7 Hz, 2H), 3.53 (t, J=11.4 Hz, 2H), 3.18 (td, J=11.8, 2.9 Hz, 2H), 2.91 (d, J=11.8 Hz, 2H), 2.17 (q, J=6.7 Hz, 2H), 1.17 (d, J=6.6 Hz, 6H), 1.10 (d, J=6.7 Hz, 6H). ¹³C NMR (126 MHz, CDCl₃) δ 144.94, 143.52, 132.33, 131.36, 130.27, 130.04, 128.71, 127.80, 127.56, 127.54, 124.72, 124.34, 124.29, 118.91, 117.29, 66.60, 52.99, 28.64, 24.61, 23.92. HRMS calcd for C₂₇H₃₂N₃⁺ (M−Cl⁻) 414.2540, found 414.2546.

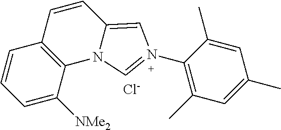

9-(dimethylamino)-2-mesitylimidazo[1,5-a]quinolin-2-ium chloride (56). According to the general procedure, the title product was obtained in 84% yield. ¹H NMR (500 MHz, Chloroform-d) δ 10.53 (d, J=1.8 Hz, 1H), 8.94 (d, J=1.8 Hz, 1H), 8.34 (d, J=9.6 Hz, 1H), 7.67-7.60 (m, 3H), 7.51 (d, J=9.6 Hz, 1H), 7.05 (s, 2H), 2.75 (s, 6H), 2.35 (s, 3H), 2.06 (s, 6H). ¹³C NMR (126 MHz, CDCl₃) δ 145.64, 141.68, 134.18, 131.31, 131.29, 129.95, 129.59, 129.24, 127.48, 127.34, 126.24, 124.22, 122.93, 117.89, 117.36, 44.66, 21.18, 17.65. HRMS calcd for C₂₂H₂₄N₃⁺ (M−Cl⁻) 330.1965, found 330.1978.

### Example 5: Synthesis of Imidazo[1,5-a]quinoline NHC Ligand-Metal Complexes

### Synthesis of 2-(2,6-Diisopropylphenyl)-9-(dimethylamino)imidazo[1,5-a]quinolin-3-ylidene silver(I) chloride (57)

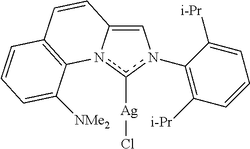

An oven-dried flask equipped with a stir bar was charged with the corresponding NHC·HCl salt (52) (122 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was obtained by trituration from hexanes as a grown solid (125 mg, 81%). ¹H NMR (500 MHz, Chloroform-d) δ 7.50 (d, J=7.8 Hz, 1H), 7.47-7.40 (m, 2H), 7.39-7.28 (m, 5H), 7.23 (d, J=9.4 Hz, 1H), 2.76 (s, 6H), 2.35 (p, J=6.9 Hz, 2H), 1.30 (d, J=6.9 Hz, 6H), 1.15 (d, J=6.9 Hz, 6H). 13C NMR (126 MHz, CDCl₃) δ 185.78 (d, J=18.6 Hz), 183.71 (d, J=18.7 Hz), 145.54, 145.32, 137.18, 135.95, 131.95 (d, J=7.2 Hz), 130.52, 128.10, 127.06, 126.41, 126.31, 124.15, 123.55, 123.01, 120.30, 115.23, 114.37 (d, J=6.8 Hz), 44.34, 28.35, 24.65, 24.38. HRMS caled for C₂₅H₂₉AgN₃⁺ (M−Cl⁻) 478.1407, found 478.1395.

### Synthesis of 2-(2,6-Diisopropylphenyl)-9-(diethylamino)imidazo[1,5-a]quinolin-3-ylidene silver(I) chloride (58)

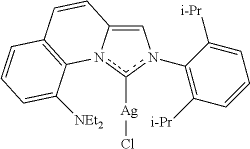

An oven-dried flask equipped with a stir bar was charged with the corresponding NHC·HCl salt (53) (131 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was obtained by trituration from hexanes as a grown solid (143 mg, 88%). ¹H NMR (500 MHz, Chloroform-d) δ 7.50 (t, J=7.8 Hz, 1H), 7.45 (dd, J=8.1, 7.3 Hz, 1H), 7.39 (ddd, J=9.1, 7.7, 1.9 Hz, 2H), 7.33 (d, J=1.7 Hz, 1H), 7.29 (d, J=7.8 Hz, 2H), 7.26 (s, 1H), 7.21 (d, J=9.4 Hz, 1H), 3.26 (dt, J=13.7, 6.8 Hz, 2H), 3.16 (dq, J=13.1, 7.2 Hz, 2H), 2.32 (p, J=6.9 Hz, 2H), 1.29 (d, J=6.9 Hz, 6H), 1.16-1.08 (m, 12H). ¹³C NMR (126 MHz, CDCl₃) δ 186.53 (d, J=18.4 Hz), 184.47 (d, J=18.6 Hz), 145.60, 141.03, 136.09, 132.12 (d, J=7.3 Hz), 130.69, 128.64, 128.34, 126.64, 126.40, 124.84, 124.46, 124.27, 115.08, 114.34 (d, J=6.7 Hz), 47.55, 28.46, 24.80, 24.35, 11.56. HRMS calcd for C₂₇H₃₃AgN₃⁺ (M−Cl⁻) 506.1720, found 506.1706.

### Synthesis of 2-(2,6-Diisopropylphenyl)-9-(piperidin-1-yl)imidazo[1,5-a]quinolin-3-ylidene silver(I) chloride (59)

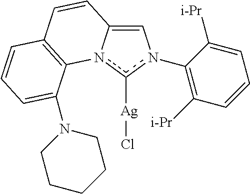

An oven-dried flask equipped with a stir bar was charged with the corresponding NHC·HCl salt (54) (134 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was obtained by trituration from hexanes as a grown solid (152 mg, 91%). ¹H NMR (500 MHz, Chloroform-d) δ 7.42 (s, 1H), 7.39-7.33 (m, 2H), 7.27-7.15 (m, 5H), 7.11 (d, J=9.4 Hz, 1H), 3.35-3.25 (m, 2H), 2.47 (td, J=11.6, 2.5 Hz, 2H), 2.34-2.15 (m, 4H), 1.74-1.64 (m, 1H), 1.59 (d, J=13.7 Hz, 2H), 1.22 (d, J=6.8 Hz, 7H), 1.02 (d, J=6.9 Hz, 6H). ¹³C NMR (126 MHz, CDCl₃) δ 186.12 (d, J=18.4 Hz), 184.06 (d, J=18.5 Hz), 145.58, 135.96, 131.67 (d, J=6.8 Hz), 130.52, 128.09, 127.14, 126.51, 126.16, 126.15, 124.14, 123.38, 120.08, 114.86, 114.71 (d, J=6.9 Hz), 53.11, 28.37, 25.84, 25.60, 23.78, 23.53. HRMS calcd for C₂₉H₃₃AgN₃Cl⁺ (M+H⁺) 554.1487, found 554.1435.

### Synthesis of 2-(2,6-diisopropylphenyl)-9-(morpholin-4-yl)imidazo[1,5-a]quinolin-3-ylidene silver(I) chloride (60)

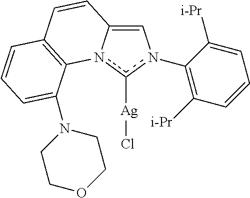

An oven-dried flask equipped with a stir bar was charged with the corresponding NHC·HCl salt (55) (135 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was obtained by trituration from hexanes as a grown solid (155 mg, 93%). ¹H NMR (500 MHz, Chloroform-d) δ 7.50 (dt, J=14.3, 7.8 Hz, 2H), 7.41 (ddd, J=11.1, 7.8, 1.6 Hz, 2H), 7.35-7.27 (m, 4H), 7.23 (d, J=9.4 Hz, 1H), 4.38 (td, J=11.6, 2.1 Hz, 2H), 3.88-3.76 (m, 2H), 3.27-3.15 (m, 2H), 2.84 (td, J=11.6, 3.1 Hz, 2H), 2.31 (p, J=6.8 Hz, 2H), 1.29 (d, J=6.9 Hz, 6H), 1.10 (d, J=6.8 Hz, 6H). ¹³C NMR (126 MHz, CDCl₃) δ 185.59 (d, J=18.5 Hz), 183.54 (d, J=18.5 Hz), 145.45, 144.27, 135.73, 131.86 (d, J=6.8 Hz), 130.68, 128.32, 127.41, 126.50, 126.48, 126.01, 125.99, 124.24, 124.13, 119.68, 115.14, 115.12, 114.94 (d, J=6.9 Hz), 66.55, 51.70, 28.43, 25.41, 23.59. HRMS caled for C₂₇H₃₁AgN₃O⁺ (M−Cl⁻) 520.1513, found 520.1540.

### Synthesis of 2-(2,4,6-trimethylphenyl)-9-(dimethylamino)imidazo[1,5-a]quinolin-3-ylidene silver(I) chloride (61)

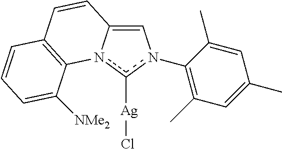

An oven-dried flask equipped with a stir bar was charged with the corresponding NHC·HCl salt (56) (110 mg, 0.3 mmol, 1.0 equiv), Ag₂O (83.4 mg, 0.36 mmol, 1.2 equiv), NaCl (35 mg, 0.6 mmol, 2.0 equiv), KCl (45 mg, 0.6 mmol, 2.0 equiv) and 4 Å MS (240 mg). The reaction mixture was placed under a positive pressure of argon and subjected to three evacuation/backfilling cycles under high vacuum. Anhydrous THE (4.0 mL) was added and the reaction mixture was stirred at 90° C. for 16 h. After the indicated time, the reaction mixture was diluted with CH₂Cl₂ (10 mL) and filtered. The solution was collected and concentrated. The title product was obtained by trituration from hexanes as a grown solid (121 mg, 85%). ¹H NMR (500 MHz, Chloroform-d) δ 7.44-7.37 (m, 2H), 7.33-7.24 (m, 3H), 7.17 (d, J=9.4 Hz, 1H), 7.00 (s, 2H), 2.75 (s, 6H), 2.36 (s, 3H), 2.02 (s, 6H). ¹³C NMR (126 MHz, CDCl₃) δ 185.47 (d, J=18.6 Hz), 183.40 (d, J=18.8 Hz), 145.37, 139.56, 136.66, 134.49, 132.12 (d, J=7.1 Hz), 129.54, 129.49, 128.00, 126.99, 126.30, 126.13, 126.11, 123.34, 120.21, 115.16, 115.14, 113.06 (d, J=6.8 Hz), 44.32, 21.15, 17.92. HRMS calcd for C₂₂H₂₃AgN₃⁺ (M−Cl⁻) 436.0937, found 436.0946.

### Synthesis of 2-(2,6-Diisopropylphenyl)-9-(dimethylamino)imidazo[1,5-a]quinolin-3-ylidene gold(I) chloride (62)

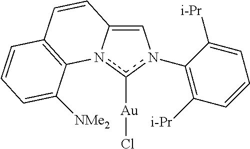

An oven-dried flask equipped with a stir bar was charged with the corresponding NHCAgCl salt (52) (154 mg, 0.3 mmol, 1.0 equiv), DCM (4 mL) and AuClSMe₂ (90 mg, 0.3 mmol, 1.0 equiv) was added. The mixture was stirred at room temperature for 16 hours. The solution was filtered and concentrated. The residue was purified by chromatography on silica gel to afford the title product (172 mg, 95%). ¹H NMR (500 MHz, Chloroform-d) δ 7.51 (t, J=7.8 Hz, 1H), 7.41 (t, J=7.8 Hz, 1H), 7.35-7.26 (m, 4H), 7.24 (d, J=9.1 Hz, 2H), 7.18 (d, J=9.4 Hz, 1H), 2.71 (s, 6H), 2.38 (p, J=6.9 Hz, 2H), 1.35 (d, J=6.8 Hz, 6H), 1.15 (d, J=6.9 Hz, 6H). ¹³C NMR (126 MHz, CDCl₃) δ 174.39, 145.48, 145.45, 135.57, 131.36, 130.54, 128.48, 127.25, 126.57, 124.62, 124.11, 121.87, 120.43, 114.86, 114.09, 42.76, 28.50, 24.65, 24.14. HRMS calcd for C₂₅H₃₀AuClN₃ (M+H⁺) 604.1788, found 604.1815.

### Synthesis of 2-(2,6-Diisopropylphenyl)-9-(diethylamino)imidazo[1,5-a]quinolin-3-ylidene gold(I) chloride (63)

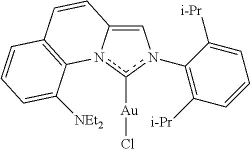

An oven-dried flask equipped with a stir bar was charged with the corresponding NHCAgCl salt (53) (163 mg, 0.3 mmol, 1.0 equiv), DCM (4 mL) and AuClSMe₂ (90 mg, 0.3 mmol, 1.0 equiv) was added. The mixture was stirred at room temperature for 16 hours. The solution was filtered and concentrated. The residue was purified by chromatography on silica gel to afford the title product (152 mg, 80%). ¹H NMR (500 MHz, Chloroform-d) δ 7.43 (t, J=7.8 Hz, 1H), 7.33 (t, J=7.8 Hz, 1H), 7.26-7.14 (m, 5H), 7.14-7.06 (m, 2H), 3.23 (dd, J=13.6, 6.9 Hz, 2H), 2.97 (dd, J=13.6, 7.0 Hz, 2H), 2.28 (p, J=6.8 Hz, 2H), 1.26 (d, J=6.9 Hz, 6H), 1.08-1.00 (m, 12H). ¹³C NMR (126 MHz, CDCl₃) δ 174.84, 145.45, 142.08, 135.64, 131.40, 130.55, 128.59, 126.73, 126.71, 126.68, 126.08, 124.11, 123.96, 122.29, 114.61, 114.59, 113.98, 45.53, 28.53, 24.67, 24.06, 11.03. HRMS caled for C₂₇H₃₃AuClN₃Na⁺ (M+Na⁺) 654.1921, found 654.1950.

### Synthesis of 2-(2,6-diisopropylphenyl)-9-(piperidin-1-yl)imidazo[1,5-a]quinolin-3-ylidene gold(I) chloride (64)

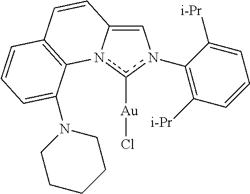

An oven-dried flask equipped with a stir bar was charged with the corresponding NHCAgCl salt (54) (167 mg, 0.3 mmol, 1.0 equiv), DCM (4 mL) and AuClSMe₂ (90 mg, 0.3 mmol, 1.0 equiv) was added. The mixture was stirred at room temperature for 16 hours. The solution was filtered and concentrated. The residue was purified by chromatography on silica gel to afford the title product (164 mg, 85%). ¹H NMR (500 MHz, Chloroform-d) δ 7.52 (t, J=7.8 Hz, 1H), 7.46-7.36 (m, 2H), 7.30 (t, J=6.1 Hz, 3H), 7.24 (dd, J=7.2, 1.7 Hz, 1H), 7.18 (q, J=9.4 Hz, 2H), 3.33 (d, J=11.8 Hz, 2H), 2.73 (d, J=12.0 Hz, 2H), 2.42 (p, J=6.9 Hz, 2H), 2.31 (q, J=11.7 Hz, 2H), 1.77 (dt, J=13.3, 4.4 Hz, 1H), 1.59 (d, J=12.5 Hz, 2H), 1.35 (d, J=6.8 Hz, 7H), 1.12 (d, J=6.8 Hz, 6H). ¹³C NMR (126 MHz, CDCl₃) δ 174.61, 145.93, 145.52, 135.54, 131.13, 130.53, 128.46, 127.39, 126.63, 124.71, 124.14, 121.86, 120.48, 114.50, 114.40, 52.25, 28.49, 25.41, 24.77, 24.24, 23.54. HRMS calcd for C₂₈H₃₄AuClN₃⁺ (M+H⁺) 644.2101, found 644.2109.

### Synthesis of 2-(2,6-diisopropylphenyl)-9-(morpholin-4-yl)imidazo[1,5-a]quinolin-3-ylidene gold(I) chloride (65)

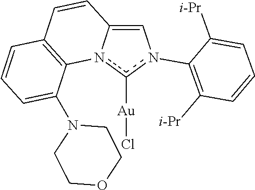

An oven-dried flask equipped with a stir bar was charged with the corresponding NHCAgCl salt (55) (167 mg, 0.3 mmol, 1.0 equiv), DCM (4 mL) and AuClSMe₂ (90 mg, 0.3 mmol, 1.0 equiv) was added. The mixture was stirred at room temperature for 16 hours. The solution was filtered and concentrated. The residue was purified by chromatography on silica gel to afford the title product (178 mg, 92%). ¹H NMR (500 MHz, Chloroform-d) δ 7.50 (dt, J=23.9, 7.8 Hz, 2H), 7.39-7.27 (m, 5H), 7.25-7.16 (m, 2H), 4.51 (t, J=11.2 Hz, 2H), 3.76 (d, J=11.4 Hz, 2H), 3.15 (d, J=11.6 Hz, 2H), 2.92 (t, J=11.4 Hz, 2H), 2.34 (p, J=6.9 Hz, 2H), 1.33 (d, J=6.9 Hz, 6H), 1.11 (d, J=6.9 Hz, 6H). ¹³C NMR (126 MHz, CDCl₃) δ 174.52, 145.38, 144.79, 135.36, 131.31, 130.66, 128.66, 127.69, 126.56, 124.87, 124.19, 123.12, 119.99, 114.92, 114.73, 65.67, 50.94, 28.56, 25.33, 23.52. HRMS calcd for C₂₇H₃₂AuClN₃O⁺ (M+H⁺) 646.1894, found 646.1911.

### Synthesis of 2-(2,4,6-trimethylphenyl)-9-(dimethylamino)imidazo[1,5-a]quinolin-3-ylidene gold(I) chloride (66)

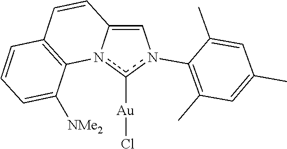

An oven-dried flask equipped with a stir bar was charged with the corresponding NHCAgCl salt (56) (142 mg, 0.3 mmol, 1.0 equiv), DCM (4 mL) and AuClSMe₂ (90 mg, 0.3 mmol, 1.0 equiv) was added. The mixture was stirred at room temperature for 16 hours. The solution was filtered and concentrated. The residue was purified by chromatography on silica gel to afford the title product (145 mg, 86%). ¹H NMR (500 MHz, Chloroform-d) δ 7.28 (t, J=7.9 Hz, 1H), 7.19 (d, J=3.5 Hz, 2H), 7.14-7.08 (m, 2H), 7.03 (d, J=9.4 Hz, 1H), 6.88 (s, 2H), 2.59 (s, 6H), 2.24 (s, 3H), 1.95 (s, 6H). ¹³C NMR (126 MHz, CDCl₃) δ 172.50, 144.35, 138.41, 135.23, 133.42, 130.53, 128.29, 127.37, 126.12, 125.17, 123.46, 120.70, 119.17, 113.92, 111.87, 41.74, 20.15, 17.03. HRMS calcd for C₂₂H₂₄AuClN₃⁺ (M+H⁺) 562.1324, found 562.1340.

### Example 6: Synthesis of Aryl Substituted Imidazo[1,5-a]quinoline N-Heterocyclic Carbene (NHC) Ligands

### Synthesis of 8-mesityl-2-methylquinoline (67)

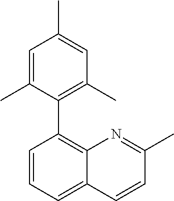

An oven-dried 100 mL round-bottomed flask equipped with a stir bar was charged with 8-bromo-2-methylquinoline (2.22 g, 10 mmol, 1.0 equiv), Ni(PCy₃)Cl₂ (138 mg, 0.2 mmol) and anhydrous THE (20 mL) under argon protection. Then 2-mesitylmagnesium bromide solution (15 mL, 1.0 M in THF, 15 mmol, 1.5 equiv) was added dropwise into the reaction at room temperature. The mixture was stirred at 95° C. for 24 hours. After the indicated time, the reaction was cooled down to room temperature and quenched by water. The mixture was diluted with EtOAc (50 mL), washed with H₂O (1×20 mL) and brine (1×20 mL). The organic layers were combined, dried, filtered, and concentrated. The residue was purified by chromatography on silica gel to afford the title product (2.48 g, 95%). ¹H NMR (500 MHz, Chloroform-d) δ 8.07 (d, J=8.4 Hz, 1H), 7.79 (dd, J=8.1, 1.6 Hz, 1H), 7.54 (dd, J=8.0, 7.0 Hz, 1H), 7.48 (dd, J=7.0, 1.7 Hz, 1H), 7.26 (d, J=8.3 Hz, 1H), 7.03 (s, 2H), 2.61 (s, 3H), 2.42 (s, 3H), 1.95 (s, 6H). ¹³C NMR (126 MHz, CDCl₃) δ 158.71, 146.30, 139.93, 137.31, 136.70, 136.21, 136.04, 130.62, 127.84, 126.93, 126.75, 125.27, 121.77, 25.80, 21.26, 20.83. HRMS calcd for C₁₉H₁₉NNa⁺ (M+Na⁺) 284.1410, found 284.1420.

### Synthesis of 8-mesitylquinoline-2-carbaldehyde (68)

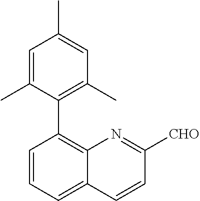 [SMILES: Cc1cc(C)c(-c2cccc3ccc(C=O)nc23)c(C)c1]

An oven-dried 100 mL round-bottomed flask equipped with a stir bar was charged with SeO₂ (1.33 g, 12 mmol, 1.2 equiv), 1,4-dioxane/H₂O (30 mL/0.3 mL). The mixture was stirred at 80° C. for 0.5 hour. Then 8-mesityl-2-methylquinoline (67) (10 mmol, 1.0 equiv) was added. The mixture was stirred at 80° C. for 3 hours. After the indicated time, the reaction was cooled down to room temperature and filtered, concentrated. The residue was purified by chromatography on silica gel to afford the title product (1.7 g, 62%). ¹H NMR (500 MHz, Chloroform-d) δ 9.97 (d, J=0.9 Hz, 1H), 8.35 (dd, J=8.4, 0.9 Hz, 1H), 8.02 (d, J=8.4 Hz, 1H), 7.92 (dd, J=8.2, 1.5 Hz, 1H), 7.74 (dd, J=8.2, 7.0 Hz, 1H), 7.62 (dd, J=7.0, 1.5 Hz, 1H), 7.02 (s, 2H), 2.41 (s, 3H), 1.90 (s, 6H). ¹³C NMR (126 MHz, CDCl₃) δ 194.57, 152.46, 146.30, 141.99, 137.56, 136.85, 136.49, 135.95, 131.70, 130.37, 129.03, 127.96, 127.22, 117.06, 21.20, 20.70. HRMS caled for C₃₈H₃₄N₂NaO₂⁺ (2M+Na⁺) 573.2512, found 573.2537.

### Synthesis of 2-(2,6-Diisopropylphenyl)-9-(2,4,6-trimethylphenyl)imidazo[1,5-a]quinolin-2-ium chloride (69)

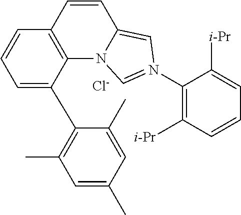

An oven-dried 100 mL round-bottomed flask equipped with a stir bar was charged with 8-mesitylquinoline-2-carbaldehyde (68) (10 mmol, 1.0 equiv), 2,6-diisopropylaniline (10 mmol, 1.0 equiv), paraformaldehyde (450 mg, 15 mmol, 1.5 equiv) and toluene (25 mL). HCl (4.0 M in dioxane, 5.0 mL, 2.0 equiv) was added dropwise into the reaction at room temperature. The mixture was stirred at 110° C. for 24 hours. After the indicated time, the reaction was cooled down to room temperature and concentrated. The residue was purified by chromatography on silica gel (CH₂Cl₂/MeOH=20/1) to afford the title product (3.7 g, 76%). ¹H NMR (500 MHz, Chloroform-d) δ 8.32 (s, 1H), 8.04 (d, J=9.6 Hz, 1H), 7.99 (dd, J=7.9, 1.6 Hz, 1H), 7.84 (t, J=7.6 Hz, 1H), 7.74 (d, J=9.2 Hz, 1H), 7.55 (t, J=7.8 Hz, 1H), 7.49 (dd, J=7.4, 1.6 Hz, 1H), 7.27 (d, J=8.0 Hz, 2H), 6.99 (s, 2H), 2.24 (s, 3H), 1.96 (p, J=6.8 Hz, 2H), 1.86 (s, 6H), 1.15 (d, J=6.7 Hz, 6H), 0.93 (d, J=6.8 Hz, 6H). ¹³C NMR (126 MHz, CDCl₃) δ 144.82, 140.44, 135.83, 133.84, 132.55, 132.27, 131.62, 131.01, 130.06, 129.98, 129.90, 128.75, 127.04, 126.97, 126.83, 124.60, 118.27, 116.45, 28.61, 24.65, 23.10, 21.08, 20.21. HRMS calcd for C₃₂H₃₅N₂⁺ (M−Cl⁻) 447.2795, found 447.2833.

### Example 7: Synthesis of Aryl Substituted Imidazo[1,5-a]quinoline NHC Ligand-Metal Complexes

### Synthesis of 2-(2,6-Diisopropylphenyl)-9-(2,4,6-trimethylphenyl)imidazo[1,5-a]quinolin-3-ylidene gold(I) chloride (70)

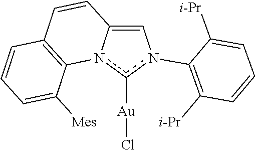

An oven-dried flask equipped with a stir bar was charged with the corresponding NHC·HCl salt (69) (145 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 mL, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was was purified by chromatography on silica gel to afford the title product (112 mg, 55%). ¹H NMR (500 MHz, Chloroform-d) δ 7.64 (dd, J=7.6, 1.6 Hz, 1H), 7.56 (t, J=7.6 Hz, 1H), 7.42 (ddd, J=7.8, 4.9, 3.2 Hz, 2H), 7.30-7.26 (m, 2H), 7.23 (d, J=9.3 Hz, 1H), 7.19 (d, J=7.8 Hz, 2H), 6.92 (s, 2H), 2.29-2.22 (m, 5H), 1.97 (s, 6H), 1.18 (d, J=6.8 Hz, 6H), 1.08 (d, J=6.9 Hz, 6H). ¹³C NMR (126 MHz, CDCl₃) δ 145.12, 137.44, 135.48, 134.99, 130.53, 130.30, 127.86, 127.49, 126.68, 126.50, 124.09, 114.74, 114.60, 28.45, 24.80, 23.81, 21.49. HRMS calcd for C₃₂H₃₄AuClN₂Na⁺ (M+Na⁺) 701.1968, found 701.1987.

### Example 8: Synthesis of Control NHC Ligands and Ligand-Metal Complexes

### Synthesis of 2-(2,6-Diisopropylphenyl)-5-isopropylimidazo[1,5-a]pyridin-2-ium chloride (A2)

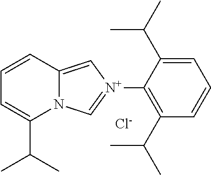 [SMILES: CC(C)c1cccc(C(C)C)c1-[n+]1cc2cccc(C(C)C)n2c1 ; [Cl-]]

An oven-dried 100 mL round-bottomed flask equipped with a stir bar was charged with 2-(1,3-dioxolan-2-yl)-6-isopropylpyridine (1.93 g, 10 mmol, 1.0 equiv), 2,6-diisopropylaniline (1.77 g, 10 mmol, 1.0 equiv), paraformaldehyde (450 mg, 15 mmol, 1.5 equiv) and EtOH (25 mL). HCl (4.0 M in dioxane, 5.0 mL, 2.0 equiv) was added dropwise into the reaction at room temperature. The mixture was stirred at 70° C. for 24 hours. After the indicated time, the reaction was cooled down to room temperature and concentrated. The residue was purified by chromatography on silica gel (CH₂Cl₂/MeOH=20/1) to afford the title product (2.14 g, 60%). ¹H NMR (500 MHz, CDCl₃) δ 11.68 (s, 1H), 7.71 (d, J=9.3 Hz, 1H), 7.69 (s, 1H), 7.57 (t, J=7.8 Hz, 1H), 7.36 (d, J=7.8 Hz, 2H), 7.34-7.31 (m, 1H), 7.00 (d, J=7.0 Hz, 1H), 4.49 (s, 1H), 2.23-2.15 (m, 2H), 1.49 (d, J=4.3 Hz, 6H), 1.34 (d, J=6.6 Hz, 6H), 1.17 (d, J=6.7 Hz, 6H). ¹³C NMR (126 MHz, CDCl₃) δ 145.17, 145.03, 131.94, 130.89, 130.75, 129.31, 126.37, 124.56, 123.36, 115.41, 114.58, 112.88, 29.51, 28.89, 24.52, 24.39, 20.28. HRMS calcd for C₂₂H₂₉N₂⁺ (M−Cl⁻) 321.2325, found 321.2331.

### Synthesis of 2-(2,6-Diisopropylphenyl)-5-isopropylimidazo[1,5-a]pyridin-3-ylidene gold(I) chloride (A3)

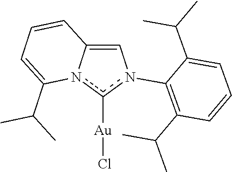

An oven-dried flask equipped with a stir bar was charged with the corresponding NHC·HCl salt (A2) (107 mg, 0.3 mmol, 1.0 equiv), AuClSMe₂ (90 mg, 0.3 mmol, 1.0 equiv) and finely powdered K₂CO₃ (83 mg, 0.6 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (104 mg, 63%). ¹H NMR (500 MHz, CDCl₃) δ 7.44 (t, J=7.8 Hz, 1H), 7.30-7.17 (m, 4H), 6.93 (dd, J=9.0, 7.1 Hz, 1H), 6.60 (d, J=6.8 Hz, 1H), 5.24 (dq, J=13.3, 6.6 Hz, 1H), 2.31-1.93 (m, 2H), 1.40 (d, J=6.7 Hz, 6H), 1.25 (d, J=6.8 Hz, 6H), 1.04 (d, J=6.9 Hz, 6H). ¹³C NMR (126 MHz, CDCl₃) δ 163.16, 147.34, 145.23, 135.77, 132.04, 130.75, 124.19, 123.48, 115.72, 113.67, 110.55, 30.37, 28.47, 24.58, 24.26, 22.14. HRMS calcd for C₂₂H₂₈AuClN₂Na⁺ (M⁺+Na) 575.1499, found 575.1506. *The compound is ineffective in promoting Au(I)/(III) reactivity.

### Synthesis of (E)-2-(((2,6-Diisopropylphenyl)imino)methyl)-N,N-dimethylpyridin-4-amine

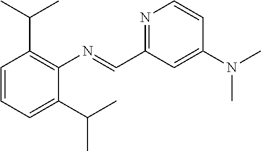 [SMILES: CC(C)c1cccc(C(C)C)c1/N=C/c1cc(N(C)C)ccn1]

An oven-dried 20 mL screw cap vial equipped with a magnetic stir bar was charged with 4-(dimethylamino)picolinaldehyde (750.9 mg, 5 mmol, 1.0 equiv), 2,6-diisopropylaniline (886.5 mg, 5 mmol, 1.0 equiv), Na₂SO₄ (1.42 g, 10 mmol, 2.0 equiv), formic acid (46.0 mg, 1 mmol, 2.0 equiv) and 15 mL anhydrous MeOH. The reaction mixture was stirred at room temperature for 24 h. After the indicated time, the reaction was concentrated. The residue was purified by chromatography on silica gel to afford the title product (866.5 mg, 56%). ¹H NMR (500 MHz, CDCl₃) δ 8.35 (d, J=5.9 Hz, 1H), 8.24 (s, 1H), 7.52 (d, J=2.6 Hz, 1H), 7.13 (dt, J=8.6, 6.8 Hz, 3H), 6.60 (dd, J=5.9, 2.7 Hz, 1H), 3.07 (s, 6H), 3.00 (dd, J=13.7, 6.9 Hz, 2H), 1.19 (d, J=7.0 Hz, 12H). ¹³C NMR (126 MHz, CDCl₃) δ 164.06, 154.82, 154.28, 149.78, 148.59, 137.38, 124.33, 123.04, 108.34, 103.52, 39.29, 27.93, 23.58. HRMS calcd for C₂₀H₂₈N₃⁺ (M⁺+H) 310.2278, found 310.2280.

### Synthesis of N-(2,6-Diisopropylphenyl)-N-((4-(dimethylamino)pyridin-2-yl)methyl)formamide

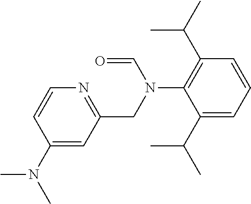 [SMILES: CC(C)c1cccc(C(C)C)c1N(C=O)Cc1cc(N(C)C)ccn1]

An oven-dried 50 mL screw cap vial equipped with a magnetic stir bar was charged with (E)-2-(((2,6-diisopropylphenyl)imino)methyl)-N,N-dimethylpyridin-4-amine (618.9 mg, 2.0 mmol, 1.0 equiv), paraformaldehyde (78.0 mg, 2.6 mmol, 1.3 equiv) and toluene (15 mL). TMSCl (434.5 mg, 4.0 mmol, 2.0 equiv) was added dropwise at room temperature and the reaction mixture was stirred at 80° C. for 12 hours. After the indicated time, the reaction was cooled down to room temperature and concentrated. The residue was purified by chromatography on silica gel to afford the title product (237.6 mg, 35%). Isomer A: ¹H NMR (500 MHz, CDCl₃) δ 8.77 (s, 1H), 8.23 (d, J=6.0 Hz, 1H), 7.31 (dd, J=16.0, 8.2 Hz, 1H), 7.15 (d, J=7.7 Hz, 2H), 6.43-6.37 (m, 1H), 6.08 (d, J=2.5 Hz, 1H), 4.56 (s, 2H), 2.89 (s, 6H), 2.83 (dq, J=14.5, 7.1 Hz, 2H), 1.12 (d, J=6.8 Hz, 6H), 0.95 (d, J=6.8 Hz, 6H). Isomer B: ¹H NMR (500 MHz, CDCl₃) δ 8.15 (s, 1H), 8.06 (d, J=6.0 Hz, 1H), 7.31 (dd, J=16.0, 8.2 Hz, 1H), 7.15 (d, J=7.7 Hz, 2H), 6.79 (d, J=2.5 Hz, 1H), 6.43-6.37 (m, 1H), 4.80 (s, 2H), 2.99 (s, 6H), 2.83 (dq, J=14.5, 7.1 Hz, 2H), 1.09 (d, J=6.9 Hz, 6H), 1.00 (d, J=6.8 Hz, 6H). ¹³C NMR (isomers A and B) (126 MHz, CDCl₃) δ 163.55, 155.94, 155.01, 154.77, 154.69, 149.82, 148.82, 147.66, 146.61, 135.72, 134.17, 129.31, 128.77, 124.31, 124.10, 107.33, 106.72, 105.89, 105.83, 57.74, 53.86, 39.19, 39.07, 28.69, 28.40, 25.43, 24.94, 23.53, 23.25. HRMS caled for C₂₁H₃₀N₃O⁺ (M++H) 340.2383, found 340.2387.

### Synthesis of 2-(2,6-Diisopropylphenyl)-7-(dimethylamino)imidazo[1,5-a]pyridin-2-ium chloride (A4)

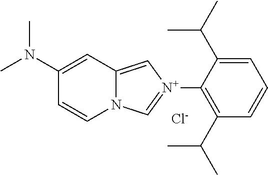 [SMILES: CC(C)c1cccc(C(C)C)c1-[n+]1cc2cc(N(C)C)ccn2c1 ; [Cl-]]

An oven-dried 10 mL screw cap vial equipped with a magnetic stir bar was charged with N-(2,6-diisopropylphenyl)-N-((4-(dimethylamino)pyridin-2-yl)methyl)formamide (169.7 mg, 0.5 mmol, 1.0 equiv) and toluene (5 mL). Phosphoryl chloride (84.3 mg, 0.55 mmol, 1.1 equiv) was added dropwise at room temperature and the reaction mixture was stirred at 80° C. for 12 hours. After the indicated time, the reaction was cooled down to room temperature and concentrated. The residue was purified by chromatography on silica gel to afford the title product (75.2 mg, 42%)⁹. ¹H NMR (500 MHz, CDCl₃) δ 9.88 (s, 1H), 9.01 (d, J=7.8 Hz, 1H), 7.52 (t, J=7.8 Hz, 1H), 7.29 (d, J=7.8 Hz, 2H), 7.05 (s, 1H), 7.00-6.89 (m, 1H), 6.34 (s, 1H), 3.08 (s, 6H), 2.19 (dt, J=13.5, 6.8 Hz, 2H), 1.17 (d, J=6.8 Hz, 6H), 1.13 (d, J=6.8 Hz, 6H). ¹³C NMR (126 MHz, CDCl₃) δ 146.60, 145.17, 133.38, 131.70, 130.87, 125.87, 125.58, 124.45, 111.39, 108.42, 88.34, 40.14, 28.56, 24.51, 24.44. HRMS calcd for C₂₁H₂₈N₃⁺ (M−Cl⁻) 322.2278, found 322.2283.

### Synthesis of 2-(2,6-Diisopropylphenyl)-7-(dimethylamino)imidazo[1,5-a]pyridin-3-ylidene gold(I) chloride (A5)

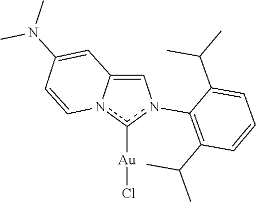

An oven-dried flask equipped with a stir bar was charged with the corresponding NHC·HCl salt (A4) (71.6 mg, 0.2 mmol, 1.0 equiv), AuClSMe₂ (58.9 mg, 0.2 mmol, 1.0 equiv) and finely powdered K₂CO₃ (55.2 mg, 0.4 mmol, 2.0 equiv). The reaction mixture was placed under a positive pressure of argon and subjected to three evacuation/backfilling cycles under high vacuum. Acetone (6.0 ml, 0.05 M) was added and the reaction mixture was stirred at 60° C. for 16 h. After the indicated time, the reaction mixture was diluted with CH₂Cl₂ (10 mL) and filtered. The solution was collected and concentrated. The title product was obtained by trituration from hexanes as a white solid (80.8 mg, 73%). ¹H NMR (500 MHz, CDCl₃) δ 8.44 (d, J=8.0 Hz, 1H), 7.48 (t, J=7.8 Hz, 1H), 7.26 (d, J=7.9 Hz, 2H), 6.79 (s, 1H), 6.65 (dd, J=8.0, 2.4 Hz, 1H), 6.14 (s, 1H), 3.04 (s, 6H), 2.30 (dt, J=13.6, 6.8 Hz, 2H), 1.28 (d, J=6.8 Hz, 6H), 1.11 (d, J=6.9 Hz, 6H). ¹³C NMR (126 MHz, CDCl₃) δ 162.03, 145.81, 145.49, 134.77, 132.37, 130.42, 128.13, 124.06, 108.38, 108.28, 89.15, 40.22, 28.31, 24.63, 24.50. HRMS calcd for C₂₁H₂₇AuClN₃Na+ (M⁺+Na) 576.1451, found 576.1457. *The compound is ineffective in promoting Au(I)/(III) reactivity.

### Synthesis of (2-(2,6-Diisopropylphenyl)-5-(dimethylamino)imidazo[1,5-a]pyridin-3-ylidene)(2,2′-biphenyl) gold(III) chloride (A6)

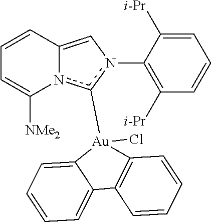

To a mixture of the corresponding Au(NHC)Cl (16) (55.4 mg, 0.1 mmol, 1.0 equiv) and AgSbF₆ (38 mg, 0.11 mmol, 1.0 equiv) was added CH₂Cl₂ (5 mL) at room temperature and the resulting mixture was stirred for 15 min. After the indicated time, the reaction mixture was passed through glass fiber to remove AgCl, followed by addition of biphenylene (34.2 mg, 0.225 mmol, 2.25 equiv). After stirring at room temperature for 3 h, tetrabutylammonium chloride (34.7 mg, 0.125 mmol, 1.25 equiv) was added and the stirring was continued for 3 h. The title product was purified by chromatography on silica gel as a yellow solid (55 mg, 78%). ¹H NMR (500 MHz, CDCl₃) δ 8.09 (dd, J=7.6, 1.0 Hz, 1H), 7.44 (s, 1H), 7.41 (t, J=7.8 Hz, 1H), 7.31 (ddd, J=9.3, 6.5, 2.9 Hz, 4H), 7.10 (ddd, J=16.3, 7.7, 1.2 Hz, 2H), 7.05-6.96 (m, 3H), 6.58 (td, J=7.6, 1.4 Hz, 1H), 6.34 (d, J=6.3 Hz, 1H), 6.10 (d, J=7.2 Hz, 1H), 2.87 (s, 3H), 2.54 (dtd, J=34.2, 13.5, 6.7 Hz, 5H), 1.47 (d, J=6.6 Hz, 3H), 1.12 (d, J=6.8 Hz, 3H), 0.91 (d, J=6.9 Hz, 3H), 0.60 (d, J=6.7 Hz, 3H). ¹³C NMR (126 MHz, CDCl₃) δ 175.44, 158.27, 153.87, 153.57, 153.33, 149.40, 146.58, 144.92, 134.82, 133.20, 132.92, 131.79, 130.68, 126.61, 126.38, 126.23, 126.19, 124.39, 124.04, 123.99, 121.08, 120.21, 117.09, 113.42, 104.77, 28.98, 28.32, 27.03, 26.05, 22.83, 21.86. HRMS calcd for C₃₃H₃₅AuClN₃Na⁺ (M⁺+Na) 728.2077, found 728.2080. *The x-ray structure shows stabilizing effect of the C₅ amino group on Au(III).

### Example 9: Characterization of NHC Ligands and Ligand-Metal Complexes

### Electronic Characterization

NHC—Se (1 complex) (A1) and NHC—Rh(I) complexes (38-40) were synthesized and characterized with regard to π-acceptance and σ-donation, respectively. The δ_{Se} value of 214.65 ppm for (A1) (CDCl₃) can be compared with IPr (δ_{Se}=90 ppm), indicating much stronger π-accepting properties of this class of ligands. The avg. ν_{CO}=2063.9 cm⁻¹ for cationic 40 can be compared with cationic bis-NHC complex avg. ν_{CO}=2057 cm⁻¹ (Canac, JACS 2008, 130, 8406), indicating strong σ-donation of this class of ligands. One-bond CH J-values for coupling constants from ¹³C satellites of the ¹H NMR spectrum (NHC salt) provide good indication of σ-donating properties of an NHC ligand. The value of 227.7 Hz for ligand (1) (CDCl₃) is consistent with this ligand being strongly σ-donating (cf., IPr: ¹J_{CH}=223.7 Hz).

### Complexation Studies

The complexation studies demonstrate that the ligands of the present disclosure are useful for the synthesis of well-defined, air and/or moisture stable complexes with Au(I), Ag(I), Cu(I), Pd(II), Rh(I) and Se. In certain embodiments, the cationic complexes are formed due to amine-to-metal coordination, and the complexes remain stable in a solid state, permitting their application in cationic catalysis.

### X-Ray Structural Analysis

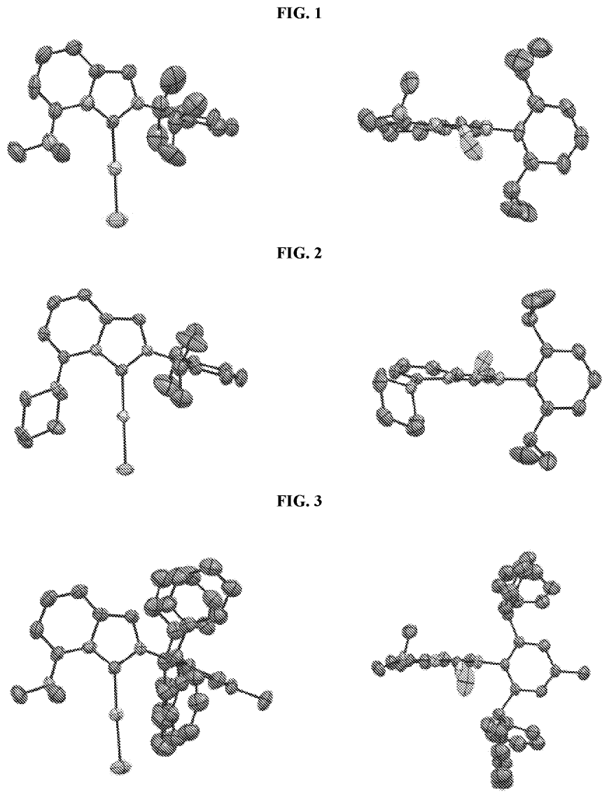

The structures of several Au(I) complexes (FIGS. 1-3) and representative Ag(I), Cu(I), and Pd(II) complexes have been fully characterized by x-ray analysis.

The % buried volume (% V_{bur}) of 16 is 42.8%, which can be compared with (% V_{bur}) of 43.2% determined for 20; (% V_{bur}) of 47.8% determined for 18; (% V_{bur}) of 40.9% for 23 and (% V_{bur}) of 44.9% determined for 22. These values can be compared with (% V_{bur}) of 35.5% isoelectronic sterically-unbiased ligand (A4), which may indicate that the presence of the amino substituent in the C₅ position leads to a significant increase of steric volume (cf 16 of 42.8%, Δ of 7.3%)

This steric increase effect can be compared with steric impact from 1,3-Bis(2,4,6-trimethylphenyl)-1,3-dihydro-2H-imidazol-2-ylidene (IMes): % V_{bur}=36.5% to 1,3-bis(2,6-diisopropylphenyl)imidazol-2-ylidene (IPr): % V_{bur}=45.4% (Δ of 8.9%). Furthermore, there is a significant variation of steric impact within the series of NHC ligands disclosed herein which is enforced by the N-wingtip substituent (40.9% to 47.8%, Δ of 6.9%).

Without wishing to be bound by theory, these values indicate that this class of ligands (1) is characterized by the steric effect in the range of imidazolylidene IMes/IPr, which has been determined as ideal for catalysis, and (2) enables the variation of the size of catalytic pocket, which is an important factor in tuning reactivity to a specific class of substrates and reactions. Furthermore, there is coplanarity of the hard N atom and transition-metals coordinated to the NHC center (e.g. [16, Me₂N—Au distance of 3.126 Å; [18, Me₂N—Au of 3.144 Å; [22, PipN-Au of 3.114 Å), which is unavailable in other classes of NHC ligands developed to date.

### Example 10: Catalysis

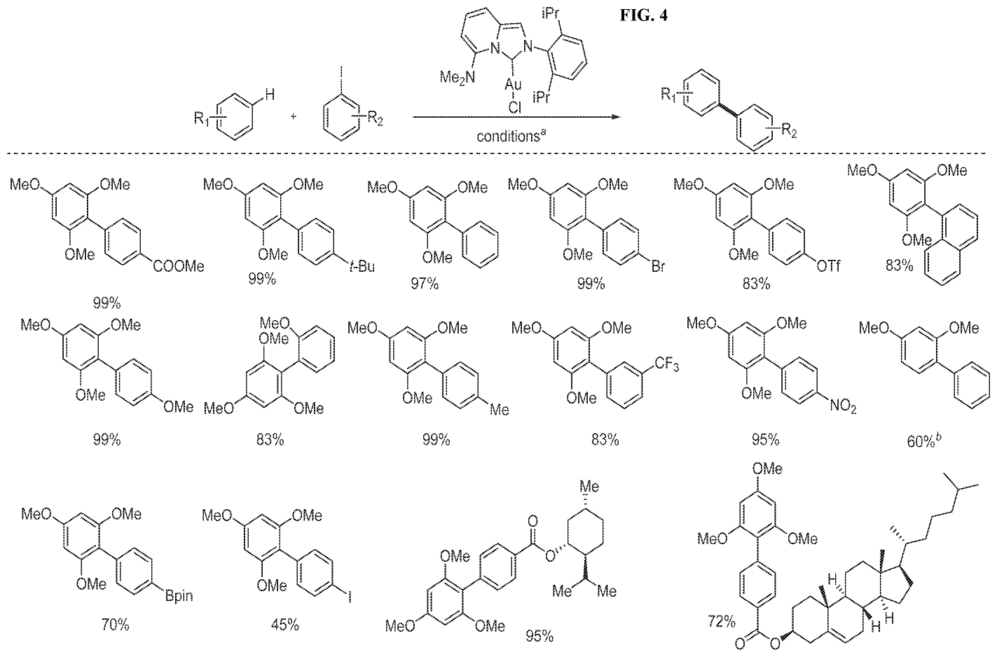

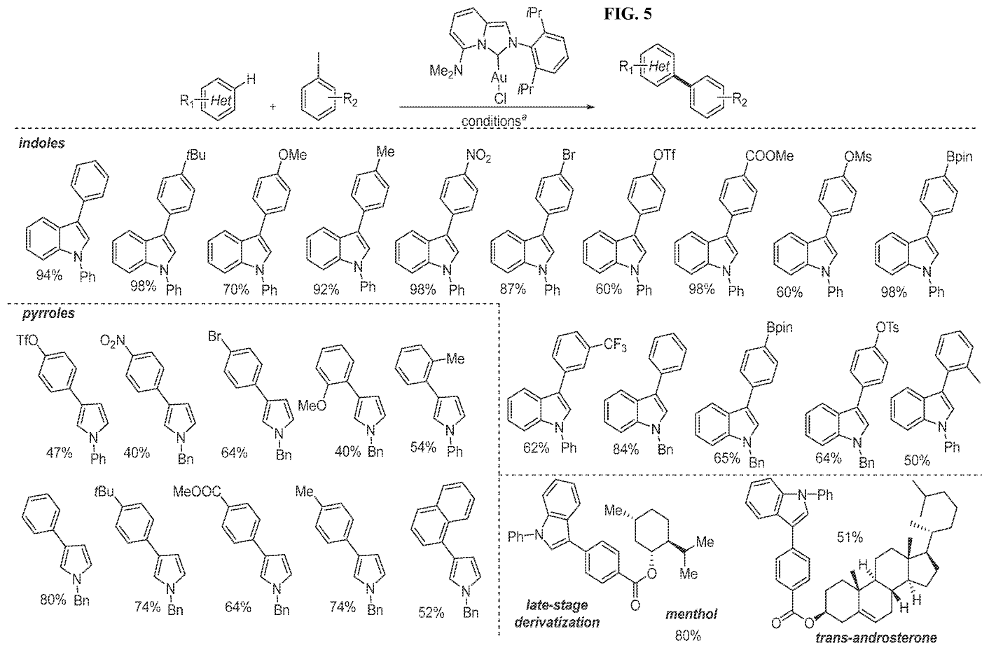

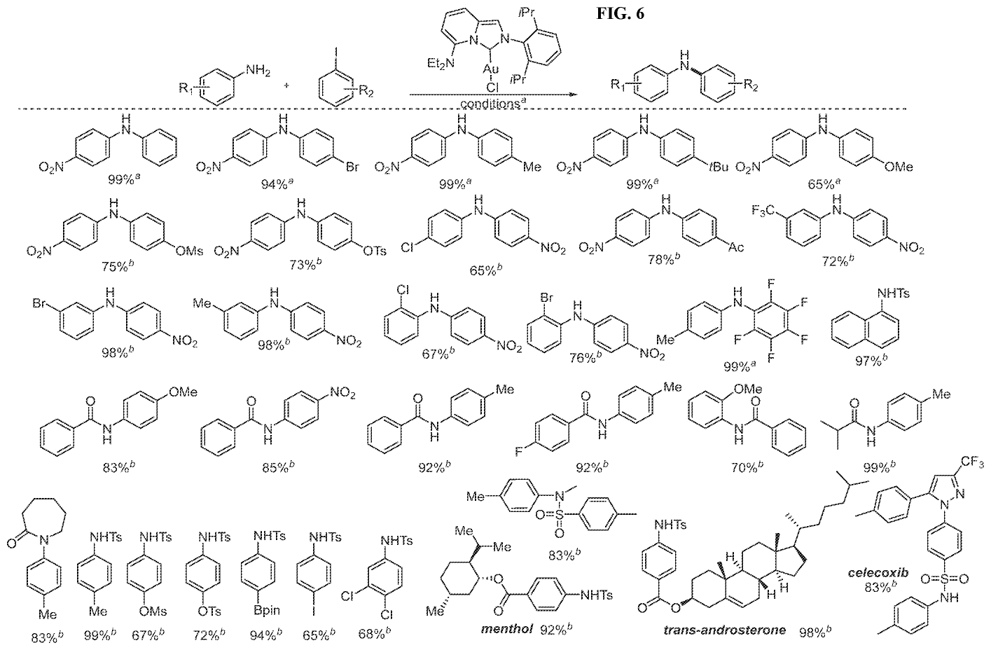

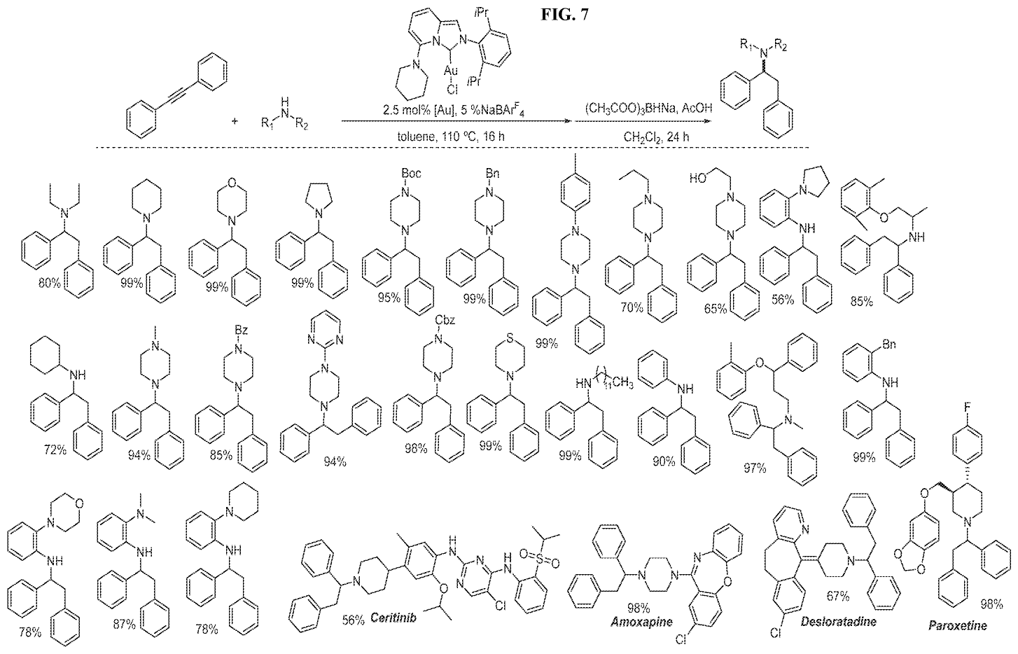

Catalytic studies were performed to evaluate the catalytic activity of certain compounds of the present disclosure. Four non-limiting classes of reactions have been developed, including: (1) Oxidative Au(I)/Au(III)C—C coupling by C—H arylation (FIG. 4); (2) Oxidative Au(I)/Au(III)C—C coupling by C—H heteroarylation (FIG. 5); (3) Oxidative Au(I)/Au(III)C—N coupling by C—X amination (FIG. 6); and (4) Electrophilic Au(I) hydroamination of internal alkynes with aliphatic and aromatic amines (FIG. 7).

Feasibility studies (Au catalysis) have been conducted demonstrating the following reactions: (1) Electrophilic Au(I) hydroamination of internal alkynes with parent hydrazine; (2) Electrophilic Au(I) hydroamination of terminal alkynes with aromatic amines; (3) Electrophilic Au(I) hydration of internal alkynes. Feasibility studies (Pd(0)/Pd(II) catalysis) have been conducted demonstrating the following reactions: (1) amination of Ar—Cl; (2) Kumada cross-coupling of Ar—Cl; (3) arylation of ArC(O)—Cl; and (4) aminocarbonylation of Ar—I.

The feasibility studies represent unoptimized results for the use of the compounds of the present disclosure in Au and Pd-catalysis. Nonetheless, the utility of the compounds disclosed herein has been demonstrated in a number of catalytic transformations.

### General Procedure for Oxidative Au(I)/Au(III)C—C Coupling by C—H Arylation (General Procedure I)

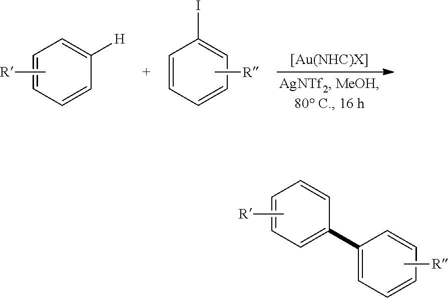

An oven-dried vial equipped with a stir bar was charged with iodoarene (1.0 equiv), arene (1.0 equiv), [Au(NHC)X] (5 mol %), AgNTf₂ (1.1 equiv), MeOH (0.125 M) at room temperature. The reaction mixture was placed in a preheated oil bath at 80° C., and stirred for 16 hours at 80° C. After the indicated time, the reaction mixture was cooled down to room temperature, diluted with CH₂Cl₂ (10 mL), filtered, and concentrated. The residue was analyzed by ¹H NMR (CDCl₃, 500 MHz) and GC-MS using internal standard. Purification by chromatograpFThy on silica gel (EtOAc/hexanes or CH₂Cl₂/MeOH) afforded the title product. X, R′, and R″ are defined within the scope of the present disclosure.

### Synthesis of methyl 2′,4′,6′-trimethoxy-[1,1′-biphenyl]-4-carboxylate (General Procedure I)

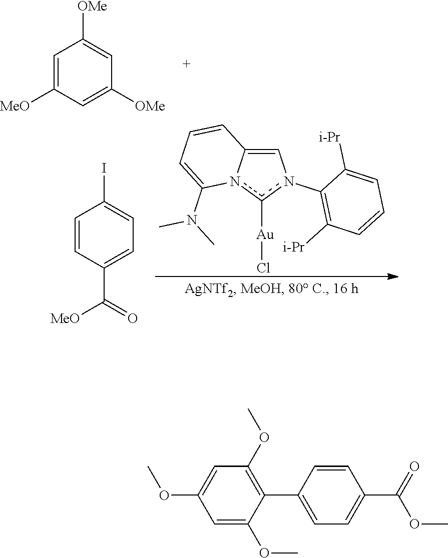

According to General Procedure (I), the reaction of methyl 4-iodobenzoate (0.2 mmol, 1.0 equiv), 1,3,5-trimethoxybenzene (0.20 mmol, 1.0 equiv), 16 (0.01 mmol, 5 mol %), AgNTf₂ (0.22 mmol, 1.1 equiv), MeOH (0.125 M) for 16 hours at 80° C., afforded the title product in 99% yield (60.0 mg). ¹H NMR (500 MHz, CDCl₃) δ 8.06 (d, J=8.3 Hz, 2H), 7.43 (d, J=8.3 Hz, 2H), 6.24 (s, 2H), 3.93 (s, 3H), 3.88 (s, 3H), 3.73 (s, 6H). ¹³C NMR (126 MHz, CDCl₃) δ 167.30, 161.04, 158.24, 139.46, 131.37, 128.88, 128.02, 111.42, 90.91, 55.86, 55.42, 51.98. Spectroscopic data matched literature values.

### General Procedure for Oxidative Au(I)/Au(III)C—C Coupling by C—H Heteroarylation (General Procedure II)

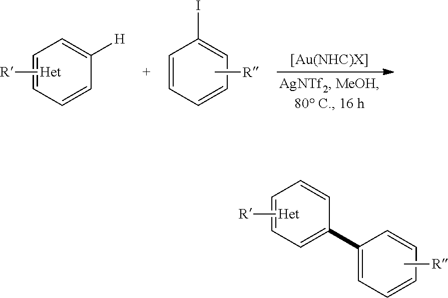

An oven-dried vial equipped with a stir bar was charged with iodoarene (1.0 equiv), heteroarene (2.0 equiv), [Au(NHC)Cl] (5 mol %), AgNTf₂ (1.1 equiv), MeOH (0.125 M) at room temperature. The reaction mixture was placed in a preheated oil bath at 80° C., and stirred for 16 hours at 80° C. After the indicated time, the reaction mixture was cooled down to room temperature, diluted with CH₂Cl₂ (10 mL), filtered, and concentrated. The residue was analyzed by ¹H NMR (CDCl₃, 500 MHz) and GC-MS using internal standard. Purification by chromatography on silica gel (EtOAc/hexanes) afforded the title product. Note: “Het” indicates a heteroaryl compound and/or moiety, and is not limited to 6-membered heteroaryl species. X, R′, and R″ are defined within the scope of the present disclosure.

### Synthesis of 1,3-diphenyl-1H-indole

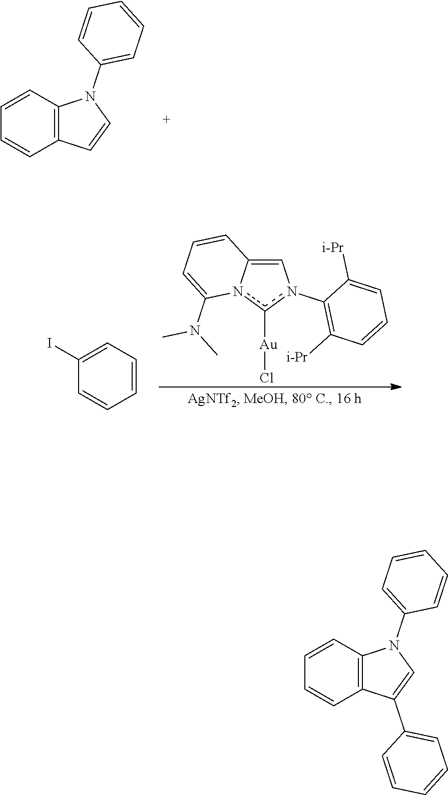

According to General Procedure II, the reaction of iodobenzene (0.2 mmol, 1.0 equiv), 1-phenyl-1H-indole (0.40 mmol, 2.0 equiv), 16 (0.01 mmol, 5 mol %), AgNTf₂ (0.22 mmol, 1.1 equiv), MeOH (0.125 M) for 16 hours at 80° C., afforded the title product in 94% yield (50.6 mg). ¹H NMR (500 MHz, CDCl₃) δ 8.02-7.98 (m, 1H), 7.75-7.70 (m, 2H), 7.61 (d, J=7.7 Hz, 1H), 7.54 (dt, J=14.0, 6.8 Hz, 5H), 7.48 (t, J=7.7 Hz, 2H), 7.41-7.36 (m, 1H), 7.32 (t, J=7.4 Hz, 1H), 7.30-7.24 (m, 2H). ¹³C NMR (126 MHz, CDCl₃) δ 139.56, 136.69, 135.14, 129.72, 128.86, 127.64, 127.17, 126.70, 126.25, 125.56, 124.52, 122.83, 120.91, 120.17, 119.15, 110.86. Spectroscopic data matched literature values.

### General Procedure for Oxidative Au(I)/Au(III)C—N Coupling by C—X Amination (General Procedure III

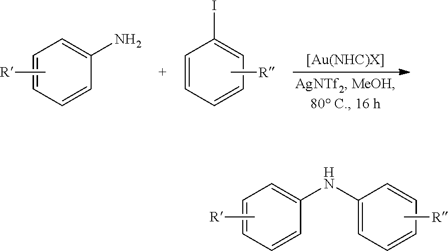

An oven-dried vial equipped with a stir bar was charged with iodoarene (1.0 equiv), aniline (1.0 equiv), [Au(NHC)Cl] (2.5-5 mol %), AgNTf₂ (1.1 equiv), MeOH (0.125 M) at room temperature. The reaction mixture was placed in a preheated oil bath at 80° C., and stirred for 16 hours at 80° C. After the indicated time, the reaction mixture was cooled down to room temperature, diluted with CH₂Cl₂ (10 mL), filtered, and concentrated. The residue was analyzed by ¹H NMR (CDCl₃, 500 MHz) and GC-MS using internal standard. Purification by chromatography on silica gel (EtOAc/hexanes or CH₂Cl₂/MeOH) afforded the title product. X, R′ and R″ are defined within the scope of the present disclosure.

### Synthesis of 4-Nitro-N-phenylaniline

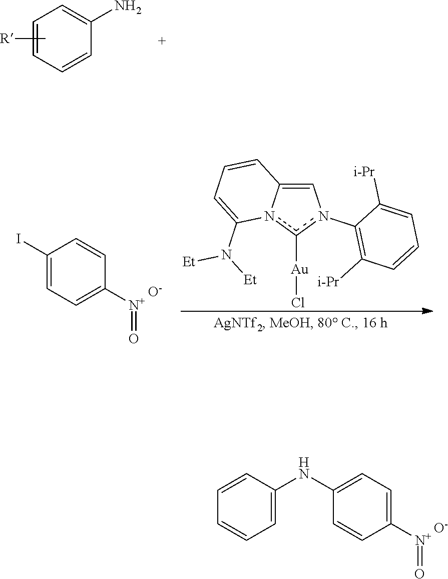

According to General Procedure III, the reaction of 1-iodo-4-nitrobenzene (0.2 mmol, 1.0 equiv), aniline (0.20 mmol, 1.0 equiv), 19 (0.005 mmol, 2.5 mol %), AgNTf₂ (0.22 mmol, 1.1 equiv), MeOH (0.125 M) for 16 hours at 80° C., afforded the title product in 99% yield (42.4 mg). ¹H NMR (500 MHz, CDCl₃) δ 8.15-8.07 (m, 2H), 7.39 (t, J=7.9 Hz, 2H), 7.21 (d, J=7.6 Hz, 2H), 7.17 (t, J=7.4 Hz, 1H), 6.97-6.82 (m, 2H), 6.28 (s, 1H). ¹³C NMR (126 MHz, CDCl₃) δ 150.17, 139.83, 139.50, 129.77, 126.26, 124.71, 121.96, 113.71. Spectroscopic data matched literature values.

### General Procedure for Electrophilic Au(I) Hydroamination of Internal Alkynes with Aliphatic and Aromatic Amines (General Procedure IV)

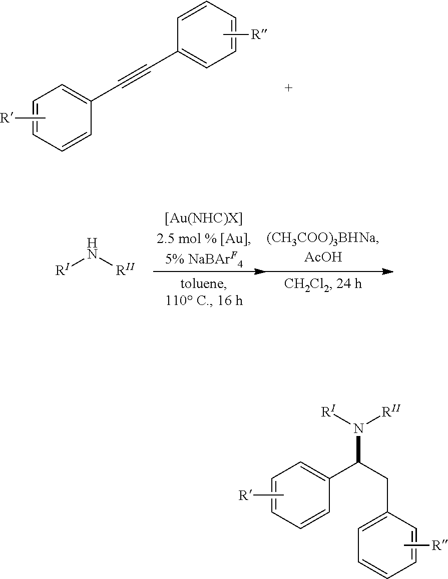

An oven-dried vial equipped with a stir bar was charged with alkyne (1.0 equiv), amine (1.2 equiv), [Au(NHC)Cl] (2.5 mol %), NaBAr^{F} (5.0 mol %), toluene (0.5 M) under argon at room temperature. The reaction mixture was placed in a preheated oil bath at 110° C. and stirred for 16 hours. After the indicated time, the reaction mixture was cooled down to room temperature and concentrated. Sodium triacetoxyborohydride (2.0 equiv), acetic acid (2.0 equiv) and CH₂Cl₂ (0.1 M) were added and the reaction mixture was stirred for 24 h at room temperature. After the indicated time, the reaction was quenched with NaOH (1.0 M, aq) and extracted with CH₂Cl₂ (15 ml). The combined organic phases were dried, filtered and concentrated. The residue was analyzed by ¹H NMR (CDCl₃, 500 MHz) and GC-MS using internal standard. Purification by chromatography on silica gel (EtOAc/hexanes) afforded the title product. R^{I}, R^{II}, X, R′, and R″ are defined within the scope of the present disclosure. Synthesis of 1-(1,2-diphenylethyl)piperidine

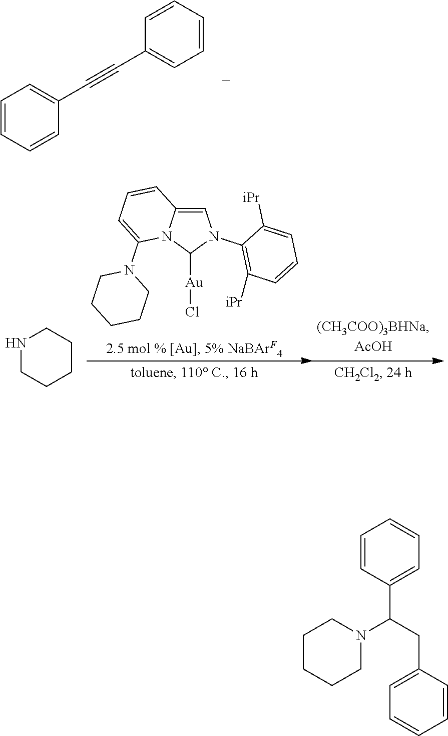

According to the general procedure, the reaction of diphenylacetylene (0.2 mmol, 1.0 equiv), piperidine (0.24 mmol, 1.2 equiv), 22 (0.005 mmol, 2.5 mol %), NaBAr^{F} (0.01 mmol, 5.0 mol %), toluene (0.5 M) at 110° C. and stirred for 16 hours, afforded after reduction with sodium triacetoxyborohydride (0.4 mmol, 2.0 equiv), acetic acid (0.4 mmol, 2.0 equiv) in CH₂Cl₂ (0.1 M), the title product in 99% yield (52.0 mg). ¹H NMR (500 MHz, CDCl₃) δ 7.24 (d, J=7.3 Hz, 2H), 7.20 (t, J=7.1 Hz, 1H), 7.13 (t, J=7.8 Hz, 4H), 7.08 (t, J=7.2 Hz, 1H), 7.02-6.97 (m, 2H), 3.69-3.54 (m, 1H), 3.40-3.26 (m, 1H), 3.02 (dd, J=13.3, 9.4 Hz, 1H), 2.45 (s, 4H), 1.58 (s, 4H), 1.37 (p, J=6.1 Hz, 2H). ¹³C NMR (126 MHz, CDCl₃) δ 139.91, 139.30, 129.39, 128.98, 127.88, 127.72, 126.92, 125.68, 72.38, 51.42, 39.12, 26.29, 24.61. Spectroscopic data matched literature values.

### General Procedure for Electrophilic Au(I) Hydroamination of Internal Alkynes with Hydrazine (General Procedure V

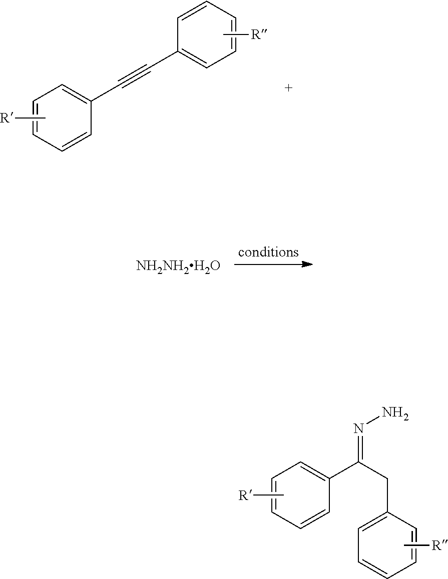

An oven-dried vial equipped with a stir bar was charged with alkyne (1.0 equiv), hydrazine monohydrate (1.2 equiv), [Au(NHC)Cl] catalyst (5 mol %), NaBAr^{F} (5 mol %), toluene (0.5 M) under argon at room temperature. The reaction mixture was placed in a preheated oil bath at 90° C. and stirred for 16 hours. After the indicated time, the reaction mixture was cooled down to room temperature and concentrated. The residue was analyzed by ¹H NMR (CDCl₃, 500 MHz) and GC-MS using internal standard. Purification by chromatography on silica gel (EtOAc/hexanes) afforded (1,2-diphenylethylidene)hydrazine. Catalysts: 22 (81% yield); 18 (78% yield); IPr—AuCl (80% yield). ¹H NMR (500 MHz, CDCl₃) δ 7.73 (d, J=7.3 Hz, 2H), 7.42-7.31 (m, 6H), 7.27 (s, 2H), 5.42 (s, 2H), 4.09 (s, 2H). ¹³C NMR (126 MHz, CDCl₃) δ 148.70, 139.14, 135.36, 129.13, 128.46, 128.19, 127.98, 126.90, 125.64, 32.45. Spectroscopic data matched literature values. R′ and R″ are defined within the scope of the present disclosure.

### General Procedure for Electrophilic Au(I) Hydroamination of Terminal alkynes with Aromatic Amines (General Procedure VI)

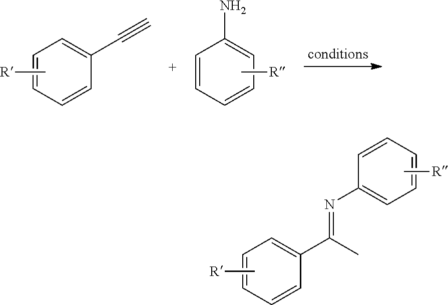

An oven-dried vial equipped with a stir bar was charged with alkyne (1.2 equiv), aniline (1.0 equiv), [Au(NHC)C₁] catalyst (0.1 mol %), NaBAr^{F} (0.2 mol %), toluene (1.5 M) under argon at room temperature. The reaction mixture was placed in a preheated oil bath at 80° C. and stirred for 16 hours. After the indicated time, the reaction mixture was cooled down to room temperature and concentrated. The residue was analyzed by ¹H NMR (CDCl₃, 500 MHz) and GC-MS using internal standard. Purification by chromatography on silica gel (EtOAc/hexanes) afforded N,1-diphenylethan-1-imine. Catalysts: 16 (89% yield); 22 (90% yield); 19 (83% yield); IPr—AuCl (83% yield). ¹H NMR (500 MHz, CDCl₃) δ 8.04-7.95 (m, 2H), 7.46 (d, J=7.1 Hz, 3H), 7.36 (t, J=7.8 Hz, 2H), 7.09 (t, J=7.4 Hz, 1H), 6.87-6.78 (m, 2H), 2.24 (s, 3H). ¹³C NMR (126 MHz, CDCl₃) δ 165.54, 151.68, 139.49, 130.50, 128.97, 128.39, 127.20, 123.25, 119.41, 17.41. Spectroscopic data matched literature values. R′ and R″ are defined within the scope of the present disclosure.

### General Procedure for Electrophilic Au(I) Hydration of Internal Alkynes (General Procedure VII)

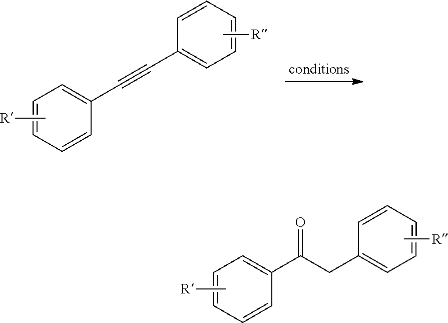

An oven-dried vial equipped with a stir bar was charged with alkyne (1.0 equiv), [Au(NHC)Cl] catalyst (0.1 mol %), AgSbF₆ (0.2 mol %), dioxane/H₂O=2/1 (1.0 M) at room temperature. The reaction mixture was placed in a preheated oil bath at 120° C. and stirred for 18 hours. After the indicated time, the reaction mixture was cooled down to room temperature and concentrated. The residue was analyzed by ¹H NMR (CDCl₃, 500 MHz) and GC-MS using internal standard. Purification by chromatography on silica gel (EtOAc/hexanes) afforded 1,2-diphenylethan-1-one. Catalysts: 19 (98% yield); IPr—AuCl (77% yield). ¹H NMR (500 MHz, CDCl₃) δ 8.14-8.00 (m, 2H), 7.63-7.55 (m, 1H), 7.55-7.44 (m, 2H), 7.42-7.23 (m, 5H), 4.31 (s, 2H). Spectroscopic data matched literature values. R′ and R″ are defined within the scope of the present disclosure.

### General Procedure for Pd-Catalyzed Amination of Aryl Chlorides (General Procedure VIII)

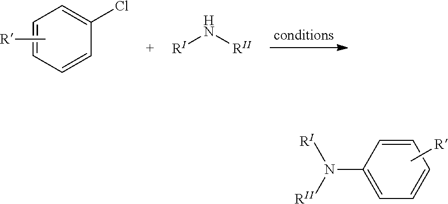

An oven-dried vial equipped with a stir bar was charged with aryl chloride (1.0 equiv), amine (2.0 equiv), [Pd—NHC] catalyst (3 mol %), dioxane (0.25 M) under argon at room temperature. LiHMDS (3.0 equiv, 1.0 M in THF) was added, the reaction mixture was placed in a preheated oil bath at 80° C. and stirred for 16 hours at 80° C. After the indicated time, the reaction mixture was cooled down to room temperature and extracted with EtOAc. The combined organic layers were dried, filtered and concentrated. The residue was analyzed by ¹H NMR (CDCl₃, 500 MHz) and GC-MS using internal standard. Purification by chromatography on silica gel (EtOAc/hexanes) afforded 4-(4-methoxyphenyl)morpholine. Catalysts: 26 (99% yield); 32 (99% yield); IPrCinPdCl (99% yield); [(cinnamyl)PdCl]₂ (0% yield). ¹H NMR (500 MHz, CDCl₃) δ 6.88 (q, J=8.9 Hz, 4H), 3.90-3.83 (m, 4H), 3.77 (s, 3H), 3.11-3.02 (m, 4H). ¹³C NMR (126 MHz, CDCl₃) δ 154.02, 145.65, 117.85, 114.53, 67.05, 55.59, 50.86. Spectroscopic data matched literature values.

### General Procedure for Pd-Catalyzed Kumada Cross-Coupling of Aryl Chlorides (General Procedure IX)

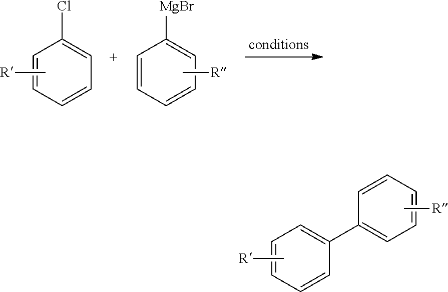

An oven-dried vial equipped with a stir bar was charged with aryl chloride (1.0 equiv), [Pd—NHC] catalyst (3 mol %), dioxane (0.25 M) under argon at room temperature. PhMgBr (2.0 equiv, 1.0 M in THF) was added, the reaction mixture was placed in a preheated oil bath at 80° C. and stirred for 16 hours at 80° C. After the indicated time, the reaction mixture was cooled down to room temperature and extracted by EtOAc. The combined organic layers were dried, filtered and concentrated. The residue was analyzed by ¹H NMR (CDCl₃, 500 MHz) and GC-MS using internal standard. Purification by chromatography on silica gel (EtOAc/hexanes) afforded 4-methoxy-1,1′-biphenyl. 26 (99% yield); 32 (99% yield); 33 (99% yield); IPrCinPdCl (99% yield); [(Cinnamyl)PdCl]₂ (0% yield). ¹H NMR (500 MHz, CDCl₃) δ 7.55 (dd, J=10.7, 8.1 Hz, 4H), 7.42 (t, J=7.7 Hz, 2H), 7.31 (t, J=7.4 Hz, 1H), 6.99 (d, J=8.7 Hz, 2H), 3.86 (s, 3H). ¹³C NMR (126 MHz, CDCl₃) δ 159.17, 140.86, 133.81, 128.73, 128.17, 126.76, 126.67, 114.23, 55.37. Spectroscopic data matched literature values. R′ and R″ are defined within the scope of the present disclosure.

### General Procedure for Pd-Catalyzed Arylation of Aroyl Chlorides (General Procedure X)

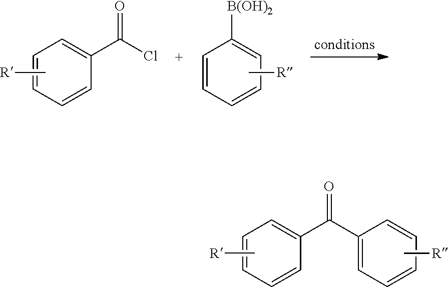

An oven-dried vial equipped with a stir bar was charged with aroyl chloride (1.0 equiv), boronic acid (2.0 equiv), K₂CO₃ (3.0 equiv), [Pd—NHC] catalyst (3 mol %), dioxane (0.25 M) under argon at room temperature. The reaction mixture was placed in a preheated oil bath at 130° C. and stirred for 16 hours at 130° C. After the indicated time, the reaction mixture was cooled down to room temperature, diluted with CH₂Cl₂, filtered and concentrated. The residue was analyzed by ¹H NMR (CDCl₃, 500 MHz) and GC-MS using internal standard. Purification by chromatography on silica gel (EtOAc/hexanes) afforded (4-methoxyphenyl)(p-tolyl)methanone. 26 (70% yield); IPrCinPdCl (72% yield); [(Cinnamyl)PdCl]₂ (25% yield). ¹H NMR (500 MHz, CDCl₃) δ 7.84 (d, J=8.8 Hz, 2H), 7.72 (d, J=8.2 Hz, 2H), 7.30 (d, J=8.0 Hz, 2H), 6.99 (d, J=8.8 Hz, 2H), 3.91 (s, 3H), 2.47 (s, 3H). Spectroscopic data matched literature values. R′ and R″ are defined within the scope of the present disclosure.

### General Procedure for Pd-Catalyzed Aminocarbonylation of Aryl Iodides (General Procedure XI)

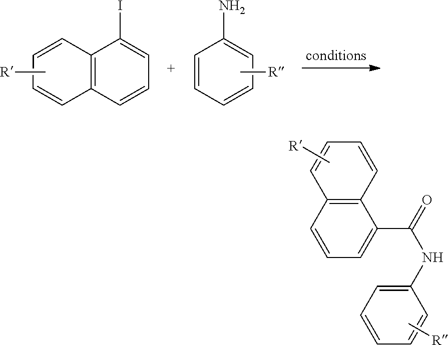

An oven-dried vial equipped with a stir bar was charged with aryl iodide (1.0 equiv), amine (1.2 equiv), K₃PO₄ (3.0 equiv), [Pd—NHC] catalyst (3 mol %), toluene (0.25 M) at room temperature. The vial was charged with CO gas (1 atm), placed in a preheated oil bath at 90° C. and stirred for 16 hours at 90° C. After the indicated time, the reaction mixture was cooled down to room temperature, diluted with CH₂Cl₂, filtered and concentrated. The residue was analyzed by ¹H NMR (CDCl₃, 500 MHz) and GC-MS using internal standard. Purification by chromatography on silica gel (EtOAc/hexanes) afforded N-Phenyl-1-naphthamide. 26 (99% yield); IPrCinPdCl (96% yield); [(Cinnamyl)PdCl]₂ (0% yield). ¹H NMR (500 MHz, CDCl₃) δ 8.37 (d, J=7.9 Hz, 1H), 7.97 (d, J=8.3 Hz, 1H), 7.91 (d, J=7.1 Hz, 1H), 7.72 (dd, J=20.8, 6.8 Hz, 4H), 7.60-7.54 (m, 2H), 7.52-7.48 (m, 1H), 7.40 (t, J=7.1 Hz, 2H), 7.19 (t, J=7.1 Hz, 1H). ¹³C NMR (126 MHz, CDCl₃) δ 167.55, 138.05, 133.79, 131.09, 129.20, 128.46, 127.41, 126.64, 125.28, 125.10, 124.78, 124.71, 119.97. Spectroscopic data matched literature values. R′ and R″ are defined within the scope of the present disclosure.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of formula (I) selected from the group consisting of:

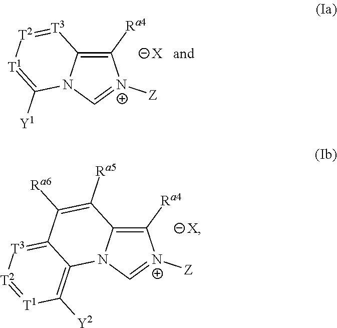

wherein:
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₅ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, R^{a3}, R^{a5}, and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
   wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen.

Embodiment 2 provides the compound of Embodiment 1, wherein at least one of the following occurs:
(a) T¹ is CR^{a1};
(b) T² is CR^{a2}; and
(c) T³ is CR^{a3}.

Embodiment 3 provides the compound of Embodiment 1 or 2, wherein Z is:

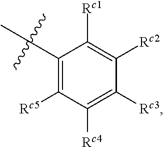

wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{c1}, R^{c2}, R^{c3}, R^{c4}, and R^{c5} may combine with the carbon atoms to which they are bound to form an optionally substituted C₅-C₁₂ cycloalkyl, optionally substituted C₂-C₁₂ heterocyclyl, or optionally substituted C₆-C₁₀ aryl.

Embodiment 4 provides the compound of any one of Embodiments 1-3, wherein at least one of the following occurs:
(a) at least one of R^{a1}, R^{a2}, and R^{a3} is H;
(b) at least two of R^{a1}, R^{a2}, and R^{a3} are H; and
(c) each of R^{a1}, R^{a2}, and R^{a3} are H.

Embodiment 5 provides the compound of any one of Embodiments 1-4, wherein X is selected from the group consisting of H, OS(═O)₂R^{A}, OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl

Embodiment 6 provides the compound of Embodiment 5, wherein X is Cl.

Embodiment 7 provides the compound of any one of Embodiments 1-6, wherein one of the following applies:
(a) R^{b1} and R^{b2} are each independently selected from the group consisting of H, methyl, ethyl, n-propyl, i-propyl, n-butyl, sec-butyl, and i-butyl; or
(b) R^{b1} and R^{b2} combine with the nitrogen atom to which they are bound to form a morpholinyl, piperidinyl, fluorenyl, or optionally substituted pyrazolyl.

Embodiment 8 provides the compound of any one of Embodiments 1-7, wherein Y¹ is selected from the group consisting of OMe, NMe₂, and NEt₂.

Embodiment 9 provides the compound of any one of Embodiments 1-7, wherein Y¹ is selected from the group consisting of

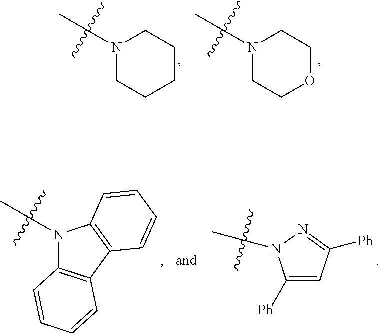 [SMILES: *N1CCCCC1 ; *N1CCOCC1 ; *n1c2ccccc2c2ccccc21 ; *n1nc(-c2ccccc2)cc1-c1ccccc1]

Embodiment 10 provides the compound of any one of Embodiments 1-7, wherein Y² is selected from the group consisting of NMe₂, NEt₂,

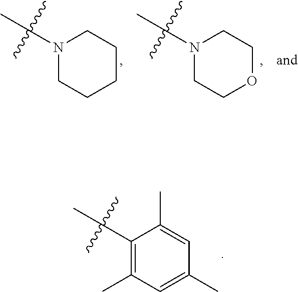

Embodiment 11 provides the compound of any one of Embodiments 3-10, wherein R^{c1} and R^{c5} are each independently selected from the group consisting of methyl, i-propyl, and diphenylmethyl.

Embodiment 12 provides the compound of Embodiment 11, wherein R^{c1} and R^{c5} are identical.

Embodiment 13 provides the compound of any one of Embodiments 3-12, wherein R^{c3} is selected from the group consisting of H and methyl.

Embodiment 14 provides the compound of any one of Embodiments 3-13, wherein R^{c2} and R^{c4} are each H.

Embodiment 15 provides the compound of any one of Embodiments 1-14, wherein Z is selected from the group consisting of

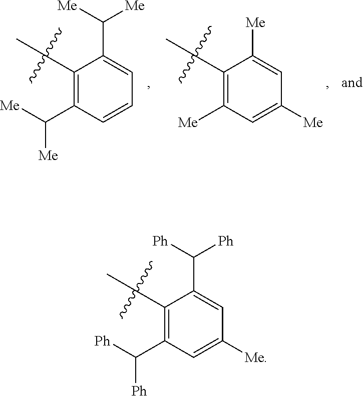 [SMILES: *c1c(C(C)C)cccc1C(C)C ; *c1c(C)cc(C)cc1C ; *c1c(C(c2ccccc2)c2ccccc2)cc(C)cc1C(c1ccccc1)c1ccccc1]

Embodiment 16 provides the compound of any one of Embodiments 1-15, which is selected from the group consisting of:
2-(2,6-diisopropylphenyl)-5-(dimethylamino)imidazo[1,5-a]pyridin-2-ium chloride;
5-(dimethylamino)-2-mesitylimidazo[1,5-a]pyridin-2-ium chloride;
2-(2,6-dibenzhydryl-4-methylphenyl)-5-(dimethylamino)imidazo[1,5-a]pyridin-2-ium chloride;
5-(diethylamino)-2-(2,6-diisopropylphenyl)imidazo[1,5-a]pyridin-2-ium chloride;
5-(diethylamino)-2-mesitylimidazo[1,5-a]pyridin-2-ium chloride;
2-(2,6-dibenzhydryl-4-methylphenyl)-5-(diethylamino)imidazo[1,5-a]pyridin-2-ium chloride;
2-(2,6-diisopropylphenyl)-5-(piperidin-1-yl)imidazo[1,5-a]pyridin-2-ium chloride;
2-mesityl-5-(piperidin-1-yl)imidazo[1,5-a]pyridin-2-ium chloride;
2-(2,6-dibenzhydryl-4-methylphenyl)-5-(piperidin-1-yl)imidazo[1,5-a]pyridin-2-ium chloride;
2-(2,6-diisopropylphenyl)-5-morpholinoimidazo[1,5-a]pyridin-2-ium chloride;
2-mesityl-5-morpholinoimidazo[1,5-a]pyridin-2-ium chloride;
2-(2,6-dibenzhydryl-4-methylphenyl)-5-morpholinoimidazo[1,5-a]pyridin-2-ium chloride;
2-(2,6-diisopropylphenyl)-5-(3,5-diphenyl-1H-pyrazol-1-yl)imidazo[1,5-a]pyridin-2-ium chloride;
5-(9H-carbazol-9-yl)-2-(2,6-diisopropylphenyl)imidazo[1,5-a]pyridin-2-ium chloride;
2-(2,6-diisopropylphenyl)-5-methoxyimidazo[1,5-a]pyridin-2-ium chloride;
2-(2,6-Diisopropylphenyl)-9-(dimethylamino)imidazo[1,5-a]quinolin-2-ium chloride;
2-(2,6-Diisopropylphenyl)-9-(diethylamino)imidazo[1,5-a]quinolin-2-ium chloride;
2-(2,6-Diisopropylphenyl)-9-(piperidin-1-yl)imidazo[1,5-a]quinolin-2-ium chloride;
2-(2,6-Diisopropylphenyl)-9-morpholinoimidazo[1,5-a]quinolin-2-ium chloride; 9-(Dimethylamino)-2-mesitylimidazo[1,5-a]quinolin-2-ium chloride; and
2-(2,6-Diisopropylphenyl)-9-(2,4,6-trimethylphenyl)imidazo[1,5-a]quinolin-2-ium chloride.

Embodiment 17 provides a method of preparing the compound of formula (Ia) of any one of Embodiments 1-9 and 11-16, the method comprising:
contacting a compound of formula (A)

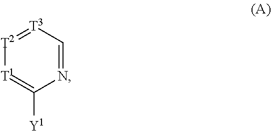

a compound of formula (B):

H₂N—Z (B), and
paraformaldehyde:

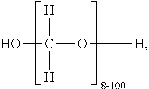

in the presence of an acid to form the compound of formula (Ia).

Embodiment 18 provides the method of Embodiment 17, wherein the contacting occurs in the presence of a solvent, wherein the solvent is optionally ethanol.

Embodiment 19 provides the method of Embodiment 17 or 18, wherein the contacting occurs at a temperature ranging from about 40° C. to about 80° C.

Embodiment 20 provides the method of any one of Embodiments 17-19, wherein the contacting for a period of time ranging from about 12 to about 36 h.

Embodiment 21 provides a compound of formula (II) selected from the group consisting of:

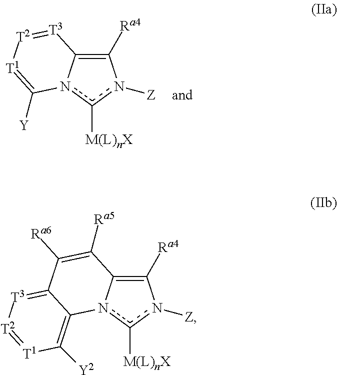

wherein:
M is a transition metal;
L is a ligand of M, wherein each occurrence of L can be the same or different;
each occurrence of [symbol] is a single or double bond,
   wherein no more than one [symbol] bonding a C atom and a N atom is a double bond;
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
   wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein each optional substituent in each of R^{a1}, R^{a2}, R^{a3}, R^{a5} and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
   wherein R^{b1} and R^{b2} may combine with the atom to which they are bound to form an optionally substituted C₂-C₁₂ heterocyclyl,
   wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen; and
n is an integer which is selected from the group consisting of 0, 1, 2, and 3.

Embodiment 22 provides the compound of Embodiment 21, wherein at least one of the following occurs:
(a) T¹ is CR^{a1};
(b) T² is CR^{a2}; and
(c) T³ is CR^{a3}.

Embodiment 23 provides the compound of Embodiment 21 or 22, wherein Z is:

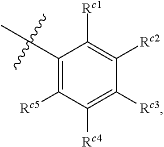

wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
   wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
   wherein two vicinal substituents selected from the group consisting of R^{c1}, R^{c2}, R^{c3}, R^{c4}, and R^{c5} may combine with the carbon atoms to which they are bound to form an optionally substituted C₅-C₁₂ cycloalkyl, optionally substituted C₂-C₁₂ heterocyclyl, or optionally substituted C₆-C₁₀ aryl.

Embodiment 24 provides the compound of any one of Embodiments 21-23, wherein M is selected from the group consisting of Cu, Ag, Au, Pd, Ni, Pt, Co, Rh, Ir, Fe, Ru, and Os.

Embodiment 25 provides the compound of Embodiment 24, wherein M is selected from the group consisting of Au, Pd, Rh, Ag and Cu.

Embodiment 26 provides the compound of any one of Embodiments 21-25, wherein X is selected from the group consisting of H, OS(═O)₂R^{A}, OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

Embodiment 27 provides the compound of Embodiment 26, wherein X is selected from the group consisting of Cl, trifluoromethanesulfonate (OT), bis(trifluoromethansulfonyl)amide (NTf₂), and allylbenzene anion (i.e., 3-phenylpropen-3-ide and/or 1-phenylpropen-3-ide).

Embodiment 28 provides the compound of any one of Embodiments 21-27, wherein L is selected from the group consisting of Y¹, Y², carbon monoxide (CO), optionally substituted C₂-C₁₂ alkene, and optionally substituted C₅-C₁₂ cycloalkene, wherein each optional substituent in the C₂-C₁₂ alkene and C₅-C₁₂ cycloalkene is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

Embodiment 29 provides the compound of any one of Embodiments 21-27, wherein L is selected from the group consisting of cyclooctadiene (COD) and carbon monoxide (CO).

Embodiment 30 provides the compound of any one of Embodiments 21-29, wherein at least one of the following occurs:
(a) at least one of R^{a1}, R^{a2}, and R^{a3} is H;
(b) at least two of R^{a1}, R^{a2}, and R^{a3} are H; and
(c) each of R^{a1}, R^{a2}, and R^{a3} are H.

Embodiment 31 provides the compound of any one of Embodiments 21-30, wherein one of the following applies:
(a) R^{b1} and R^{b2}, if present, are each independently selected from the group consisting of H, methyl, ethyl, n-propyl, i-propyl, n-butyl, sec-butyl, and i-butyl; or
(b) R^{b1} and R^{b2} combine with the nitrogen atom to which they are bound to form a morpholinyl or piperidinyl.

Embodiment 32 provides the compound of any one of Embodiments 21-31, wherein Y¹ is selected from the group consisting of OMe, NMe₂, and NEt₂.

Embodiment 33 provides the compound of any one of Embodiments 21-31, wherein Y¹ is selected from the group consisting of

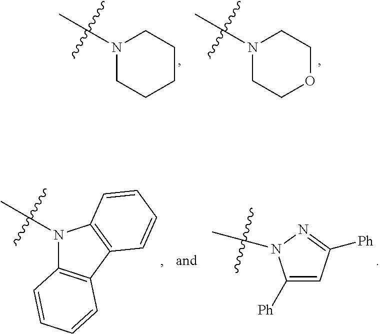 [SMILES: *N1CCCCC1 ; *N1CCOCC1 ; *n1c2ccccc2c2ccccc21 ; *n1nc(-c2ccccc2)cc1-c1ccccc1]

Embodiment 34 provides the compound of any one of Embodiments 21-31, wherein Y² is selected from the group consisting of NMe₂, NEt₂

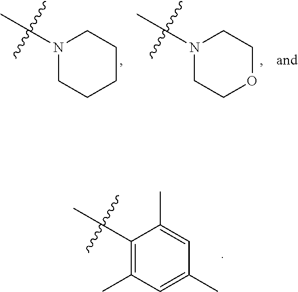

Embodiment 35 provides the compound of any one of Embodiments 23-34, wherein R^{c1} and R^{c5} are each independently selected from the group consisting of methyl, i-propyl, and diphenylmethyl.

Embodiment 36 provides the compound of any one of Embodiments 23-35, wherein R^{c1} and R^{c5} are identical.

Embodiment 37 provides the compound of any one of Embodiments 23-36, wherein R^{c3} is selected from the group consisting of H and methyl.

Embodiment 38 provides the compound of any one of Embodiments 23-37, wherein R^{c2} and R^{c4} are each H.

Embodiment 39 provides the compound of any one of Embodiments 21-38, wherein Z is selected from the group consisting of

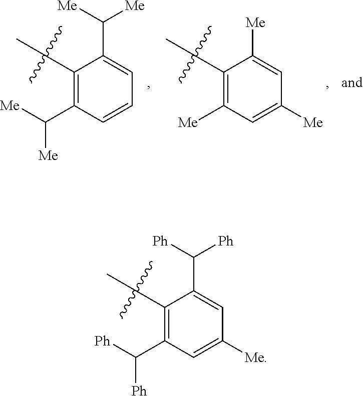 [SMILES: *c1c(C(C)C)cccc1C(C)C ; *c1c(C)cc(C)cc1C ; *c1c(C(c2ccccc2)c2ccccc2)cc(C)cc1C(c1ccccc1)c1ccccc1]

Embodiment 40 provides the compound of any one of Embodiments 21-39, which is selected from the group consisting of:
2-(2,6-diisopropylphenyl)-5-(dimethylamino)imidazo[1,5-a]pyridin-3-ylidene gold(I) chloride;
5-(dimethylamino)-2-mesitylimidazo[1,5-a]pyridin-3-ylidene gold(I) chloride;
2-(2,6-dibenzhydryl-4-methylphenyl)-5-(dimethylamino)imidazo[1,5-a]pyridin-3-ylidene gold(I) chloride;
5-(diethylamino)-2-(2,6-diisopropylphenyl)imidazo[1,5-a]pyridin-3-ylidene gold(I) chloride;
5-(diethylamino)-2-mesitylimidazo[1,5-a]pyridin-3-ylidene gold(I) chloride;
2-(2,6-dibenzhydryl-4-methylphenyl)-5-(diethylamino)imidazo[1,5-a]pyridin-3-ylidene gold(I) chloride;
2-(2,6-diisopropylphenyl)-5-(piperidin-1-yl)imidazo[1,5-a]pyridin-3-ylidene gold(I) chloride;
2-mesityl-5-(piperidin-1-yl)imidazo[1,5-a]pyridin-3-ylidene gold(I) chloride;
2-(2,6-dibenzhydryl-4-methylphenyl)-5-(piperidin-1-yl)imidazo[1,5-a]pyridin-3-ylidene gold(I) chloride;
2-(2,6-diisopropylphenyl)-5-methoxyimidazo[1,5-a]pyridin-3-ylidene gold(I) chloride;
cinnamyl[2-(2,6-diisopropylphenyl)-5-(dimethylamino)imidazo[1,5-a]pyridine-3-ylidene]chloropalladium(II);
cinnamyl[5-(dimethylamino)-2-mesitylimidazo[1,5-a]pyridin-3-ylidene]chloropalladium(II);
cinnamyl[2-(2,6-dibenzhydryl-4-methylphenyl)-5-(dimethylamino)imidazo[1,5-a]pyridin-3-ylidene]chloropalladium(II);
cinnamyl[5-(diethylamino)-2-(2,6-diisopropylphenyl)imidazo[1,5-a]pyridin-3-ylidene]chloropalladium(II);
cinnamyl[5-(diethylamino)-2-mesitylimidazo[1,5-a]pyridin-3-ylidene]chloropalladium(II);
cinnamyl[2-(2,6-dibenzhydryl-4-methylphenyl)-5-(diethylamino)imidazo[1,5-a]pyridin-3-ylidene]chloropalladium(II);
cinnamyl[2-(2,6-diisopropylphenyl)-5-(piperidin-1-yl)imidazo[1,5-a]pyridin-3-ylidene]chloropalladium(II);
cinnamyl[2-mesityl-5-(piperidin-1-yl)imidazo[1,5-a]pyridin-3-ylidene]chloropalladium(II);
cinnamyl[2-(2,6-dibenzhydryl-4-methylphenyl)-5-(piperidin-1-yl)imidazo[1,5-a]pyridin-3-ylidene]chloropalladium(II);
cinnamyl[2-(2,6-diisopropylphenyl)-5-morpholinoimidazo[1,5-a]pyridin-3-ylidene]chloropalladium(II);
2-(2,6-diisopropylphenyl)-5-(dimethylamino)imidazo[1,5-a]pyridin-3-ylidene silver(I) chloride;
2-(2,6-diisopropylphenyl)-5-(dimethylamino)imidazo[1,5-a]pyridin-3-ylidene copper(I) chloride;
2-(2,6-diisopropylphenyl)-5-(dimethylamino)imidazo[1,5-a]pyridin-3-ylidene(cyclooctadiene)rhodium(I) chloride;
2-(2,6-diisopropylphenyl)-5-(dimethylamino)imidazo[1,5-a]pyridin-3-ylidene(cyclooctadiene)rhodium(I) triflate;
2-(2,6-diisopropylphenyl)-5-(dimethylamino)imidazo[1,5-a]pyridin-3-ylidene(bis(carbonyl))rhodium(I) triflate;
2-(2,6-diisopropylphenyl)-5-(dimethylamino)imidazo[1,5-a]pyridin-3-ylidene gold(I) bis(trifluoromethanesulfonyl)amide;
2-(2,6-diisopropylphenyl)-5-(morpholin-4-yl)imidazo[1,5-a]pyridin-3-ylidene gold(I) chloride;
2-(2,4,6-trimethylphenyl)-5-(morpholin-4-yl)imidazo[1,5-a]pyridin-3-ylidene gold(I) chloride;
2-(2,6-Diisopropylphenyl)-9-(dimethylamino)imidazo[1,5-a]quinolin-3-ylidene silver(I) chloride;
2-(2,6-Diisopropylphenyl)-9-(diethylamino)imidazo[1,5-a]quinolin-3-ylidene silver(I) chloride;
2-(2,6-Diisopropylphenyl)-9-(piperidin-1-yl)imidazo[1,5-a]quinolin-3-ylidene silver(I) chloride;
2-(2,6-Diisopropylphenyl)-9-(morpholin-4-yl)imidazo[1,5-a]quinolin-3-ylidene silver(I) chloride;
2-(2,4,6-trimethylphenyl)-9-(dimethylamino)imidazo[1,5-a]quinolin-3-ylidene silver(I) chloride;
2-(2,6-Diisopropylphenyl)-9-(dimethylamino)imidazo[1,5-a]quinolin-3-ylidene gold(I) chloride;
2-(2,6-Diisopropylphenyl)-9-(diethylamino)imidazo[1,5-a]quinolin-3-ylidene gold(I) chloride;
2-(2,6-Diisopropylphenyl)-9-(piperidin-1-yl)imidazo[1,5-a]quinolin-3-ylidene gold(I) chloride;
2-(2,6-Diisopropylphenyl)-9-(morpholin-4-yl)imidazo[1,5-a]quinolin-3-ylidene gold(I) chloride;
2-(2,4,6-trimethylphenyl)-9-(dimethylamino)imidazo[1,5-a]quinolin-3-ylidene gold(I) chloride; and
2-(2,6-Diisopropylphenyl)-9-(2,4,6-trimethylphenyl)imidazo[1,5-a]quinolin-3-ylidene gold(I) chloride.

Embodiment 41 provides a method of promoting a reaction between a first reagent and an aryl iodide, the method comprising contacting the first reagent and the aryl iodide in the presence of the compound of any one of Embodiments 21-40 and optionally in the presence of a Lewis acid.

Embodiment 42 provides the method of Embodiment 41, wherein M is Au in the compound of any one of Embodiments 20-38.

Embodiment 43 provides the method of Embodiment 41 or 42, wherein the compound of any one of Embodiments 20-38 is present in an amount ranging from about 0.1 to about 10 mol.

Embodiment 44 provides the method of any one of Embodiments 41-43, wherein the first reagent is selected from the group consisting of optionally substituted C₆-C₁₀ aryl and optionally substituted C₂-C₁₀ heteroaryl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 45 provides the method of any one of Embodiments 41-44, wherein the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 46 provides the method of any one of Embodiments 41-45, wherein the Lewis acid is AgNTf₂.

Embodiment 47 provides the method of any one of Embodiments 41-46, wherein the contacting occurs in the presence of a solvent, wherein the solvent is optionally MeOH.

Embodiment 48 provides the method of any one of Embodiments 41-47, wherein the contacting occurs at a temperature ranging from about 40° C. to about 80° C.

Embodiment 49 provides the method of any one of Embodiments 41-48, wherein the contacting occurs for a period of time ranging from about 8 to about 24 h.

Embodiment 50 provides a method of promoting a reaction between an aniline and an aryl iodide, the method comprising contacting the aniline and the aryl iodide in the presence of the compound of any one of Embodiments 21-40 and optionally in the presence of a Lewis acid.

Embodiment 51 provides the method of Embodiment 50, wherein M is Au in the compound of any one of Embodiments 21-40.

Embodiment 52 provides the method of Embodiment 50 or 51, wherein the compound of any one of Embodiments 20-38 is present in an amount ranging from about 0.1 to about 10 mol.

Embodiment 53 provides the method of any one of Embodiments 50-52, wherein the aniline is selected from the group consisting of optionally substituted C₆-C₁₀ aryl and C₂-C₁₀ heteroaryl, wherein the aryl or heteroaryl is substituted with at least one NH₂, and wherein each optional substituent is at least one selected from the group consisting of of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 54 provides the method of any one of Embodiments 50-53, wherein the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 55 provides the method of any one of Embodiments 50-54, wherein the Lewis acid is AgNTf₂.

Embodiment 56 provides the method of any one of Embodiments 50-55, wherein the contacting occurs in the presence of a solvent, wherein the solvent is optionally MeOH.

Embodiment 57 provides the method of any one of Embodiments 50-56, wherein the contacting occurs at a temperature ranging from about 40° C. to about 80° C.

Embodiment 58 provides the method of any one of Embodiments 50-57, wherein the contacting occurs for a period of time ranging from about 8 to about 24 h.

Embodiment 59 provides a method of promoting a hydroamination reaction between an alkyne and an amine, the method comprising contacting the alkyne and the amine in the presence of the compound of any one of Embodiments 21-40 and optionally in the presence of a Lewis acid.

Embodiment 60 provides the method of Embodiment 59, wherein M is Au in the compound of any one of Embodiments 21-40.

Embodiment 61 provides the method of Embodiment 59 or 60, wherein the compound of any one of Embodiments 21-40 is present in an amount ranging from about 0.1 to about 10 mol.

Embodiment 62 provides the method of any one of Embodiments 59-61, wherein the alkyne is selected from the group consisting of optionally substituted C₂-C₁₂ alkynyl, optionally substituted C₈-C₁₂ aralkynyl, and optionally substituted C₄-C₁₂ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 63 provides the method of any one of Embodiments 59-62, wherein the amine is selected from the group consisting of optionally substituted C₄-C₁₂ heterocycloalkyl comprising at least one secondary amine, H₂N—NH₂, H₂N—N(optionally substituted C₁-C₆ alkyl)₂, H₂N—N(optionally substituted C₁-C₆ alkyl)(optionally substituted C₄-C₁₀ aryl), H₂N—N(optionally substituted C₄-C₁₀ aryl)₂, H₂N(optionally substituted C₁-C₆ alkyl), H₂N(optionally substituted C₄-C₁₀ aryl), HN(optionally substituted C₁-C₆ alkyl)(optionally substituted C₄-C₁₀ aryl), HN(optionally substituted C₄-C₁₀ aryl)₂, and NH(optionally substituted C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, halogen, CN, NO₂, C(═O)NH₂, C(═O)NH(C₁-C₆ alkyl), and C(═O)N(C₁-C₆ alkyl)₂.

Embodiment 64 provides the method of any one of Embodiments 59-63, wherein the Lewis acid is sodium tetrakis[3,5-bis(trifluoromethyl)phenyl]borate (NaBAr^{F}).

Embodiment 65 provides the method of any one of Embodiments 59-64, wherein the contacting occurs in the presence of a solvent.

Embodiment 66 provides the method of Embodiment 65, wherein the solvent is toluene.

Embodiment 67 provides the method of any one of Embodiments 59-66, wherein the contacting occurs at a temperature ranging from about 90° C. to about 110° C.

Embodiment 68 provides the method of any one of Embodiments 59-67, wherein the contacting occurs for a period of time ranging from about 8 to about 24 h.

Embodiment 69 provides the method of any one of Embodiments 59-68, wherein the contacting of the alkyne and amine provides an imine intermediate.

Embodiment 70 provides the method of Embodiment 69, wherein a reduction reaction is promoted by contacting the imine intermediate and a reducing agent.

Embodiment 71 provides the method of Embodiment 70, wherein the reducing agent is sodium triacetoxyborohydride (NaBH(OAc)₃).

Embodiment 72 provides a method of promoting hydration of an alkyne, the method comprising contacting the alkyne and water in the presence of the compound of any one of Embodiments 21-40 and optionally in the presence of a Lewis acid.

Embodiment 73 provides the method of Embodiment 72, wherein M is Au in the compound of any one of Embodiments 21-40.

Embodiment 74 provides the method of Embodiment 72 or 73, wherein the compound of any one of Embodiments 21-40 is present in an amount ranging from about 0.01 to about 1 mol.

Embodiment 75 provides the method of any one of Embodiments 72-74, wherein the alkyne is selected from the group consisting of optionally substituted C₂-C₁₂ alkynyl, optionally substituted C₈-C₁₂ aralkynyl, and optionally substituted C₄-C₁₂ heteroaralkynyl, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 76 provides the method of any one of Embodiments 72-75, wherein the Lewis acid is AgSbF₆.

Embodiment 77 provides the method of any one of Embodiments 72-76, wherein the contacting occurs in the presence of a solvent.

Embodiment 78 provides the method of Embodiment 77, wherein the solvent is a mixture of 1,4-dioxane and water.

Embodiment 79 provides the method of Embodiment 78, wherein the mixture of 1,4-dioxane and water has a ratio of about 10:1 to about 0.1:1 (1,4-dioxane:water).

Embodiment 80 provides the method of any one of Embodiments 72-79, wherein the contacting occurs at a temperature ranging from about 80° C. to about 100° C.

Embodiment 81 provides the method of any one of Embodiments 72-80, wherein the contacting occurs for a period of time ranging from about 8 to about 24 h.

Embodiment 82 provides a method of promoting a reaction between an aryl chloride and a lithium amide, the method comprising contacting the aryl chloride and the lithium amide in the presence of the compound of any one of Embodiments 21-40.

Embodiment 83 provides the method of Embodiment 82, wherein M is Pd in the compound of any one of Embodiments 21-40.

Embodiment 84 provides the method of Embodiment 82 or 83, wherein the compound of any one of Embodiments 21-40 is present in an amount ranging from about 0.1 to about 10 mol.

Embodiment 85 provides the method of any one of Embodiments 82-84, wherein the lithium amide is prepared by contacting an amine with a second lithium amide.

Embodiment 86 provides the method of Embodiment 85, wherein the second lithium amide is lithium hexamethyldisilazide (LiHMDS).

Embodiment 87 provides the method of Embodiment 85 or 86, wherein the amine is selected from the group consisting of optionally substituted C₄-C₁₂ heterocycloalkyl comprising at least one secondary amine, H₂N—NH₂, H₂N—N(optionally substituted C₁-C₆ alkyl)₂, H₂N—N(optionally substituted C₁-C₆ alkyl)(optionally substituted C₄-C₁₀ aryl), H₂N—N(optionally substituted C₄-C₁₀ aryl)₂, H₂N(optionally substituted C₁-C₆ alkyl), H₂N(optionally substituted C₄-C₁₀ aryl), HN(optionally substituted C₁-C₆ alkyl)(optionally substituted C₄-C₁₀ aryl), HN(optionally substituted C₄-C₁₀ aryl)₂, and NH(optionally substituted C₁-C₆ alkyl)₂, wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₄-C₁₀ heterocycloalkyl, C₂-C₆ alkenyl, phenyl, naphthyl, C₄-C₁₀ heteroaryl, N(C₁-C₆ alkyl)₂, halogen, CN, NO₂, C(═O)NH₂, C(═O)NH(C₁-C₆ alkyl), and C(═O)N(C₁-C₆ alkyl)₂.

Embodiment 88 provides the method of any one of Embodiments 82-87, wherein the aryl chloride is selected from the group consisting of optionally substituted C₆-C₁₀ aryl and optionally substituted C₂-C₁₀ heteroaryl, wherein the aryl or heteroaryl is substituted with at least one chlorine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 89 provides the method of any one of Embodiments 82-88, wherein the contacting occurs in the presence of a solvent.

Embodiment 90 provides the method of Embodiment 89, wherein the solvent is 1,4-dioxane.

Embodiment 91 provides the method of any one of Embodiments 82-90, wherein the contacting occurs at a temperature ranging from about 80° C. to about 100° C.

Embodiment 92 provides the method of any one of Embodiments 82-91, wherein the contacting occurs for a period of time ranging from about 12 to about 24 h.

Embodiment 93 provides a method of promoting a reaction between an aryl chloride and an arylmagnesium halide, the method comprising contacting the aryl chloride and the arylmagnesium halide in the presence of the compound of any one of Embodiments 21-40.

Embodiment 94 provides the method of Embodiment 93, wherein M is Pd in the compound of any one of Embodiments 21-40.

Embodiment 95 provides the method of Embodiment 93 or 94, wherein the compound of any one of Embodiments 21-40 has a concentration of about 0.1 to about 10 mol %.

Embodiment 96 provides the method of any one of Embodiments 93-95, wherein the aryl chloride is selected from the group consisting of optionally substituted C₆-C₁₀ aryl and optionally substituted C₂-C₁₀ heteroaryl, wherein the aryl or heteroaryl is substituted with at least one chlorine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, and NO₂.

Embodiment 97 provides the method of any one of Embodiments 93-96, wherein the arylmagnesium halide is selected from the group consisting of optionally substituted C₆-C₁₀ aryl and optionally substituted C₂-C₁₀ heteroaryl, wherein the aryl or heteroaryl is substituted with a magnesium halide moiety, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, and NO₂.

Embodiment 98 provides the method of any one of Embodiments 93-97, wherein the contacting occurs in the presence of a solvent.

Embodiment 99 provides the method of Embodiment 98, wherein the solvent is 1,4-dioxane.

Embodiment 100 provides the method of any one of Embodiments 93-99, wherein the contacting occurs at a temperature ranging from about 80° C. to about 100° C.

Embodiment 101 provides the method of any one of Embodiments 93-100, wherein the contacting occurs for a period of time ranging from about 8 to about 24 h.

Embodiment 102 provides a method of promoting a reaction between an aroyl chloride and an aryl boronic acid, the method comprising contacting the aroyl chloride and the aryl boronic acid in the presence of the compound of any one of Embodiments 21-40 and a base.

Embodiment 103 provides the method of Embodiment 102, wherein M is Pd in the compound of any one of Embodiments 21-40.

Embodiment 104 provides the method of Embodiment 102 or 103, wherein the compound of any one of Embodiments 21-40 is present in an amount ranging from about 0.1 to about 10 mol %.

Embodiment 105 provides the method of any one of Embodiments 102-104, wherein the aroyl chloride is selected from the group consisting of optionally substituted C₆-C₁₀ aryl and optionally substituted C₂-C₁₀ heteroaryl, wherein the aryl or heteroaryl is substituted with a C(═O)C₁ moiety, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, N(C₁-C₆ alkyl)₂, halogen, CN, NO₂, C(═O)O(C₁-C₆ alkyl), and C(═O)N(C₁-C₆ alkyl)₂.

Embodiment 106 provides the method of any one of Embodiments 102-105, wherein the aryl boronic acid is selected from the group consisting of optionally substituted C₆-C₁₀ aryl boronic acid and optionally substituted C₄-C₁₀ heteroaryl boronic acid, wherein each optional substituent is at least one selected the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 107 provides the method of any one of Embodiments 102-106, wherein the base is K₂CO₃.

Embodiment 108 provides the method of any one of Embodiments 102-107, wherein the reaction occurs in the presence of a solvent.

Embodiment 109 provides the method of Embodiment 108, wherein the solvent is 1,4-dioxane.

Embodiment 110 provides the method of any one of Embodiments 102-109, wherein the contacting occurs at a temperature ranging from about 80° C. to about 100° C.

Embodiment 111 provides the method of any one of Embodiments 102-110, wherein the contacting occurs for a period of time ranging from about 8 h to about 24 h.

Embodiment 112 provides a method of promoting a reaction between an aryl iodide, an aniline, and carbon monoxide (CO), the method comprising contacting the aryl iodide, the aniline, and the CO in the presence of the compound of any one of Embodiments 21-40 and a base.

Embodiment 113 provides the method of Embodiment 112, wherein M is Pd in the compound of any one of Embodiments 21-40.

Embodiment 114 provides the method of Embodiment 112 or 113, wherein the compound of any one of Embodiments 21-40 is present in an amount ranging from about 0.1 to about 10.0 mol %.

Embodiment 115 provides the method of any one of Embodiments 112-114, wherein the aryl iodide is selected from the group consisting of optionally substituted C₆-C₁₀ aryl and optionally substituted C₂-C₁₀ heteroaryl, wherein the aryl or heteroaryl is substituted with at least one iodine atom, and wherein each optional substituent is at least one selected from the group consisting of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 116 provides the method of any one of Embodiments 112-115, wherein the aniline is selected from the group consisting of optionally substituted C₆-C₁₀ aryl and C₂-C₁₀ heteroaryl, wherein the aryl or heteroaryl is substituted with at least one NH₂, and wherein each optional substituent is at least one selected from the group consisting of of C₁-C₆ alkoxy, C₁-C₆ haloalkoxy, C₁-C₆, haloalkyl, C₁-C₆ alkyl, C₃-C₁₂ cycloalkyl, C₂-C₁₀ heterocyclyl, C₂-C₆ alkenyl, phenyl, naphthyl, NH₂, N(C₁-C₆ alkyl)₂, halogen, OH, CN, NO₂, C(═O)OH, C(═O)O(C₁-C₆ alkyl), C(═O)NH₂, C(═O)NH(C₁-C₆ alkyl), and C(═O)N(C₁-C₆ alkyl)₂.

Embodiment 117 provides the method of any one of Embodiments 112-116, wherein the CO has a pressure ranging from about 0.1 to about 10 atm.

Embodiment 118 provides the method of any one of Embodiments 112-117, wherein the base is K₃PO₄.

Embodiment 119 provides the method of any one of Embodiments 112-118, wherein the contacting occurs in the presence of a solvent.

Embodiment 120 provides the method of Embodiment 119, wherein the solvent is toluene.

Embodiment 121 provides the method of any one of Embodiments 112-120, wherein the contacting occurs at a temperature ranging from about 90° C. to about 110° C.

Embodiment 122 provides the method of any one of Embodiments 112-121, wherein the contacting occurs for a period of time ranging from about 8 to about 24 h.

Embodiment 123 provides a method of preparing 2-(2,6-diisopropylphenyl)-5-(dimethylamino)imidazo[1,5-a]pyridin-2-ium chloride) (1):

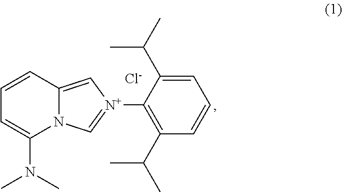 [SMILES: CC(C)c1cccc(C(C)C)c1-[n+]1cc2cccc(N(C)C)n2c1 ; [Cl-]]

the method comprising reacting (E)-6-(((2,6-diisopropylphenyl)imino)methyl)-N,N-dimethylpyridin-2-amine (A):

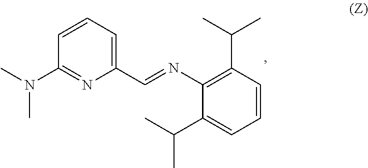 [SMILES: CC(C)c1cccc(C(C)C)c1/N=C/c1cccc(N(C)C)n1]

and paraformaldehyde so as to generate a first reaction system comprising (1).

Embodiment 124 provides the method of Embodiment 123, wherein the reaction of (Z) and paraformaldehyde is performed in the presence of a solvent.

Embodiment 125 provides the method of Embodiment 124, wherein the solvent is EtOH.

Embodiment 126 provides the method of any one of Embodiments 123-125, further comprising hydrochloric acid (HCl).

Embodiment 127 provides the method of any one of Embodiments 123-126, wherein the reaction of (Z) and paraformaldehyde is performed at a temperature of about 70° C.

Embodiment 128 provides the method of any one of Embodiments 123-127, wherein the (Z) is prepared by reacting 6-(dimethylamino)picolinaldehyde (Y):

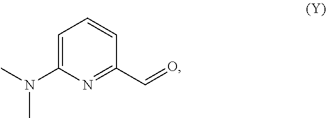

and 2,6-diisopropylaniline (X):

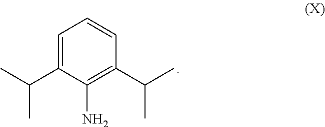

Embodiment 129 provides the method of Embodiment 128, wherein the reaction of (Y) and (X) is performed in the presence of a solvent.

Embodiment 130 provides the method of Embodiment 129, wherein the solvent is EtOH.

Embodiment 131 provides the method of any one of Embodiments 128-130, wherein the reaction of (Y) and (X) is performed at a temperature of about 90° C.

Embodiment 132 provides the method of any one of Embodiments 128-131, wherein the (Y) is prepared by reacting 6-bromo-N,N-dimethylpyridin-2-amine (W):

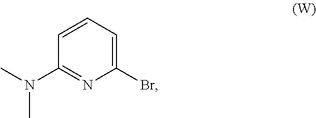

and an organolithium reagent to form a lithiated intermediate,
and contacting the lithiated intermediate with a formylating reagent.

Embodiment 133 provides the method of Embodiment 132, wherein the reaction of (W) and the organolithium reagent is performed in the presence of a solvent.

Embodiment 134 provides the method of Embodiment 133, wherein the solvent is tetrahydrofuran (THF).

Embodiment 135 provides the method of any one of Embodiments 132-134, wherein the organolithium reagent is n-butyllithium (n-BuLi).

Embodiment 136 provides the method of any one of Embodiments 132-135, wherein the reaction of (W) and the organolithium reagent is performed at about −78° C.

Embodiment 137 provides the method of any one of Embodiments 132-136, wherein the reaction of the lithiated intermediate and the formylating reagent is performed in the presence of a solvent.

Embodiment 138 provides the method of Embodiment 137, wherein the solvent is tetrahydrofuran (THF).

Embodiment 139 provides the method of any one of Embodiments 132-138, wherein the formylating agent is dimethylformamide (DMF).

Embodiment 140 provides the method of any one of Embodiments 132-139, wherein the contacting occurs at a temperature of about −78° C.

Embodiment 141 provides the method of any one of Embodiments 132-136, wherein the (W) is prepared by reacting (V):

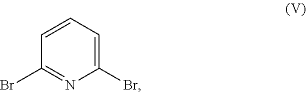

and dimethylamine (HNMe₂),
in the presence of a base.

Embodiment 142 provides the method of Embodiment 141, wherein the reaction of (V) and dimethylamine is performed in the presence of a solvent.

Embodiment 143 provides the method of Embodiment 142, wherein the solvent is acetonitrile (ACN).

Embodiment 144 provides the method of any one of Embodiments 141-143, wherein the base is K₂CO₃.

Embodiment 145 provides the method of any one of Embodiments 141-144, wherein the reaction of (V) and dimethylamine is performed at a temperature of about 100° C.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

What is claimed is:

1. A compound of formula (I) selected from the group consisting of: 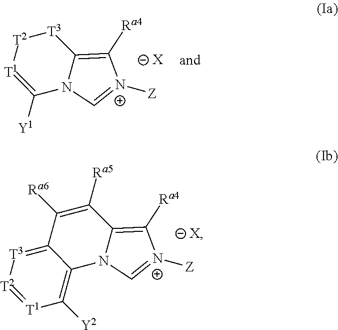
wherein:
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein each optional substituent in each of R^{a1}, R^{a2}, R^{a3}, R^{a5}, and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{a1}, R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein R^{b1} and R^{b2} may combine with the atom to which they are bound to form an optionally substituted C₂-C₁₂ heterocyclyl,
wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl; and
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen.

2. The compound of claim 1, wherein at least one of the following occurs:
(a) T¹ is CR^{a1};
(b) T² is CR^{a2};
(c) T³ is CR^{a3};
(d) at least one of R^{a1}, R^{a2}, and R^{a3} is H:
(e) at least two of R^{a1}, R^{a2}, and R^{a3} are H:
(f) each of R^{a1}, R^{a2}, and R^{a3} are H;
(g) X is Cl; and
(h) R^{b1} and R^{b2} are each independently selected from the group consisting of H, methyl, ethyl, n-propyl, i-propyl, n-butyl, sec-butyl, and i-butyl, or R^{b1} and R^{b2} combine with the nitrogen atom to which they are bound to form a morpholinyl, piperidinyl, fluorenyl, or optionally substituted pyrazolyl.

3. The compound of claim 1, wherein Z is: 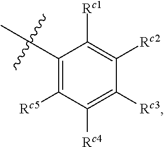
wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{c1}, R^{c2}, R^{c3}, R^{c4}, and R^{c5} may combine with the carbon atoms to which they are bound to form an optionally substituted C₅-C₁₂ cycloalkyl, optionally substituted C₂-C₁₂ heterocyclyl, or optionally substituted C₆-C₁₀ aryl.

4-7. (canceled)

8. The compound of claim 1, wherein one of the following applies:
(a) Y¹ is selected from the group consisting of OMe, NMe₂, NEt₂, 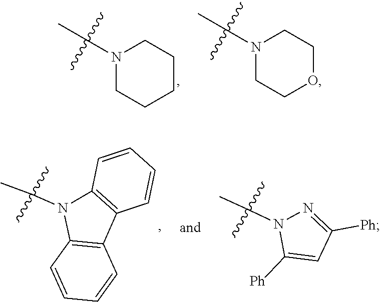
or
(b) Y² is selected from the group consisting of NMe₂, NEt₂, and

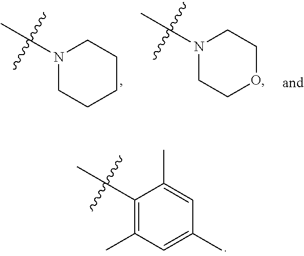

9-10. (canceled)

11. The compound of claim 3, wherein at least one of the following applies:
(a) R^{c1} and R^{c5} are each independently selected from the group consisting of methyl, i-propyl, and diphenylmethyl;
(b) R^{c1} and R^{c5} are identical;
(c) R^{c3} is selected from the group consisting of H and methyl; and
(d) R^{c2} and R^{c3} are each H.

12-14. (canceled)

15. The compound of claim 1, wherein Z is selected from the group consisting of

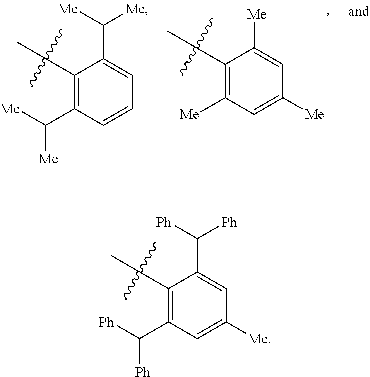 [SMILES: *c1c(C(C)C)cccc1C(C)C ; *c1c(C)cc(C)cc1C ; *c1c(C(c2ccccc2)c2ccccc2)cc(C)cc1C(c1ccccc1)c1ccccc1]

16. The compound of claim 1, which is selected from the group consisting of:
2-(2,6-diisopropylphenyl)-5-(dimethylamino)imidazo[1,5-a]pyridin-2-ium chloride;
5-(dimethylamino)-2-mesitylimidazo[1,5-a]pyridin-2-ium chloride;
2-(2,6-dibenzhydryl-4-methylphenyl)-5-(dimethylamino)imidazo[1,5-a]pyridin-2-ium chloride;
5-(diethylamino)-2-(2,6-diisopropylphenyl)imidazo[1,5-a]pyridin-2-ium chloride;
5-(diethylamino)-2-mesitylimidazo[1,5-a]pyridin-2-ium chloride;
2-(2,6-dibenzhydryl-4-methylphenyl)-5-(diethylamino)imidazo[1,5-a]pyridin-2-ium chloride;
2-(2,6-diisopropylphenyl)-5-(piperidin-1-yl)imidazo[1,5-a]pyridin-2-ium chloride;
2-mesityl-5-(piperidin-1-yl)imidazo[1,5-a]pyridin-2-ium chloride;
2-(2,6-dibenzhydryl-4-methylphenyl)-5-(piperidin-1-yl)imidazo[1,5-a]pyridin-2-ium chloride;
2-(2,6-diisopropylphenyl)-5-morpholinoimidazo[1,5-a]pyridin-2-ium chloride;
2-mesityl-5-morpholinoimidazo[1,5-a]pyridin-2-ium chloride;
2-(2,6-dibenzhydryl-4-methylphenyl)-5-morpholinoimidazo[1,5-a]pyridin-2-ium chloride;
2-(2,6-diisopropylphenyl)-5-(3,5-diphenyl-1H-pyrazol-1-yl)imidazo[1,5-a]pyridin-2-ium chloride;
5-(9H-carbazol-9-yl)-2-(2,6-diisopropylphenyl)imidazo[1,5-a]pyridin-2-ium chloride;
2-(2,6-diisopropylphenyl)-5-methoxyimidazo[1,5-a]pyridin-2-ium chloride;
2-(2,6-Diisopropylphenyl)-9-(dimethylamino)imidazo[1,5-a]quinolin-2-ium chloride;
2-(2,6-Diisopropylphenyl)-9-(diethylamino)imidazo[1,5-a]quinolin-2-ium chloride;
2-(2,6-Diisopropylphenyl)-9-(piperidin-1-yl)imidazo[1,5-a]quinolin-2-ium chloride;
2-(2,6-Diisopropylphenyl)-9-morpholinoimidazo[1,5-a]quinolin-2-ium chloride;
9-(Dimethylamino)-2-mesitylimidazo[1,5-a]quinolin-2-ium chloride; and
2-(2,6-Diisopropylphenyl)-9-(2,4,6-trimethylphenyl)imidazo[1,5-a]quinolin-2-ium chloride.

17. A method of preparing the compound of formula (Ia) of claim 1, the method comprising:
contacting a compound of formula (A): 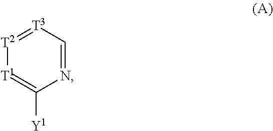
a compound of formula (B):
H₂N—Z (B), and
paraformaldehyde: 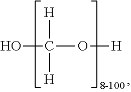
in the presence of an acid to form the compound of formula (Ia).

18. The method of claim 17, wherein at least one of the following applies:
(a) the contacting occurs in the presence of a solvent, wherein the solvent is optionally ethanol;
(b) the contacting occurs at a temperature ranging from about 40° C. to about 80° C.; and
(c) the contacting for a period of time ranging from about 12 to about 36 h.

19-20. (canceled)

21. A compound of formula (II) selected from the group consisting of: 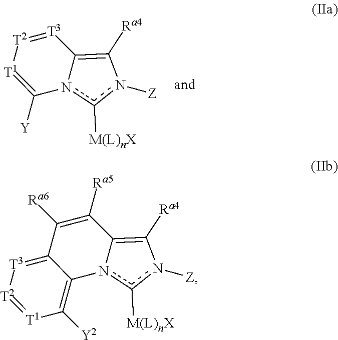
wherein:
M is a transition metal;
L is a ligand of M, wherein each occurrence of L can be the same or different;
each occurrence of [symbol] is a single or double bond,
wherein no more than [symbol] bonding a C atom and a N atom is a double bond;
T¹ is N or CR^{a1};
T² is N or CR^{a2};
T³ is N or CR^{a3};
X is a counter anion;
Y¹, if present, is selected from the group consisting of OR^{b1} and N(R^{b1})(R^{b2});
Y², if present, is selected from the group consisting of N(R^{b1})(R^{b2}) and optionally substituted phenyl;
Z is selected from the group consisting of optionally substituted C₆-C₁₀ aryl and optionally substituted C₂-C₈ heteroaryl,
wherein each optional substituent in Z is independently at least one selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein the C₁-C₆ alkyl, C₃-C₈ cycloalkyl, C₂-C₁₂ heterocyclyl, C₂-C₆ alkenyl, benzyl, phenyl, and naphthyl are each optionally substituted with at least one substituent selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{a1}, R^{a2}, R^{a3}, R^{a5}, and R^{a6}, if present, are each independently selected from the group consisting of H, halogen, C₁-C₃ haloalkyl, C₁-C₃ alkoxy, C₁-C₃ haloalkoxy, CN, NO₂, OR^{A}N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein each optional substituent in each of R^{a1}, R^{a2}, R^{a3}, R^{a5}, and R^{a6} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of Rai R^{a2}, R^{a3}, R^{a5}, and R^{a6} may combine with the carbon atoms to which they are bound to form an optionally substituted C₅-C₁₂ cycloalkyl, optionally substituted C₂-C₁₂ heterocyclyl, or optionally substituted C₆-C₁₀ aryl;
R^{a4} is selected from the group consisting of H, C₁-C₆ alkyl, C₆-C₁₀ aryl, and C₂-C₁₂ heterocyclyl;
R^{b1} and R^{b2} are each independently selected from the group consisting of H, optionally substituted C₁-C₆ alkyl, optionally substituted C₃-C₈ cycloalkyl, optionally substituted C₂-C₈ heterocyclyl, optionally substituted C₂-C₆ alkenyl, optionally substituted benzyl, optionally substituted phenyl, and optionally substituted naphthyl,
wherein R^{b1} and R^{b2} may combine with the atom to which they are bound to form an optionally substituted C₂-C₁₂ heterocyclyl,
wherein each optional substituent in each of R^{b1} and R^{b2} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl;
R^{A} and R^{B} are each independently selected from the group consisting of H, C₁-C₆ alkyl, C₁-C₃ haloalkyl, C₂-C₆ alkenyl, benzyl, naphthyl, C₄-C₁₀ heteroaryl, and phenyl, wherein each substituent in R^{A} and R^{B} is optionally substituted with at least one substituent selected from the group consisting of CN, NO₂, C₁-C₃ haloalkyl, C₁-C₃ alkoxy, C₁-C₃ haloalkoxy, C₁-C₃ alkyl, and halogen; and
n is an integer which is selected from the group consisting of 0, 1, 2, and 3.

22. The compound of claim 21, wherein at least one of the following occurs:
(a) T¹ is CR^{a1};
(b) T² is CR^{a2};
(c) T³ is CR^{a3};
(d) at least one of R^{a1}, R^{a2}, and R^{a3} is H;
(e) at least two of R^{a1}, R^{a2}, and R^{a3} are H;
(f) each of R^{a1}, R^{a2}, and R^{a3} are H; and
(g) R^{b1} and R^{b2} are each independently selected from the group consisting of H, methyl, ethyl, n-propyl, i-propyl, n-butyl, sec-butyl, and i-butyl, or R^{b1} and R^{b2} combine with the nitrogen atom to which they are bound to form a morpholinyl, piperidinyl, fluorenyl, or optionally substituted pyrazolyl.

23. The compound of claim 21, wherein Z is: 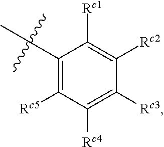
wherein:
R^{c1}, R^{c2}, R^{c3}, R^{c4}, and R^{c5} are each independently selected from the group consisting of H, halogen, C₁-C₃ haloalkyl, C₁-C₃ alkoxy, C₁-C₃ haloalkoxy, CN, NO₂, OR^{A}, N(R^{A})(R^{B}), C(═O)R^{A}, C(═O)N(R^{A})(R^{B}), C(═O)OR^{B}, N(R^{A})S(═O)₂R^{B}, S(═O)₂N(R^{A}), optionally substituted C₁-C₆ alkyl, optionally substituted C₃-C₈ cycloalkyl, optionally substituted C₂-C₁₂ heterocyclyl, optionally substituted C₂-C₆ alkenyl, optionally substituted benzyl, optionally substituted phenyl, and optionally substituted naphthyl,
wherein each optional substituent in each of R^{c1}, R^{c2}, R^{c3}, R^{c4}, and R^{c5} is independently selected from the group consisting of halogen, C₁-C₃ haloalkyl, C₁-C₃ alkoxy, C₁-C₃ haloalkoxy, C₁-C₃ alkyl, C₂-C₆ alkenyl, benzyl, phenyl, and naphthyl, and C₂-C₁₂ heterocyclyl,
wherein two vicinal substituents selected from the group consisting of R^{c1}, R^{c2}, R^{c3}, R^{c4}, and R^{c5} may combine with the carbon atoms to which they are bound to form an optionally substituted C₅-C₁₂ cycloalkyl, optionally substituted C₂-C₁₂ heterocyclyl, or optionally substituted C₆-C₁₀ aryl.

24. The compound of claim 21, wherein at least one of the following applies:
(a) M is selected from the group consisting of Cu, Ag, Au, Pd, Ni, Pt, Co, Rh, Ir, Fe, Ru, and Os,
(b) X is selected from the group consisting of H, OS(═O)₂R^{A}, OC(═O)R^{A}, N(C(═O)R^{A})₂, halogen, tetracoordinate boronate, hexacoordinate phosphorus, optionally substituted C₆-C₁₀ aryl, and optionally substituted C₂-C₁₀ heteroaryl, wherein each optional substituent in the C₆-C₁₀ aryl and C₂-C₈ heteroaryl is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl; and
(c) L is selected from the group consisting of Y¹, Y², carbon monoxide (CO), optionally substituted C₂-C₁₂ alkene, and optionally substituted C₅-C₁₂ cycloalkene, wherein each optional substituent in the C₂-C₁₂ alkene and C₅-C₁₂ cycloalkene is independently selected from the group consisting of a halogen, CN, NO₂, C₁-C₃ haloalkyl, C₁-C₃ alkoxy, C₁-C₃ haloalkoxy, C₁-C₃ alkyl, C₃-C₈ cycloalkyl, phenyl, and C₂-C₈ heterocyclyl.

25-26. (canceled)

27. The compound of claim 24, wherein at least one of the following applies:
(a) X is selected from the group consisting of Cl, trifluoromethanesulfonate (OTf), bis(trifluoromethansulfonyl)amide (NTf₂), and allylbenzene anion (i.e., 3-phenylpropen-3-ide and/or 1-phenylpropen-3-ide),
(b) L is selected from the group consisting of cyclooctadiene (COD) and carbon monoxide (CO).

28-31. (canceled)

32. The compound of claim 21, wherein one of the following applies:
(a) Y¹ is selected from the group consisting of OMe, NMe₂, NEt₂, 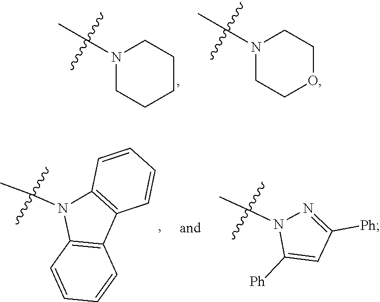
or
(b) Y² is selected from the group consisting of NMe₂, NEt₂,

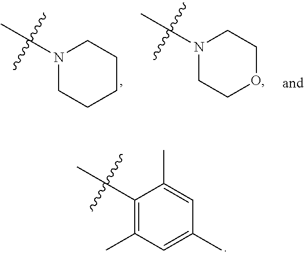

33-34. (canceled)

35. The compound of claim 23, wherein at least one of the following applies:
(a) R^{c1} and R^{c5} are each independently selected from the group consisting of methyl, i-propyl, and diphenylmethyl;
(b) R^{c1} and R^{c5} are identical;
(c) R^{c3} is selected from the group consisting of H and methyl; and
(d) R^{c2} and R^{c3} are each H.

36-38. (canceled)

39. The compound of claim 21, wherein Z is selected from the group consisting of

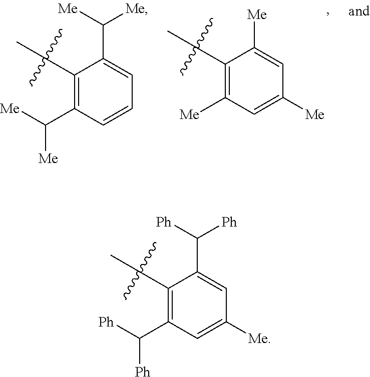 [SMILES: *c1c(C(C)C)cccc1C(C)C ; *c1c(C)cc(C)cc1C ; *c1c(C(c2ccccc2)c2ccccc2)cc(C)cc1C(c1ccccc1)c1ccccc1]

40. The compound of any one of claim 21, which is selected from the group consisting of:
2-(2,6-diisopropylphenyl)-5-(dimethylamino)imidazo[1,5-a]pyridin-3-ylidene gold(I) chloride;
5-(dimethylamino)-2-mesitylimidazo[1,5-a]pyridin-3-ylidene gold(I) chloride;
2-(2,6-dibenzhydryl-4-methylphenyl)-5-(dimethylamino)imidazo[1,5-a]pyridin-3-ylidene gold(I) chloride;
5-(diethylamino)-2-(2,6-diisopropylphenyl)imidazo[1,5-a]pyridin-3-ylidene gold(I) chloride;
5-(diethylamino)-2-mesitylimidazo[1,5-a]pyridin-3-ylidene gold(I) chloride;
2-(2,6-dibenzhydryl-4-methylphenyl)-5-(diethylamino)imidazo[1,5-a]pyridin-3-ylidene gold(I) chloride;
2-(2,6-diisopropylphenyl)-5-(piperidin-1-yl)imidazo[1,5-a]pyridin-3-ylidene gold(I) chloride;
2-mesityl-5-(piperidin-1-yl)imidazo[1,5-a]pyridin-3-ylidene gold(I) chloride;
2-(2,6-dibenzhydryl-4-methylphenyl)-5-(piperidin-1-yl)imidazo[1,5-a]pyridin-3-ylidene gold(I) chloride;
2-(2,6-diisopropylphenyl)-5-methoxyimidazo[1,5-a]pyridin-3-ylidene gold(I) chloride;
cinnamyl[2-(2,6-diisopropylphenyl)-5-(dimethylamino)imidazo[1,5-a]pyridine-3-ylidene]chloropalladium(II);
cinnamyl[5-(dimethylamino)-2-mesitylimidazo[1,5-a]pyridin-3-ylidene]chloropalladium(II);
cinnamyl[2-(2,6-dibenzhydryl-4-methylphenyl)-5-(dimethylamino)imidazo[1,5-a]pyridin-3-ylidene]chloropalladium(II);
cinnamyl[5-(diethylamino)-2-(2,6-diisopropylphenyl)imidazo[1,5-a]pyridin-3-ylidene]chloropalladium(II);
cinnamyl[5-(diethylamino)-2-mesitylimidazo[1,5-a]pyridin-3-ylidene]chloropalladium(II);
cinnamyl[2-(2,6-dibenzhydryl-4-methylphenyl)-5-(diethylamino)imidazo[1,5-a]pyridin-3-ylidene]chloropalladium(II);
cinnamyl[2-(2,6-diisopropylphenyl)-5-(piperidin-1-yl)imidazo[1,5-a]pyridin-3-ylidene]chloropalladium(II);
cinnamyl[2-mesityl-5-(piperidin-1-yl)imidazo[1,5-a]pyridin-3-ylidene]chloropalladium(II);
cinnamyl[2-(2,6-dibenzhydryl-4-methylphenyl)-5-(piperidin-1-yl)imidazo[1,5-a]pyridin-3-ylidene]chloropalladium(II);
cinnamyl[2-(2,6-diisopropylphenyl)-5-morpholinoimidazo[1,5-a]pyridin-3-ylidene]chloropalladium(II);
2-(2,6-diisopropylphenyl)-5-(dimethylamino)imidazo[1,5-a]pyridin-3-ylidene silver(I) chloride;
2-(2,6-diisopropylphenyl)-5-(dimethylamino)imidazo[1,5-a]pyridin-3-ylidene copper(I) chloride;
2-(2,6-diisopropylphenyl)-5-(dimethylamino)imidazo[1,5-a]pyridin-3-ylidene(cyclooctadiene)rhodium(I) chloride;
2-(2,6-diisopropylphenyl)-5-(dimethylamino)imidazo[1,5-a]pyridin-3-ylidene(cyclooctadiene)rhodium(I) triflate;
2-(2,6-diisopropylphenyl)-5-(dimethylamino)imidazo[1,5-a]pyridin-3-ylidene(bis(carbonyl))rhodium(I) triflate;
2-(2,6-diisopropylphenyl)-5-(dimethylamino)imidazo[1,5-a]pyridin-3-ylidene gold(I) bis(trifluoromethanesulfonyl)amide;
2-(2,6-diisopropylphenyl)-5-(morpholin-4-yl)imidazo[1,5-a]pyridin-3-ylidene gold(I) chloride;
2-(2,4,6-trimethylphenyl)-5-(morpholin-4-yl)imidazo[1,5-a]pyridin-3-ylidene gold(I) chloride;
2-(2,6-Diisopropylphenyl)-9-(dimethylamino)imidazo[1,5-a]quinolin-3-ylidene silver(I) chloride;
2-(2,6-Diisopropylphenyl)-9-(diethylamino)imidazo[1,5-a]quinolin-3-ylidene silver(I) chloride;
2-(2,6-Diisopropylphenyl)-9-(piperidin-1-yl)imidazo[1,5-a]quinolin-3-ylidene silver(I) chloride;
2-(2,6-Diisopropylphenyl)-9-(morpholin-4-yl)imidazo[1,5-a]quinolin-3-ylidene silver(I) chloride;
2-(2,4,6-trimethylphenyl)-9-(dimethylamino)imidazo[1,5-a]quinolin-3-ylidene silver(I) chloride;
2-(2,6-Diisopropylphenyl)-9-(dimethylamino)imidazo[1,5-a]quinolin-3-ylidene gold(I) chloride;
2-(2,6-Diisopropylphenyl)-9-(diethylamino)imidazo[1,5-a]quinolin-3-ylidene gold(I) chloride;
2-(2,6-Diisopropylphenyl)-9-(piperidin-1-yl)imidazo[1,5-a]quinolin-3-ylidene gold(I) chloride;
2-(2,6-Diisopropylphenyl)-9-(morpholin-4-yl)imidazo[1,5-a]quinolin-3-ylidene gold(I) chloride;
2-(2,4,6-trimethylphenyl)-9-(dimethylamino)imidazo[1,5-a]quinolin-3-ylidene gold(I) chloride; and
2-(2,6-Diisopropylphenyl)-9-(2,4,6-trimethylphenyl)imidazo[1,5-a]quinolin-3-ylidene gold(I) chloride.

41. A method of promoting a reaction selected from the group consisting of:
(a) a reaction between a first reagent and an aryl iodide, optionally in the presence of a Lewis acid;
(b) a reaction between an aniline and an aryl iodide, optionally in the presence of a Lewis acid;
(c) a hydroamination reaction between an alkyne and an amine, optionally in the presence of a Lewis acid;
(d) hydration of an alkyne in the presence of water;
(e) a reaction between an aryl chloride and a lithium amide;
(f) a reaction between an aryl chloride and an arylmagnesium halide;
(g) a reaction between an aroyl chloride and an aryl boronic acid in the presence of a base; and
(h) a reaction between an aryl iodide, an aniline, and carbon monoxide (CO) in the presence of a base;
wherein:
the first reagent, the aryl iodide, and the optional Lewis acid of (a);
the aniline, the aryl iodide, and the optional Lewis acid of (b);
the alkyne, the amine, and the optional Lewis acid of (c);
the alkyne, the water, and the optional Lewis acid of (d); the aryl chloride and the lithium amide of (e);
the aryl chloride and the arylmagnesium halide of (f);
the aroyl chloride and the aryl boronic acid of (g); or
the aryl iodide, the aniline, the CO, and the base of (h) are contacted in the presence of the compound of claim 21.

42-122. (canceled)

123. A method of preparing 2-(2,6-diisopropylphenyl)-5-(dimethylamino)imidazo[1,5-a]pyridin-2-ium chloride) (1): 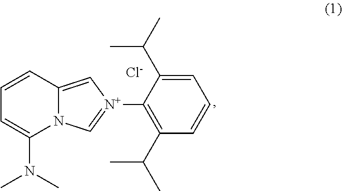 [SMILES: CC(C)c1cccc(C(C)C)c1-[n+]1cc2cccc(N(C)C)n2c1 ; [Cl-]]
the method comprising reacting (E)-6-(((2,6-diisopropylphenyl)imino)methyl)-N,N-dimethylpyridin-2-amine (Z): 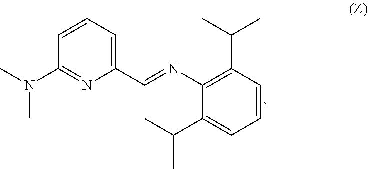 [SMILES: CC(C)c1cccc(C(C)C)c1/N=C/c1cccc(N(C)C)n1]
and paraformaldehyde so as to generate a first reaction system comprising (1).

124-145. (canceled)
